(19) United States
(12) Patent Application Publication
(10) Pub. No.: US 2019/0345522 A1
(43) Pub. Date: Nov. 14, 2019

(54) Improved Processes For Production Of Ethanol From Xylose-Containing Cellulosic Substrates Using Engineered Yeast Strains

(71) Applicant: Novozymes A/S, Bagsvaerd (DK)

(72) Inventors: Jean-Marie Mouillon, SÖDRA SANDBY (SE); Nicholas Jochumsen, Bagsvaerd (DK); José Arnau, Hellerup (DK); Leonardo De Maria, Bagsvaerd (DK); Monica Tassone, Davis, CA (US)

(73) Assignee: Novozymes A/S, Bagsvaerd (DK)

(21) Appl. No.: 16/466,426

(22) PCT Filed: Dec. 6, 2017
(86) PCT No.: PCT/US17/64885

§ 371 Date: Jun. 4, 2019

Related U.S. Application Data
(60) Provisional application No. 62/430,690, filed on Dec. 6, 2016.

(51) Int. Cl. C12P 7/10; C12N 9/92

(52) U.S. Cl. CPC C12P 7/10; C12N 9/92

(57) ABSTRACT

Provided herein are recombinant host cells having a heterologous polynucleotide encoding a hexose transporter and a heterologous polynucleotide encoding a xylose isomerase, wherein the cells are capable of fermenting xylose in the presence of glucose and under oxygen limited growth. Also described are processes of fermenting saccharified material using the recombinant cells to produce ethanol at higher yields.

### REFERENCE TO A SEQUENCE LISTING

This application contains a Sequence Listing in computer readable form, which is incorporated herein by reference.

### BACKGROUND

Ethanol is a transportation fuel commonly blending into gasoline. Cellulosic material is used as a feedstock in ethanol production processes. There are several processes in the art for making cellulose and hemicelluloses hydrolysates containing glucose, mannose, xylose and arabinose. Glucose and mannose are efficiently converted to ethanol during natural anaerobic metabolism. However, to obtain an economically relevant process at industrial scale, xylose within the hydrolysates must be fermented into ethanol.

Efforts to establish and improve pentose (e.g., xylose) utilization of the yeast Saccharomyces cerevisiae have been reported (Kim et al., 2013, Biotechnol Adv. 31(6):851-61). These include heterologous expression of xylose reductase (XR) and xylitol dehydrogenase (XDH) from naturally xylose fermenting yeasts such as Scheffersomyces (Pichia) stipitis and various Candida species, as well as the overexpression of xylulokinase (XK) and the four enzymes in the non-oxidative pentose phosphate pathway (PPP), namely transketolase (TKL), transaldolase (TAL), ribose-5-phosphate ketol-isomerase (RKI) and D-ribulose-5-phosphate 3-epimerase (RPE). Modifying the co-factor preference of S. stipitis XR towards NADH in such systems has been found to provide metabolic advantages as well as improving anaerobic growth. Pathways replacing the XR/XDH with heterologous xylose isomerase (XI) have also been reported. These and other modifications have been described in, e.g., WO2003/062430, WO2009/017441, WO2010/059095, WO2012/113120, and WO2012/135110.

Despite improvement of ethanol production processes from cellulosic material over the past decade, uptake of pentoses (e.g., xylose) across the yeast membrane remains a challenge. In one approach, S. cerevisiae host cells having a xylose reductase (XR)/xylitol dehydrogenase (XDH) pathway were engineered to overexpress various hexose transporters (HXT1, HXT2, HXT5, and HXT7) but showed poor xylose consumption (<60%) during co-fermentation of glucose and xylose (Goncalves et. al, 2014, Enzyme Microb. Technol., 63: 13-20). This study reported that the strain overexpressing HXT2 yielded incomplete anaerobic fermentations with ethanol rates significantly lower compared to strains expressing the any of other transporters (HXT1, HXT5 or HXT7). Thus, there is still a need for new industrial processes that can be used for improved ethanol production using cellulosic plant waste substrates that contain xylose, such as fermentation processes that simultaneously utilize pentoses (e.g., xylose) and glucose under oxygen limiting conditions.

### SUMMARY

Described herein are recombinant host cells comprising a heterologous polynucleotide encoding a hexose transporter (e.g., HXT2), wherein the cells are capable of fermenting pentoses (e.g., xylose). In one aspect, the recombinant cells further comprise a heterologous polynucleotide encoding a xylose isomerase.

In some embodiments, the hexose transporter has at least 60%, e.g., at least 65%, 70%, 75%, 80%, 85%, 90%, 95%, 97%, 98%, 99%, or 100% sequence identity to SEQ ID NO: 2. In some embodiments, the hexose transporter has amino acid sequence comprising or consisting of the amino acid sequence of SEQ ID NO: 2.

In some embodiments, the xylose isomerase has at least 60%, e.g., at least 65%, 70%, 75%, 80%, 85%, 90%, 95%, 97%, 98%, 99%, or 100% sequence identity to SEQ ID NO: 18. In some embodiments, the xylose isomerase has amino acid sequence comprising or consisting of the amino acid sequence of SEQ ID NO: 18.

In some embodiments, the recombinant cells are capable of higher anaerobic growth rate on a pentose (e.g. xylose) compared to an identical cell without the heterologous polynucleotide encoding a hexose transporter at about or after 4 days of incubation (e.g., under conditions described in Example 2).

In some embodiments, the recombinant cells are capable of higher pentose (e.g., xylose) consumption compared to an identical cell without the heterologous polynucleotide encoding a hexose transporter at about or after 40 hours fermentation (e.g., under conditions described in Example 3). In some embodiments, the recombinant cells are capable of consuming more than 65%, e.g., at least 70%, 75%, 80%, 85%, 90%, 95% of pentose (e.g., xylose) in the medium, and/or capable of consuming more than 65%, e.g., at least 70%, 75%, 80%, 85%, 90%, 95% of glucose in the medium, at about or after 66 hours fermentation (e.g., under conditions described in Example 4).

In some embodiments, the recombinant cells are capable of higher ethanol production compared to an identical cell without the heterologous polynucleotide encoding a hexose transporter at about or after 40 hours fermentation (e.g., under conditions described in Example 3).

In some embodiments, the recombinant cells further comprise a heterologous polynucleotide encoding a xylulokinase (XK), e.g., an XK having at least 60%, e.g., at least 65%, 70%, 75%, 80%, 85%, 90%, 95%, 97%, 98%, 99%, or 100% sequence identity to SEQ ID NO: 22.

In some embodiments, the recombinant cells further comprise a heterologous polynucleotide encoding a polypeptide selected from a ribulose 5 phosphate 3-epimerase (RPE1), a ribulose 5 phosphate isomerase (RKI1), a transketolase (TKL1), a transaldolase (TAL1).

In some embodiments, the recombinant cells comprise a disruption to one or more endogenous genes encoding a GPD and/or GPP.

In some embodiments, the recombinant cells are selected from Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cells. In some embodiment, the recombinant cells are Saccharomyces cerevisiae cells, such as derivatives of strain Saccharomyces cerevisiae CIBTS1260 (deposited under Accession No. NRRL Y-50973 at the Agricultural Research Service Culture Collection (NRRL), Illinois 61604 U.S.A.).

Also described are methods of producing ethanol using the recombinant cells. One aspect is a method for producing ethanol, comprising cultivating a recombinant cell described herein in a fermentable medium under suitable conditions to produce ethanol. In another aspect is a method for producing ethanol, comprising: (a) saccharifying a cellulosic and/or starch-containing material with an enzyme composition; and (b) fermenting the saccharified material of step (a) with a recombinant cell described herein.

In some embodiments of the methods, fermentation consumes an increased amount of glucose and pentose (e.g., xylose) when compared to fermentation using an identical cell without the heterologous polynucleotide encoding a hexose transporter under the same conditions (e.g., at about or after 40 hours fermentation, such as the conditions described in Example 3). In one embodiment, more than 65%, e.g., at least 70%, 75%, 80%, 85%, 90%, 95% of pentose (e.g. xylose) in the medium is consumed, and/or more than 65%, e.g., at least 70%, 75%, 80%, 85%, 90%, 95% of glucose in the medium is consumed, at about or after 66 hours fermentation (e.g., under conditions described in Example 4).

In some embodiments of the methods, fermentation provides higher ethanol yield when compared to fermentation using an identical cell without the heterologous polynucleotide encoding a hexose transporter under the same conditions (e.g., at about or after 40 hours fermentation, such as the conditions described in Example 3).

In some embodiments of the methods, fermentation is conducted under anaerobic conditions.

In some embodiments of the methods, the further comprises recovering the fermentation product from the fermentation.

In some embodiments of the methods, saccharification occurs on a cellulosic material, and the cellulosic material is pretreated. In some embodiments, the pretreatment is a dilute acid pretreatment.

In some embodiments of the methods, saccharification occurs on a cellulosic material, and the enzyme composition comprises one or more enzymes selected from a cellulase, an AA9 polypeptide, a hemicellulase, a CIP, an esterase, an expansin, a ligninolytic enzyme, an oxidoreductase, a pectinase, a protease, and a swollenin. In some embodiments, the cellulase is one or more enzymes selected from an endoglucanase, a cellobiohydrolase, and a beta-glucosidase. In some embodiments, the hemicellulase is one or more enzymes selected a xylanase, an acetylxylan esterase, a feruloyl esterase, an arabinofuranosidase, a xylosidase, and a glucuronidase.

In some embodiments of the methods, saccharification and fermentation are performed simultaneously in a simultaneous saccharification and fermentation (SSF). In some embodiments, saccharification and fermentation are performed sequentially (SHF).

### BRIEF DESCRIPTION OF THE FIGURES

FIG. 1 shows a plasmid map for pFYD1090.

FIG. 2 shows a plasmid map for pFYD1092.

FIG. 3 shows a plasmid map for pFYD1497.

FIG. 4 shows results from anaerobic spot tests on SD2 (glucose) and SX2 (xylose) agar plates with incubation at 30° C. A dilution series of each strain was prepared in order to spot approx. 2000, 200, 20 and 2 colony forming units (CFU) onto the plates starting with 2000 CFU on the left side of the plates.

FIG. 5 shows results from the anaerobic syringe fermentations in SX2.5 media. ♦ and ▴: xylose and EtOH concentration (g/L) for the FYD853 fermentations, respectively. ⋄ and Δ: xylose and EtOH concentration (g/L) for the FYD1547 fermentations, respectively. The solid lines indicate the median values for the FYD853 fermentations and the dotted lines indicate the median values for the FYD1547 fermentations.

FIG. 6 shows results from the anaerobic syringe fermentations in SD5X2.5 media. ▪, ♦ and ▴: glucose, xylose and EtOH concentration (g/L) for the FYD853 fermentations, respectively. □, ⋄ and Δ: glucose, xylose and EtOH concentration (g/L) for the FYD1547 fermentations, respectively. The solid lines indicate the median values for the FYD853 fermentations and the dotted lines indicate the median values for the FYD1547 fermentations.

FIG. 7 shows aerobic growth for yeast strains FYD853 and FYD1547 in SD2 (glucose) media.

FIG. 8 shows aerobic growth for yeast strains FYD853 and FYD1547 in SX1/SD1 (xylose+glucose) media.

FIG. 9 shows aerobic growth for yeast strains FYD853 and FYD1547 in SX2 (xylose) media.

FIG. 10 shows aerobic growth for yeast strains MBG4982 and McTs1084-1087 in SD2 (glucose) media.

FIG. 11 shows aerobic growth for yeast strains MBG4982 and McTs1084-1087 in SX1/SD1 (xylose+glucose) media.

FIG. 12 shows aerobic growth for yeast strains MBG4982 and McTs1084-1087 in SX2 (xylose) media.

FIG. 13 shows ethanol concentrations from fermentation samples of yeast strains FYD853 and FYD1547 in SX6, SD6, or SX3/SD3 media.

FIG. 14 shows ethanol concentrations from fermentation samples of yeast strains MBG4982 and McTs1084-1087 in SX6, SD6, or SX3/SD3 media.

FIG. 15 shows growth in SD2 media for strains having the XR/XDH xylose utilization pathway of the P51-F11 background.

FIG. 16 shows growth in SX1/SD1 media for strains having the XR/XDH xylose utilization pathway of the P51-F11 background.

FIG. 17 shows growth in SX2 media for strains having the XR/XDH xylose utilization pathway of the P51-F11 background.

FIG. 18 shows growth in SD2 media for strains having the XR/XDH xylose utilization pathway of the P52-B02 background.

FIG. 19 shows growth in SX1/SD1 media for strains having the XR/XDH xylose utilization pathway of the P52-B02 background.

FIG. 20 shows growth in SX2 media for strains having the XR/XDH xylose utilization pathway of the P52-B02 background.

FIG. 21 shows growth in SD2 media for strains having the XR/XDH xylose utilization pathway of the P55-H01 background.

FIG. 22 shows growth in SX1/SD1 media for strains having the XR/XDH xylose utilization pathway of the P55-H01 background.

FIG. 23 shows growth in SX2 media for strains having the XR/XDH xylose utilization pathway of the P55-H01 background.

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Auxiliary Activity 9: The term “Auxiliary Activity 9” or “AA9” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40 C-80° C., e.g., 50° C., 55° C., 60° C., 65° C., or 70° C., and a suitable pH, such as 4-9, e.g., 4.5, 5.0, 5.5, 6.0, 6.5, 7.0, 7.5, 8.0, or 8.5, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5 L (Novozymes A/S, Bagsværd, Denmark) and beta-glucosidase as the source of the cellulolytic activity, wherein the beta-glucosidase is present at a weight of at least 2-5% protein of the cellulase protein loading. In one embodiment, the beta-glucosidase is an Aspergillus oryzae beta-glucosidase (e.g., recombinantly produced in Aspergillus oryzae according to WO 02/095014). In another embodiment, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Catalase: The term “catalase” means a hydrogen-peroxide:hydrogen-peroxide oxidoreductase (EC 1.11.1.6) that catalyzes the conversion of 2H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme composition or cellulase: The term “cellulolytic enzyme composition” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N21 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one embodiment, the cellulosic material is any biomass material. In another embodiment, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, switchgrass, or wheat straw.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred embodiment, the cellulosic material is pretreated.

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Disruption: The term “disruption” means that a coding region and/or control sequence of a referenced gene is partially or entirely modified (such as by deletion, insertion, and/or substitution of one or more nucleotides) resulting in the absence (inactivation) or decrease in expression, and/or the absence or decrease of enzyme activity of the encoded polypeptide. The effects of disruption can be measured using techniques known in the art such as detecting the absence or decrease of enzyme activity using from cell-free extract measurements referenced herein; or by the absence or decrease of corresponding mRNA (e.g., at least 25% decrease, at least 50% decrease, at least 60% decrease, at least 70% decrease, at least 80% decrease, or at least 90% decrease); the absence or decrease in the amount of corresponding polypeptide having enzyme activity (e.g., at least 25% decrease, at least 50% decrease, at least 60% decrease, at least 70% decrease, at least 80% decrease, or at least 90% decrease); or the absence or decrease of the specific activity of the corresponding polypeptide having enzyme activity (e.g., at least 25% decrease, at least 50% decrease, at least 60% decrease, at least 70% decrease, at least 80% decrease, or at least 90% decrease). Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)).

Endogenous gene: The term “endogenous gene” means a gene that is native to the referenced host cell. “Endogenous gene expression” means expression of an endogenous gene.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences, wherein the control sequences provide for expression of the polynucleotide encoding the polypeptide. At a minimum, the expression vector comprises a promoter sequence, and transcriptional and translational stop signal sequences.

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as ethanol. In some instances, the fermentation medium is derived from a natural source, such as sugar cane, starch, or cellulose, and may be the result of pretreating the source by enzymatic hydrolysis (saccharification). The term fermentation medium is understood herein to refer to a medium before the fermenting microorganism(s) is(are) added, such as, a medium resulting from a saccharification process, as well as a medium used in a simultaneous saccharification and fermentation process (SSF).

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.−80° C., e.g., 50° C., 55° C., 60° C., 65° C., or 70° C., and a suitable pH such as 4-9, e.g., 5.0, 5.5, 6.0, 6.5, or 7.0.

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Host cell: The term “host cell” means any cell type that is susceptible to transformation, transfection, transduction, and the like with a nucleic acid construct or expression vector. The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Nucleic acid construct: The term “nucleic acid construct” means a polynucleotide comprises one or more (e.g., two, several) control sequences. The polynucleotide may be single-stranded or double-stranded, and may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Pentose: The term “pentose” means a five carbon monosaccharide (e.g., xylose, arabinose, ribose, lyxose, ribulose, and xylulose). Pentoses, such as D-xylose and L-arabinose, may be derived, e.g., through saccharification of a plant cell wall polysaccharide.

Pretreated corn stover: The term “Pretreated Corn Stover” or “PCS” means a cellulosic material derived from corn stover by treatment with heat and dilute sulfuric acid, alkaline pretreatment, neutral pretreatment, or any pretreatment known in the art.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, J. Mol. Biol. 1970, 48, 443-453) as implemented in the Needle program of the EMBOSS package (EMBOSS: The European Molecular Biology Open Software Suite, Rice et al., Trends Genet 2000, 16, 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of the Referenced Sequence−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Referenced Sequence−Total Number of Gaps in Alignment)

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylose Isomerase: The term “Xylose Isomerase” or “XI” means an enzyme which can catalyze D-xylose into D-xylulose in vivo, and convert D-glucose into D-fructose in vitro. Xylose isomerase is also known as “glucose isomerase” and is classified as E.C. 5.3.1.5. As the structure of the enzyme is very stable, the xylose isomerase is one of the good models for studying the relationships between protein structure and functions (Karimaki et al., Protein Eng Des Sel, 12004, 17 (12):861-869). Moreover, the extremely important industrial application value makes the xylose isomerase is seen as important industrial enzyme as protease and amylase (Tian Shen et al., Microbiology Bulletin, 2007, 34 (2): 355-358; Bhosale et al., Microbiol Rev, 1996, 60 (2): 280-300). The scientists keep high concern and carried out extensive research on xylose isomerase. Since 1970s, the applications of the xylose isomerase have focused on the production of high fructose syrup and fuel ethanol. In recent years, scientists have found that under certain conditions, the xylose isomerase can be used for producing many important rare sugars, which are the production materials in the pharmaceutical industry, such as ribose, mannose, arabinose and lyxose (Karlmaki et al., Protein Eng Des Se, 12004, 17 (12): 861-869). These findings bring new vitality in the research on the xylose isomerase.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the embodiments described herein include “consisting” and/or “consisting essentially of” embodiments.

Unless defined otherwise or clearly indicated by context, all technical and scientific terms used herein have the same meaning as commonly understood by one of ordinary skill in the art.

### DETAILED DESCRIPTION

Described herein, inter alia, are recombinant cells, such as yeast, capable of simultaneously converting hexoses and pentoses into ethanol, e.g., in processes as described below. The Applicant has found that suitable expression of the HXT2 hexose transporter in a cell, such as Saccharomyces cerevisiae yeast, also expressing a xylose isomerase shows surprisingly high growth on xylose, increased xylose consumption in the presence of glucose, elevated glucose consumption and improved ethanol production under anaerobic growth conditions.

In one aspect is a recombinant cell (e.g., yeast) comprising a heterologous polynucleotide encoding a hexose transporter, and wherein the yeast cell is capable of fermenting xylose.

In one embodiment, the hexose transporter comprises or consists of the amino acid sequence of the HXT2 transporter of SEQ ID NO: 2. In another embodiment, the hexose transporter is a fragment of the polypeptide of HXT2 transporter of SEQ ID NO: 2 (e.g., wherein the fragment has hexose transporter activity). In one embodiment, the number of amino acid residues in the fragment is at least 75%, e.g., at least 80%, 85%, 90%, or 95% of the number of amino acid residues in SEQ ID NO: 2.

The hexose transporter may be a variant of the HXT2 transporter of SEQ ID NO: 2. In one embodiment, the hexose transporter has at least 60%, e.g., at least 65%, 70%, 75%, 80%, 85%, 90%, 95%, 97%, 98%, 99%, or 100% sequence identity to the HXT2 transporter of SEQ ID NO: 2.

In one embodiment, the hexose transporter sequence differs by no more than ten amino acids, e.g., by no more than five amino acids, by no more than four amino acids, by no more than three amino acids, by no more than two amino acids, or by one amino acid from amino acid sequence of the HXT2 transporter of SEQ ID NO: 2. In one embodiment, the hexose transporter has an amino acid substitution, deletion, and/or insertion of one or more (e.g., two, several) of amino acid sequence of the HXT2 transporter of SEQ ID NO: 2. In some embodiments, the total number of amino acid substitutions, deletions and/or insertions is not more than 10, e.g., not more than 9, 8, 7, 6, 5, 4, 3, 2, or 1

The amino acid changes are generally of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the hexose transporter, alter the substrate specificity, change the pH optimum, and the like.

Essential amino acids can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with other hexose transporters that are related to the referenced hexose transporter.

Additional guidance on the structure-activity relationship of the hexose transporters herein can be determined using multiple sequence alignment (MSA) techniques well-known in the art. Based on the teachings herein, the skilled artisan could make similar alignments with any number of hexose transporters described herein or known in the art. Such alignments aid the skilled artisan to determine potentially relevant domains (e.g., binding domains or catalytic domains), as well as which amino acid residues are conserved and not conserved among the different hexose transporter sequences. It is appreciated in the art that changing an amino acid that is conserved at a particular position between disclosed polypeptides will more likely result in a change in biological activity (Bowie et al., 1990, Science 247: 1306-1310: “Residues that are directly involved in protein functions such as binding or catalysis will certainly be among the most conserved”). In contrast, substituting an amino acid that is not highly conserved among the polypeptides will not likely or significantly alter the biological activity.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active hexose transporters can be recovered from the host cells and rapidly sequenced using standard methods in the art. These methods allow the rapid determination of the importance of individual amino acid residues in a polypeptide.

In another embodiment, the heterologous polynucleotide encoding the hexose transporter comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to nucleotides of SEQ ID NO: 1.

In one embodiment, the heterologous polynucleotide encoding the hexose transporter comprises or consists of the coding sequence of SEQ ID NO: 1. In another embodiment, the heterologous polynucleotide encoding the hexose transporter comprises a subsequence of the coding sequence of SEQ ID NO: 1 (e.g., wherein the subsequence encodes a polypeptide having hexose transporter activity). In another embodiment, the number of nucleotides residues in the coding subsequence is at least 75%, e.g., at least 80%, 85%, 90%, or 95% of the number of the referenced coding sequence.

The referenced coding sequence of any related aspect or embodiment described herein can be the native coding sequence or a degenerate sequence, such as a codon-optimized coding sequence designed for a particular host cell.

The polynucleotide coding sequence of SEQ ID NO: 1, or a subsequence thereof, as well as the polypeptide of SEQ ID NO: 2, or a fragment thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1, or a subsequence thereof, the carrier material is used in a Southern blot.

In one embodiment, the nucleic acid probe is a polynucleotide comprising SEQ ID NO: 1; or a subsequence thereof. In another embodiment, the nucleic acid probe is a polynucleotide that encodes the polypeptide of SEQ ID NO: 2; or a fragment thereof.

For purposes of the probes described above, hybridization indicates that the polynucleotide hybridizes to a labeled nucleic acid probe, or the full-length complementary strand thereof, or a subsequence of the foregoing; under very low to very high stringency conditions. Molecules to which the nucleic acid probe hybridizes under these conditions can be detected using, for example, X-ray film. Stringency and washing conditions are defined as described supra.

In one embodiment, the hexose transporter is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 1. (Sambrook et al., 1989, Molecular Cloning, A Laboratory Manual, 2d edition, Cold Spring Harbor, N.Y.).

The hexose transporter may be obtained from microorganisms of any suitable genus, including those readily available within the UniProtKB database (www.uniprot.org).

The hexose transporter may be a bacterial hexose transporter. For example, the hexose transporter may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces hexose transporter, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma hexose transporter.

In one embodiment, the hexose transporter is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis hexose transporter.

In another embodiment, the hexose transporter is a Streptococcus equisimilis, Streptococcus pyogenes, Streptococcus uberis, or Streptococcus equi subsp. Zooepidemicus hexose transporter.

In another embodiment, the hexose transporter is a Streptomyces achromogenes, Streptomyces avermitilis, Streptomyces coelicolor, Streptomyces griseus, or Streptomyces lividans hexose transporter.

The hexose transporter may be a fungal hexose transporter. For example, the hexose transporter may be a yeast hexose transporter such as a Candida, Kluyveromyces, Pichia, Saccharomyces, Schizosaccharomyces, Yarrowia or Issatchenkia hexose transporter; or a filamentous fungal hexose transporter such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria hexose transporter.

In another embodiment, the hexose transporter is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis hexose transporter.

In another embodiment, the hexose transporter is from Saccharomyces, such as the Saccharomyces cerevisiae hexose transporter of SEQ ID NO: 2.

In another embodiment, the hexose transporter is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride hexose transporter.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

The hexose transporters may also be identified and obtained from other sources including microorganisms isolated from nature (e.g., soil, composts, water, silage, etc.) or DNA samples obtained directly from natural materials (e.g., soil, composts, water, silage, etc.) using the above-mentioned probes. Techniques for isolating microorganisms and DNA directly from natural habitats are well known in the art. The polynucleotide encoding a hexose transporter may then be derived by similarly screening a genomic or cDNA library of another microorganism or mixed DNA sample.

Once a polynucleotide encoding a hexose transporter has been detected with a suitable probe as described herein, the sequence may be isolated or cloned by utilizing techniques that are known to those of ordinary skill in the art (see, e.g., Sambrook et al., 1989, supra). Techniques used to isolate or clone polynucleotides encoding hexose transporters include isolation from genomic DNA, preparation from cDNA, or a combination thereof. The cloning of the polynucleotides from such genomic DNA can be effected, e.g., by using the well-known polymerase chain reaction (PCR) or antibody screening of expression libraries to detect cloned DNA fragments with shares structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used.

The hexose transporter may be a fused polypeptide or cleavable fusion polypeptide in which another polypeptide is fused at the N-terminus or the C-terminus of the hexose transporter. A fused polypeptide may be produced by fusing a polynucleotide encoding another polypeptide to a polynucleotide encoding the hexose transporter. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

In one aspect, the recombinant cell (e.g., yeast cell) further comprises a heterologous polynucleotide encoding a xylose isomerase (XI). The xylose isomerase may be any xylose isomerase that is suitable for the host cells and the methods described herein, such as a naturally occurring xylose isomerase or a variant thereof that retains xylose isomerase activity. In one embodiment, the xylose isomerase is present in the cytosol of the host cells.

In some embodiments, the recombinant cells comprising a heterologous polynucleotide encoding a xylose isomerase have an increased level of xylose isomerase activity compared to the host cells without the heterologous polynucleotide encoding the xylose isomerase, when cultivated under the same conditions. In some embodiments, the host cells have an increased level of xylose isomerase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the xylose isomerase, when cultivated under the same conditions.

Exemplary xylose isomerases that can be used with the recombinant host cells and methods of use described herein include, but are not limited to, Xls from the fungus Piromyces sp. (WO2003/062430) or other sources (Madhavan et al., 2009, Appl Microbiol Biotechnol. 82(6), 1067-1078) have been expressed in S. cerevisiae host cells. Still other Xls suitable for expression in yeast have been described in US 2012/0184020 (an XI from Ruminococcus flavefaciens), WO2011/078262 (several Xls from Reticulitermes speratus and Mastotermes darwiniensis) and WO2012/009272 (constructs and fungal cells containing an XI from Abiotrophia defectiva). U.S. Pat. No. 8,586,336 describes a S. cerevisiae host cell expressing an XI obtained by bovine rumen fluid (shown herein as SEQ ID NO: 18).

Additional polynucleotides encoding suitable xylose isomerases may be obtained from microorganisms of any genus, including those readily available within the UniProtKB database (www.uniprot.org). In one embodiment, the xylose isomerases is a bacterial, a yeast, or a filamentous fungal xylose isomerase, e.g., obtained from any of the microorganisms described herein, as described supra under the sections related to hexose transporters.

The xylose isomerase coding sequences can also be used to design nucleic acid probes to identify and clone DNA encoding xylose isomerases from strains of different genera or species, as described supra.

The polynucleotides encoding xylose isomerases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding xylose isomerases are described supra.

In one embodiment, the xylose isomerase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any xylose isomerase described herein (e.g., the xylose isomerase of SEQ ID NO: 18). In one aspect, the xylose isomerase sequence differs by no more than ten amino acids, e.g., by no more than five amino acids, by no more than four amino acids, by no more than three amino acids, by no more than two amino acids, or by one amino acid from any xylose isomerase described herein (e.g., the xylose isomerase of SEQ ID NO: 18). In one embodiment, the xylose isomerase comprises or consists of the amino acid sequence of any xylose isomerase described herein (e.g., the xylose isomerase of SEQ ID NO: 18), allelic variant, or a fragment thereof having xylose isomerase activity. In one embodiment, the xylose isomerase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the xylose isomerase has at least 20%, e.g., at least 40%, at least 50%, at least 60%, at least 70%, at least 80%, at least 90%, at least 95%, at least 96%, at least 97%, at least 98%, at least 99%, or 100% of the xylose isomerase activity of any xylose isomerase described herein (e.g., the xylose isomerase of SEQ ID NO: 18) under the same conditions.

In one embodiment, the xylose isomerase coding sequence hybridizes under at least low stringency conditions, e.g., medium stringency conditions, medium-high stringency conditions, high stringency conditions, or very high stringency conditions with the full-length complementary strand of the coding sequence from any xylose isomerase described herein (e.g., the xylose isomerase of SEQ ID NO: 18). In one embodiment, the xylose isomerase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any xylose isomerase described herein (e.g., the xylose isomerase of SEQ ID NO: 18).

In one embodiment, the heterologous polynucleotide encoding the xylose isomerase comprises the coding sequence of any xylose isomerase described herein (e.g., the xylose isomerase of SEQ ID NO: 18). In one embodiment, the heterologous polynucleotide encoding the xylose isomerase comprises a subsequence of the coding sequence from any xylose isomerase described herein, wherein the subsequence encodes a polypeptide having xylose isomerase activity. In one embodiment, the number of nucleotides residues in the subsequence is at least 75%, e.g., at least 80%, 85%, 90%, or 95% of the number of the referenced coding sequence.

The xylose isomerases can also include fused polypeptides or cleavable fusion polypeptides, as described supra.

In one aspect, the recombinant cell (e.g., yeast cell) further comprises a heterologous polynucleotide encoding a xylulokinase (XK). A xylulokinase, as used herein, provides enzymatic activity for converting D-xylulose to xylulose 5-phosphate. The xylulokinase may be any xylulokinase that is suitable for the host cells and the methods described herein, such as a naturally occurring xylulokinase or a variant thereof that retains xylulokinase activity. In one embodiment, the xylulokinase is present in the cytosol of the host cells.

In some embodiments, the recombinant cells comprising a heterologous polynucleotide encoding a xylulokinase have an increased level of xylulokinase activity compared to the host cells without the heterologous polynucleotide encoding the xylulokinase, when cultivated under the same conditions. In some embodiments, the host cells have an increased level of xylose isomerase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the xylulokinase, when cultivated under the same conditions.

Exemplary xylulokinases that can be used with the recombinant host cells and methods of use described herein include, but are not limited to, the Saccharomyces cerevisiae xylulokinase of SEQ ID NO: 22. Additional polynucleotides encoding suitable xylulokinases may be obtained from microorganisms of any genus, including those readily available within the UniProtKB database (www.uniprot.org). In one embodiment, the xylulokinases is a bacterial, a yeast, or a filamentous fungal xylulokinase, e.g., obtained from any of the microorganisms described herein, as described supra under the sections related to hexose transporters.

The xylulokinase coding sequences can also be used to design nucleic acid probes to identify and clone DNA encoding xylulokinases from strains of different genera or species, as described supra.

The polynucleotides encoding xylulokinases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding xylulokinases are described supra.

In one embodiment, the xylulokinase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any xylulokinase described herein (e.g., the Saccharomyces cerevisiae xylulokinase of SEQ ID NO: 22). In one embodiment, the xylulokinase sequence differs by no more than ten amino acids, e.g., by no more than five amino acids, by no more than four amino acids, by no more than three amino acids, by no more than two amino acids, or by one amino acid from any xylulokinase described herein (e.g., the Saccharomyces cerevisiae xylulokinase of SEQ ID NO: 22). In one embodiment, the xylulokinase comprises or consists of the amino acid sequence of any xylulokinase described herein (e.g., the Saccharomyces cerevisiae xylulokinase of SEQ ID NO: 22), allelic variant, or a fragment thereof having xylulokinase activity. In one embodiment, the xylulokinase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the xylulokinase has at least 20%, e.g., at least 40%, at least 50%, at least 60%, at least 70%, at least 80%, at least 90%, at least 95%, at least 96%, at least 97%, at least 98%, at least 99%, or 100% of the xylulokinase activity of any xylulokinase described herein (e.g., the Saccharomyces cerevisiae xylulokinase of SEQ ID NO: 22) under the same conditions.

In one embodiment, the xylulokinase coding sequence hybridizes under at least low stringency conditions, e.g., medium stringency conditions, medium-high stringency conditions, high stringency conditions, or very high stringency conditions with the full-length complementary strand of the coding sequence from any xylulokinase described herein (e.g., the Saccharomyces cerevisiae xylulokinase of SEQ ID NO: 22). In one embodiment, the xylulokinase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any xylulokinase described herein (e.g., the Saccharomyces cerevisiae xylulokinase of SEQ ID NO: 22).

In one embodiment, the heterologous polynucleotide encoding the xylulokinase comprises the coding sequence of any xylulokinase described herein (e.g., the Saccharomyces cerevisiae xylulokinase of SEQ ID NO: 22). In one embodiment, the heterologous polynucleotide encoding the xylulokinase comprises a subsequence of the coding sequence from any xylulokinase described herein, wherein the subsequence encodes a polypeptide having xylulokinase activity. In one embodiment, the number of nucleotides residues in the subsequence is at least 75%, e.g., at least 80%, 85%, 90%, or 95% of the number of the referenced coding sequence.

The xylulokinases can also include fused polypeptides or cleavable fusion polypeptides, as described supra.

In one aspect, the recombinant cell (e.g., yeast cell) further comprises a heterologous polynucleotide encoding a ribulose 5 phosphate 3-epimerase (RPE1). A ribulose 5 phosphate 3-epimerase, as used herein, provides enzymatic activity for converting L-ribulose 5-phosphate to L-xylulose 5-phosphate (EC 5.1.3.22). The RPE1 may be any RPE1 that is suitable for the host cells and the methods described herein, such as a naturally occurring RPE1 or a variant thereof that retains RPE1 activity. In one embodiment, the RPE1 is present in the cytosol of the host cells.

In one embodiment, the recombinant cell comprises a heterologous polynucleotide encoding a ribulose 5 phosphate 3-epimerase (RPE1), wherein the RPE1 is Saccharomyces cerevisiae RPE1, or an RPE1 having at least 60%, e.g., at least 65%, 70%, 75%, 80%, 85%, 90%, 95%, 97%, 98%, 99%, or 100% sequence identity to a Saccharomyces cerevisiae RPE1.

In one aspect, the recombinant cell (e.g., yeast cell) further comprises a heterologous polynucleotide encoding a ribulose 5 phosphate isomerase (RKI1). A ribulose 5 phosphate isomerase, as used herein, provides enzymatic activity for converting ribose-5-phosphate to ribulose 5-phosphate. The RKI1 may be any RKI1 that is suitable for the host cells and the methods described herein, such as a naturally occurring RKI1 or a variant thereof that retains RKI1 activity. In one embodiment, the RKI1 is present in the cytosol of the host cells.

In one embodiment, the recombinant cell comprises a heterologous polynucleotide encoding a ribulose 5 phosphate isomerase (RKI1), wherein the RKI1 is a Saccharomyces cerevisiae RKI1, or an RKI1 having at least 60%, e.g., at least 65%, 70%, 75%, 80%, 85%, 90%, 95%, 97%, 98%, 99%, or 100% sequence identity to a Saccharomyces cerevisiae RKI1.

In one aspect, the recombinant cell (e.g., yeast cell) further comprises a heterologous polynucleotide encoding a transketolase (TKL1). The TKL1 may be any TKL1 that is suitable for the host cells and the methods described herein, such as a naturally occurring TKL1 or a variant thereof that retains TKL1 activity. In one embodiment, the TKL1 is present in the cytosol of the host cells.

In one embodiment, the recombinant cell comprises a heterologous polynucleotide encoding a transketolase (TKL1), wherein the TKL1 is a Saccharomyces cerevisiae TKL1, or a TKL1 having at least 60%, e.g., at least 65%, 70%, 75%, 80%, 85%, 90%, 95%, 97%, 98%, 99%, or 100% sequence identity to a Saccharomyces cerevisiae TKL1.

In one aspect, the recombinant cell (e.g., yeast cell) further comprises a heterologous polynucleotide encoding a transaldolase (TAL1). The TAL1 may be any TAL1 that is suitable for the host cells and the methods described herein, such as a naturally occurring TAL1 or a variant thereof that retains TAL1 activity. In one embodiment, the TAL1 is present in the cytosol of the host cells.

In one embodiment, the recombinant cell comprises a heterologous polynucleotide encoding a transketolase (TAL1), wherein the TAL1 is a Saccharomyces cerevisiae TAL1, or a TAL1 having at least 60%, e.g., at least 65%, 70%, 75%, 80%, 85%, 90%, 95%, 97%, 98%, 99%, or 100% sequence identity to a Saccharomyces cerevisiae TAL1.

In one aspect, the recombinant cells described herein (e.g., a cell comprising a heterologous polynucleotide encoding a hexose transporter described herein and xylose isomerase) have improved anaerobic growth on a pentose (e.g., xylose). In one embodiment, the recombinant cell is capable of higher anaerobic growth rate on a pentose (e.g., xylose) compared to the same cell without the heterologous polynucleotide encoding a hexose transporter at about or after 4 days of incubation (e.g., under conditions described in Example 2).

In one aspect, the recombinant cells described herein (e.g., a cell comprising a heterologous polynucleotide encoding a hexose transporter described herein and xylose isomerase) have higher pentose (e.g., xylose) consumption. In one embodiment, the recombinant cell is capable of higher pentose (e.g., xylose) consumption compared to the same cell without the heterologous polynucleotide encoding a hexose transporter at about or after 40 hours fermentation (e.g., under conditions described in Example 3). In one embodiment, the recombinant cell is capable of consuming more than 65%, e.g., at least 70%, 75%, 80%, 85%, 90%, 95% of pentose (e.g., xylose) in the medium at about or after 66 hours fermentation (e.g., under conditions described in Example 4). In one embodiment, the recombinant cell is capable of consuming more than 65%, e.g., at least 70%, 75%, 80%, 85%, 90%, 95% of glucose in the medium at about or after 66 hours fermentation (e.g., under conditions described in Example 4). In one embodiment, the recombinant cell is capable of consuming more than 65%, e.g., at least 70%, 75%, 80%, 85%, 90%, 95% of pentose (e.g., xylose) in the medium, and is capable of consuming more than 65%, e.g., at least 70%, 75%, 80%, 85%, 90%, 95% of glucose in the medium, at about or after 66 hours fermentation (e.g., under conditions described in Example 4).

In one aspect, the recombinant cells described herein (e.g., a cell comprising a heterologous polynucleotide encoding a hexose transporter described herein and xylose isomerase) have higher ethanol production. In one embodiment, the recombinant cell is capable of higher ethanol production compared to the same cell without the heterologous polynucleotide encoding a hexose transporter at about or after 40 hours fermentation (e.g., under conditions described in Example 3).

### Gene Disruptions

The recombinant cells described herein may also comprise one or more (e.g., two, several) gene disruptions, e.g., to divert sugar metabolism from undesired products to ethanol. In some aspects, the recombinant host cells produce a greater amount of ethanol compared to the cell without the one or more disruptions when cultivated under identical conditions. In some aspects, one or more of the disrupted endogenous genes is inactivated.

In certain embodiments, the recombinant cells provided herein comprise a disruption of one or more endogenous genes encoding enzymes involved in producing alternate fermentative products such as glycerol or other byproducts such as acetate or diols. For example, the cells provided herein may comprise a disruption of one or more of glycerol 3-phosphate dehydrogenase (GPD, catalyzes reaction of dihydroxyacetone phosphate to glycerol 3-phosphate), glycerol 3-phosphatase (GPP, catalyzes conversion of glycerol-3 phosphate to glycerol), glycerol kinase (catalyzes conversion of glycerol 3-phosphate to glycerol), dihydroxyacetone kinase (catalyzes conversion of dihydroxyacetone phosphate to dihydroxyacetone), glycerol dehydrogenase (catalyzes conversion of dihydroxyacetone to glycerol), and aldehyde dehydrogenase (ALD, e.g., converts acetaldehyde to acetate).

Modeling analysis can be used to design gene disruptions that additionally optimize utilization of the pathway. One exemplary computational method for identifying and designing metabolic alterations favoring biosynthesis of a desired product is the OptKnock computational framework, Burgard et al., 2003, Biotechnol. Bioeng. 84: 647-657.

The recombinant cells comprising a gene disruption may be constructed using methods well known in the art, including those methods described herein. A portion of the gene can be disrupted such as the coding region or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, but are not limited to, a leader, propeptide sequence, signal sequence, transcription terminator, and transcriptional activator.

The recombinant cells comprising a gene disruption may be constructed by gene deletion techniques to eliminate or reduce expression of the gene. Gene deletion techniques enable the partial or complete removal of the gene thereby eliminating their expression. In such methods, deletion of the gene is accomplished by homologous recombination using a plasmid that has been constructed to contiguously contain the 5′ and 3′ regions flanking the gene.

The recombinant cells comprising a gene disruption may also be constructed by introducing, substituting, and/or removing one or more (e.g., two, several) nucleotides in the gene or a control sequence thereof required for the transcription or translation thereof. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortle, 1985, Science 229: 4719; Lo et al., 1985, Proc. Natl. Acad. Sci. U.S.A. 81: 2285; Higuchi et al., 1988, Nucleic Acids Res 16: 7351; Shimada, 1996, Meth. Mol. Biol. 57: 157; Ho et al., 1989, Gene 77: 61; Horton et al., 1989, Gene 77: 61; and Sarkar and Sommer, 1990, BioTechniques 8: 404.

The recombinant cells comprising a gene disruption may also be constructed by inserting into the gene a disruptive nucleic acid construct comprising a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The recombinant cells comprising a gene disruption may also be constructed by the process of gene conversion (see, for example, Iglesias and Trautner, 1983, Molecular General Genetics 189: 73-76). For example, in the gene conversion method, a nucleotide sequence corresponding to the gene is mutagenized in vitro to produce a defective nucleotide sequence, which is then transformed into the recombinant strain to produce a defective gene. By homologous recombination, the defective nucleotide sequence replaces the endogenous gene. It may be desirable that the defective nucleotide sequence also comprises a marker for selection of transformants containing the defective gene.

The recombinant cells comprising a gene disruption may be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of the gene may be performed by subjecting the parent strain to mutagenesis and screening for mutant strains in which expression of the gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the parent strain to be mutagenized in the presence of the mutagenizing agent of choice under suitable conditions, and selecting for mutants exhibiting reduced or no expression of the gene.

A nucleotide sequence homologous or complementary to a gene described herein may be used from other microbial sources to disrupt the corresponding gene in a recombinant strain of choice.

In one aspect, the modification of a gene in the recombinant cell is unmarked with a selectable marker. Removal of the selectable marker gene may be accomplished by culturing the mutants on a counter-selection medium. Where the selectable marker gene contains repeats flanking its 5′ and 3′ ends, the repeats will facilitate the looping out of the selectable marker gene by homologous recombination when the mutant strain is submitted to counter-selection. The selectable marker gene may also be removed by homologous recombination by introducing into the mutant strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

### Hosts Cells and Recombinant Methods

The recombinant cells described herein may be selected from any host cell capable of ethanol fermentation. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The host cells for preparing the recombinant cells described herein can be from any suitable host, such as a yeast strain, including, but not limited to, a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cell. In particular, Saccharomyces host cells are contemplated, such as Saccharomyces cerevisiae, bayanus or carlsbergensis cells. Preferably, the yeast cell is a Saccharomyces cerevisiae cell. Suitable cells can, for example, be derived from commercially available strains and polyploid or aneuploid industrial strains, including but not limited to those from Superstart™, THERMOSACC®, C5 FUEL™, XyloFerm®, etc. (Lallemand); RED STAR and ETHANOL RED® (Fermentis/Lesaffre); FALI (AB Mauri); Baker's Best Yeast, Baker's Compressed Yeast, etc. (Fleishmann's Yeast); BIOFERM AFT, XP, CF, and XR (North American Bioproducts Corp.); Turbo Yeast (Gert Strand AB); and FERMIOL® (DSM Specialties). Other useful yeast strains are available from biological depositories such as the American Type Culture Collection (ATCC) or the Deutsche Sammlung von Mikroorganismen and Zellkulturen GmbH (DSMZ), such as, e.g., BY4741 (e.g., ATCC 201388); Y108-1 (ATCC PTA. 10567) and NRRL YB-1952 (ARS Culture Collection). Still other S. cerevisiae strains suitable as host cells DBY746, [Alpha][Eta]22, 5150-2B, GPY55-15Ba, CEN. PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The recombinant cells described herein may utilize expression vectors comprising the coding sequence of one or more (e.g., two, several) heterologous genes linked to one or more control sequences that direct expression in a suitable cell under conditions compatible with the control sequence(s). Such expression vectors may be used in any of the cells and methods described herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

A construct or vector (or multiple constructs or vectors) comprising the one or more (e.g., two, several) heterologous genes may be introduced into a cell so that the construct or vector is maintained as a chromosomal integrant or as a self-replicating extra-chromosomal vector as described earlier.

The various nucleotide and control sequences may be joined together to produce a recombinant expression vector that may include one or more (e.g., two, several) convenient restriction sites to allow for insertion or substitution of the polynucleotide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the cell, or a transposon, may be used.

The expression vector may contain any suitable promoter sequence that is recognized by a cell for expression of a gene described herein. The promoter sequence contains transcriptional control sequences that mediate the expression of the polypeptide. The promoter may be any polynucleotide that shows transcriptional activity in the cell of choice including mutant, truncated, and hybrid promoters, and may be obtained from genes encoding extracellular or intracellular polypeptides either homologous or heterologous to the cell.

Each heterologous polynucleotide described herein may be operably linked to a promoter that is foreign to the polynucleotide. For example, in one embodiment, the heterologous polynucleotide encoding the hexose transporter is operably linked to a promoter foreign to the polynucleotide. The promoters may be identical to or share a high degree of sequence identity (e.g., at least about 80%, at least about 85%, at least about 90%, at least about 95%, or at least about 99%) with a selected native promoter.

Examples of suitable promoters for directing the transcription of the nucleic acid constructs in a yeast cells, include, but are not limited to, the promoters obtained from the genes for enolase, (e.g., S. cerevisiae enolase or I. orientalis enolase (ENO1)), galactokinase (e.g., S. cerevisiae galactokinase or I. orientalis galactokinase (GAL1)), alcohol dehydrogenase/glyceraldehyde-3-phosphate dehydrogenase (e.g., S. cerevisiae alcohol dehydrogenase/glyceraldehyde-3-phosphate dehydrogenase or I. orientalis alcohol dehydrogenase/glyceraldehyde-3-phosphate dehydrogenase (ADH1, ADH2/GAP)), triose phosphate isomerase (e.g., S. cerevisiae triose phosphate isomerase or I. orientalis triose phosphate isomerase (TPI)), metallothionein (e.g., S. cerevisiae metallothionein or I. orientalis metallothionein (CUP1)), 3-phosphoglycerate kinase (e.g., S. cerevisiae 3-phosphoglycerate kinase or I. orientalis 3-phosphoglycerate kinase (PGK)), PDC1, xylose reductase (XR), xylitol dehydrogenase (XDH), L-(+)-lactate-cytochrome c oxidoreductase (CYB2), translation elongation factor-1 (TEF1), translation elongation factor-2 (TEF2), glyceraldehyde-3-phosphate dehydrogenase (GAPDH), and orotidine 5′-phosphate decarboxylase (URA3) genes. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the yeast cell of choice may be used. The terminator may be identical to or share a high degree of sequence identity (e.g., at least about 80%, at least about 85%, at least about 90%, at least about 95%, or at least about 99%) with the selected native terminator.

Suitable terminators for yeast host cells may be obtained from the genes for enolase (e.g., S. cerevisiae or I. orientalis enolase cytochrome C (e.g., S. cerevisiae or I. orientalis cytochrome (CYC1)), glyceraldehyde-3-phosphate dehydrogenase (e.g., S. cerevisiae or I. orientalis glyceraldehyde-3-phosphate dehydrogenase (gpd)), PDC1, XR, XDH, transaldolase (TAL), transketolase (TKL), ribose 5-phosphate ketol-isomerase (RKI), CYB2, and the galactose family of genes (especially the GAL10 terminator). Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the yeast cell of choice may be used.

Suitable leaders for yeast host cells are obtained from the genes for enolase (e.g., S. cerevisiae or I. orientalis enolase (ENO-1)), 3-phosphoglycerate kinase (e.g., S. cerevisiae or I. orientalis 3-phosphoglycerate kinase), alpha-factor (e.g., S. cerevisiae or I. orientalis alpha-factor), and alcohol dehydrogenase/glyceraldehyde-3-phosphate dehydrogenase (e.g., S. cerevisiae or I. orientalis alcohol dehydrogenase/glyceraldehyde-3-phosphate dehydrogenase (ADH2/GAP)).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used. Useful polyadenylation sequences for yeast cells are described by Guo and Sherman, 1995, Mol. Cellular Biol. 15: 5983-5990.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. Potential integration loci include those described in the art (e.g., See US2012/0135481).

For autonomous replication, the vector may further comprise an origin of replication enabling the vector to replicate autonomously in the yeast cell. The origin of replication may be any plasmid replicator mediating autonomous replication that functions in a cell. The term “origin of replication” or “plasmid replicator” means a polynucleotide that enables a plasmid or vector to replicate in vivo. Examples of origins of replication for use in a yeast host cell are the 2 micron origin of replication, ARS1, ARS4, the combination of ARS1 and CEN3, and the combination of ARS4 and CEN6.

More than one copy of a polynucleotide described herein may be inserted into a host cell to increase production of a polypeptide. An increase in the copy number of the polynucleotide can be obtained by integrating at least one additional copy of the sequence into the yeast cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (see, e.g., Sambrook et al., 1989, supra).

Additional procedures and techniques known in the art for the preparation of recombinant cells for ethanol fermentation, are described in, e.g., WO 2016/045569, the content of which is hereby incorporated by reference.

### Processes of Ethanol Production

The recombinant cells described herein may be used for the production of ethanol. One aspect is a method for producing ethanol, comprising cultivating a recombinant cell described herein in a fermentable medium under suitable conditions to produce ethanol. In another aspect is a process for producing ethanol, comprising (a) saccharifying a cellulosic and/or starch-containing material with an enzyme composition; (b) fermenting the saccharified material of step (a) with any one of the recombinant cells described herein (e.g., a cell comprising a heterologous polynucleotide encoding a hexose transporter described herein and xylose isomerase). In one embodiment, the process comprises recovering the ethanol from the fermentation medium.

The processing of the cellulosic and/or starch containing material can be accomplished using methods conventional in the art. Moreover, the processes of can be implemented using any conventional biomass and/or starch processing apparatus configured to carry out the processes.

Saccharification (i.e., hydrolysis) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF).

SHF uses separate process steps to first enzymatically hydrolyze the cellulosic material to fermentable sugars, e.g., glucose, cellobiose, and pentose monomers, and then ferment the fermentable sugars to ethanol. In SSF, the enzymatic hydrolysis of the cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation organismcan tolerate. It is understood herein that any method known in the art comprising pretreatment, enzymatic hydrolysis (saccharification), fermentation, or a combination thereof, can be used in the practicing the processes described herein.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

### Cellulosic Pretreatment

In one embodiment the cellulosic material is pretreated before saccharification in step (a).

In practicing the processes described herein, any pretreatment process known in the art can be used to disrupt plant cell wall components of the cellulosic material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The cellulosic material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

In a one embodiment, the cellulosic material is pretreated before saccharification (i.e., hydrolysis) and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

In one embodiment, the cellulosic material is pretreated with steam. In steam pretreatment, the cellulosic material is heated to disrupt the plant cell wall components, including lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

In one embodiment, the cellulosic material is subjected to a chemical pretreatment. The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115). In a specific embodiment the dilute acid pretreatment of cellulosic material is carried out using 4% w/w sulfuric acid at 180° C. for 5 minutes.

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al, 2005, Appl. Biochem. Biotechnol 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one embodiment, the chemical pretreatment is carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt. % acid, e.g., 0.05 to 5 wt. % acid or 0.1 to 2 wt. % acid. The acid is contacted with the cellulosic material and held at a temperature in the range of preferably 140-200° C., e.g., 165-190° C., for periods ranging from 1 to 60 minutes.

In another embodiment, pretreatment takes place in an aqueous slurry. In preferred aspects, the cellulosic material is present during pretreatment in amounts preferably between 10-80 wt. %, e.g., 20-70 wt. % or 30-60 wt. %, such as around 40 wt. %. The pretreated cellulosic material can be unwashed or washed using any method known in the art, e.g., washed with water.

In one embodiment, the cellulosic material is subjected to mechanical or physical pretreatment. The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in one embodiment, the cellulosic material is subjected to physical (mechanical) or chemical pretreatment, or any combination thereof, to promote the separation and/or release of cellulose, hemicellulose, and/or lignin.

In one embodiment, the cellulosic material is subjected to a biological pretreatment. The term “biological pretreatment” refers to any biological pretreatment that promotes the separation and/or release of cellulose, hemicellulose, and/or lignin from the cellulosic material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

### Saccharification

In the saccharification step (i.e., hydrolysis step), the cellulosic and/or starch-containing material, e.g., pretreated, is hydrolyzed to break down cellulose, hemicellulose, and/or starch to fermentable sugars, such as glucose, cellobiose, xylose, xylulose, arabinose, mannose, galactose, and/or soluble oligosaccharides. The hydrolysis is performed enzymatically e.g., by a cellulolytic enzyme composition. The enzymes of the compositions can be added simultaneously or sequentially.

Enzymatic hydrolysis may be carried out in a suitable aqueous environment under conditions that can be readily determined by one skilled in the art. In one aspect, hydrolysis is performed under conditions suitable for the activity of the enzymes(s), i.e., optimal for the enzyme(s). The hydrolysis can be carried out as a fed batch or continuous process where the cellulosic and/or starch-containing material is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art. For example, the saccharification can last up to 200 hours, but is typically performed for preferably about 12 to about 120 hours, e.g., about 16 to about 72 hours or about 24 to about 48 hours. The temperature is in the range of preferably about 25° C. to about 70° C., e.g., about 30° C. to about 65° C., about 40° C. to about 60° C., or about 50° C. to about 55° C. The pH is in the range of preferably about 3 to about 8, e.g., about 3.5 to about 7, about 4 to about 6, or about 4.5 to about 5.5. The dry solids content is in the range of preferably about 5 to about 50 wt. %, e.g., about 10 to about 40 wt. % or about 20 to about 30 wt. %.

Saccharification in step (a) may be carried out using a cellulolytic enzyme composition. Such enzyme compositions are described below in the “Cellulolytic Enzyme Composition’-section below. The cellulolytic enzyme compositions can comprise any protein useful in degrading the cellulosic material. In one aspect, the cellulolytic enzyme composition comprises or further comprises one or more (e.g., several) proteins selected from the group consisting of a cellulase, an AA9 (GH61) polypeptide, a hemicellulase, an esterase, an expansin, a ligninolytic enzyme, an oxidoreductase, a pectinase, a protease, and a swollenin.

In another embodiment, the cellulase is preferably one or more (e.g., several) enzymes selected from the group consisting of an endoglucanase, a cellobiohydrolase, and a beta-glucosidase.

In another embodiment, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. In another embodiment, the oxidoreductase is one or more (e.g., several) enzymes selected from the group consisting of a catalase, a laccase, and a peroxidase.

The enzymes or enzyme compositions used in a processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

In one embodiment, an effective amount of cellulolytic or hemicellulolytic enzyme composition to the cellulosic material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the cellulosic material.

In one embodiment, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another aspect, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described in WO 2012/021401, and the soluble contents thereof. A liquor for cellulolytic enhancement of an AA9 polypeptide (GH61 polypeptide) can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic enzyme preparation. The liquor can be separated from the treated material using a method standard in the art, such as filtration, sedimentation, or centrifugation.

In one embodiment, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

### Cellulolytic Enzyme Composition

A cellulolytic enzyme composition may be present or added during saccharification in step (a). A cellulolytic enzyme composition is an enzyme preparation containing one or more (e.g., several) enzymes that hydrolyze cellulosic material. Such enzymes include endoglucanase, cellobiohydrolase, beta-glucosidase, and/or combinations thereof.

The cellulolytic enzyme composition may be of any origin. In an embodiment the cellulolytic enzyme composition is derived from a strain of Trichoderma, such as a strain of Trichoderma reesei; a strain of Humicola, such as a strain of Humicola insolens, and/or a strain of Chrysosporium, such as a strain of Chrysosporium lucknowense. In a preferred embodiment the cellulolytic enzyme preparation is derived from a strain of Trichoderma reesei.

The cellulolytic enzyme composition may further comprise one or more of the following polypeptides, such as enzymes: AA9 polypeptide (GH61 polypeptide) having cellulolytic enhancing activity, beta-glucosidase, xylanase, beta-xylosidase, CBH I, CBH II, or a mixture of two, three, four, five or six thereof.

The further polypeptide(s) (e.g., AA9 polypeptide) and/or enzyme(s) (e.g., beta-glucosidase, xylanase, beta-xylosidase, CBH I and/or CBH II may be foreign to the cellulolytic enzyme composition producing organism (e.g., Trichoderma reesei).

In an embodiment the cellulolytic enzyme preparation comprises an AA9 polypeptide having cellulolytic enhancing activity and a beta-glucosidase.

In another embodiment the cellulolytic enzyme preparation comprises an AA9 polypeptide having cellulolytic enhancing activity, a beta-glucosidase, and a CBH I.

In another embodiment the cellulolytic enzyme preparation comprises an AA9 polypeptide having cellulolytic enhancing activity, a beta-glucosidase, a CBH I and a CBH II.

Other enzymes, such as endoglucanases, may also be comprised in the cellulolytic enzyme composition.

As mentioned above the cellulolytic enzyme composition may comprise a number of difference polypeptides, including enzymes.

In one embodiment, the cellulolytic enzyme composition is a Trichoderma reesei cellulolytic enzyme composition, further comprising Thermoascus aurantiacus AA9 (GH61A) polypeptide having cellulolytic enhancing activity (e.g., WO 2005/074656), and Aspergillus oryzae beta-glucosidase fusion protein (e.g., one disclosed in WO 2008/057637, in particular shown as SEQ ID NOs: 59 and 60).

In another embodiment the cellulolytic enzyme composition is a Trichoderma reesei cellulolytic enzyme composition, further comprising Thermoascus aurantiacus AA9 (GH61A) polypeptide having cellulolytic enhancing activity (e.g., SEQ ID NO: 2 in WO 2005/074656), and Aspergillus fumigatus beta-glucosidase (e.g., SEQ ID NO: 2 of WO 2005/047499).

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO 2011/041397, and Aspergillus fumigatus beta-glucosidase (e.g., SEQ ID NO: 2 of WO 2005/047499).

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO 2011/041397, and Aspergillus fumigatus beta-glucosidase (e.g., SEQ ID NO: 2 of WO 2005/047499) or a variant disclosed in WO 2012/044915 (hereby incorporated by reference), in particular one comprising one or more such as all of the following substitutions: F100D, S283G, N456E, F512Y.

In an embodiment the cellulolytic enzyme composition is a Trichoderma reesei cellulolytic composition, further comprising an AA9 (GH61A) polypeptide having cellulolytic enhancing activity, in particular the one derived from a strain of Penicillium emersonii (e.g., SEQ ID NO: 2 in WO 2011/041397), Aspergillus fumigatus beta-glucosidase (e.g., SEQ ID NO: 2 in WO 2005/047499) variant with one or more, in particular all of the following substitutions: F100D, S283G, N456E, F512Y and disclosed in WO 2012/044915; Aspergillus fumigatus Cel7A CBH1, e.g., the one disclosed as SEQ ID NO: 6 in WO2011/057140 and Aspergillus fumigatus CBH II, e.g., the one disclosed as SEQ ID NO: 18 in WO 2011/057140.

In a preferred embodiment the cellulolytic enzyme composition is a Trichoderma reesei, cellulolytic enzyme composition, further comprising a hemicellulase or hemicellulolytic enzyme composition, such as an Aspergillus fumigatus xylanase and Aspergillus fumigatus beta-xylosidase.

In an embodiment the cellulolytic enzyme composition also comprises a xylanase (e.g., derived from a strain of the genus Aspergillus, in particular Aspergillus aculeatus or Aspergillus fumigatus; or a strain of the genus Talaromyces, in particular Talaromyces leycettanus) and/or a beta-xylosidase (e.g., derived from Aspergillus, in particular Aspergillus fumigatus, or a strain of Talaromyces, in particular Talaromyces emersonii).

In an embodiment the cellulolytic enzyme composition is a Trichoderma reesei cellulolytic enzyme composition, further comprising Thermoascus aurantiacus AA9 (GH61A) polypeptide having cellulolytic enhancing activity (e.g., WO 2005/074656), Aspergillus oryzae beta-glucosidase fusion protein (e.g., one disclosed in WO 2008/057637, in particular as SEQ ID NOs: 59 and 60), and Aspergillus aculeatus xylanase (e.g., Xyl II in WO 94/21785).

In another embodiment the cellulolytic enzyme preparation comprises a Trichoderma reesei cellulolytic preparation, further comprising Thermoascus aurantiacus GH61A polypeptide having cellulolytic enhancing activity (e.g., SEQ ID NO: 2 in WO 2005/074656), Aspergillus fumigatus beta-glucosidase (e.g., SEQ ID NO: 2 of WO 2005/047499) and Aspergillus aculeatus xylanase (Xyl II disclosed in WO 94/21785).

In another embodiment the cellulolytic enzyme composition comprises a Trichoderma reesei cellulolytic enzyme composition, further comprising Thermoascus aurantiacus AA9 (GH61A) polypeptide having cellulolytic enhancing activity (e.g., SEQ ID NO: 2 in WO 2005/074656), Aspergillus fumigatus beta-glucosidase (e.g., SEQ ID NO: 2 of WO 2005/047499) and Aspergillus aculeatus xylanase (e.g., Xyl II disclosed in WO 94/21785).

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO 2011/041397, Aspergillus fumigatus beta-glucosidase (e.g., SEQ ID NO: 2 of WO 2005/047499) and Aspergillus fumigatus xylanase (e.g., Xyl III in WO 2006/078256).

In another embodiment the cellulolytic enzyme composition comprises a Trichoderma reesei cellulolytic enzyme composition, further comprising Penicillium emersonii AA9 (GH61A) polypeptide having cellulolytic enhancing activity, in particular the one disclosed in WO 2011/041397, Aspergillus fumigatus beta-glucosidase (e.g., SEQ ID NO: 2 of WO 2005/047499), Aspergillus fumigatus xylanase (e.g., Xyl III in WO 2006/078256), and CBH I from Aspergillus fumigatus, in particular Cel7A CBH1 disclosed as SEQ ID NO: 2 in WO2011/057140.

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO 2011/041397, Aspergillus fumigatus beta-glucosidase (e.g., SEQ ID NO: 2 of WO 2005/047499), Aspergillus fumigatus xylanase (e.g., Xyl III in WO 2006/078256), CBH I from Aspergillus fumigatus, in particular Cel7A CBH1 disclosed as SEQ ID NO: 2 in WO 2011/057140, and CBH II derived from Aspergillus fumigatus in particular the one disclosed as SEQ ID NO: 4 in WO 2013/028928.

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO 2011/041397, Aspergillus fumigatus beta-glucosidase (e.g., SEQ ID NO: 2 of WO 2005/047499) or variant thereof with one or more, in particular all, of the following substitutions: F100D, S283G, N456E, F512Y; Aspergillus fumigatus xylanase (e.g., Xyl III in WO 2006/078256), CBH I from Aspergillus fumigatus, in particular Cel7A CBH I disclosed as SEQ ID NO: 2 in WO 2011/057140, and CBH II derived from Aspergillus fumigatus, in particular the one disclosed in WO 2013/028928.

In another embodiment the cellulolytic enzyme composition is a Trichoderma reesei cellulolytic enzyme composition comprising the CBH I (GENSEQP Accession No. AZY49536 (WO2012/103293); a CBH II (GENSEQP Accession No. AZY49446 (WO2012/103288); a beta-glucosidase variant (GENSEQP Accession No. AZU67153 (WO 2012/44915)), in particular with one or more, in particular all, of the following substitutions: F100D, S283G, N456E, F512Y; and AA9 (GH61 polypeptide) (GENSEQP Accession No. BAL61510 (WO 2013/028912)).

In another embodiment the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288); a GH10 xylanase (GENSEQP Accession No. BAK46118 (WO 2013/019827)); and a beta-xylosidase (GENSEQP Accession No. AZI04896 (WO 2011/057140)).

In another embodiment the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288)); and an AA9 (GH61 polypeptide; GENSEQP Accession No. BAL61510 (WO 2013/028912)).

In another embodiment the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288)), an AA9 (GH61 polypeptide; GENSEQP Accession No. BAL61510 (WO 2013/028912)), and a catalase (GENSEQP Accession No. BAC11005 (WO 2012/130120)).

In an embodiment the cellulolytic enzyme composition is a Trichoderma reesei cellulolytic enzyme composition comprising a CBH I (GENSEQP Accession No. AZY49446 (WO2012/103288); a CBH II (GENSEQP Accession No. AZY49446 (WO2012/103288)), a beta-glucosidase variant (GENSEQP Accession No. AZU67153 (WO 2012/44915)), with one or more, in particular all, of the following substitutions: F100D, S283G, N456E, F512Y; an AA9 (GH61 polypeptide; GENSEQP Accession No. BAL61510 (WO 2013/028912)), a GH10 xylanase (GENSEQP Accession No. BAK46118 (WO 2013/019827)), and a beta-xylosidase (GENSEQP Accession No. AZI04896 (WO 2011/057140)).

In an embodiment the cellulolytic composition is a Trichoderma reesei cellulolytic enzyme preparation comprising an EG I (Swissprot Accession No. P07981), EG II (EMBL Accession No. M19373), CBH I (supra); CBH II (supra); beta-glucosidase variant (supra) with the following substitutions: F100D, S283G, N456E, F512Y; an AA9 (GH61 polypeptide; supra), GH10 xylanase (supra); and beta-xylosidase (supra).

All cellulolytic enzyme compositions disclosed in WO 2013/028928 are also contemplated and hereby incorporated by reference.

The cellulolytic enzyme composition comprises or may further comprise one or more (several) proteins selected from the group consisting of a cellulase, a AA9 (i.e., GH61) polypeptide having cellulolytic enhancing activity, a hemicellulase, an expansin, an esterase, a laccase, a ligninolytic enzyme, a pectinase, a peroxidase, a protease, and a swollenin.

In one embodiment the cellulolytic enzyme composition is a commercial cellulolytic enzyme composition. Examples of commercial cellulolytic enzyme compositions suitable for use in a process of the invention include: CELLIC® CTec (Novozymes A/S), CELLIC® CTec2 (Novozymes A/S), CELLIC® CTec3 (Novozymes A/S), CELLUCLAST™ (Novozymes A/S), SPEZYME™ CP (Genencor Int.), ACCELLERASE™ 1000, ACCELLERASE 1500, ACCELLERASE™ TRIO (DuPont), FILTRASE® NL (DSM); METHAPLUS® S/L 100 (DSM), ROHAMENT™ 7069 W (Röhm GmbH), or ALTERNAFUEL® CMAX3™ (Dyadic International, Inc.). The cellulolytic enzyme composition may be added in an amount effective from about 0.001 to about 5.0 wt. % of solids, e.g., about 0.025 to about 4.0 wt. % of solids or about 0.005 to about 2.0 wt. % of solids.

Additional enzymes, and compositions thereof can be found in WO2016/0455569 (the content of which is incorporated herein in its entirety).

### Fermentation

The fermentable sugars obtained from the hydrolyzed cellulosic and/or starch-containing material can be fermented by one or more (e.g., several) fermenting microorganisms described herein capable of fermenting the sugars directly or indirectly into ethanol. “Fermentation” or “fermentation process” refers to any fermentation process or any process comprising a fermentation step.

In the fermentation step, sugars, released from the cellulosic and/or starch-containing material, e.g., as a result of the pretreatment and enzymatic hydrolysis steps, are fermented to ethanol, by a fermenting organism, such as yeast described herein. Hydrolysis (saccharification) and fermentation can be separate or simultaneous.

Any suitable hydrolyzed cellulosic and/or starch containing material can be used in the fermentation step in practicing the processes described herein. Such feedstocks include, but are not limited to carbohydrates (e.g., lignocellulose, xylans, cellulose, starch, etc.). The material is generally selected based on economics, i.e., costs per equivalent sugar potential, and recalcitrance to enzymatic conversion.

Production of ethanol by a fermenting microorganism using cellulosic material results from the metabolism of sugars (monosaccharides). The sugar composition of the hydrolyzed cellulosic material and the ability of the fermenting microorganism to utilize the different sugars has a direct impact in process yields. Prior to Applicant's disclosure herein, strains known in the art utilize glucose efficiently but do not (or very limitedly) metabolize pentoses like xylose, a monosaccharide commonly found in hydrolyzed material.

Compositions of the fermentation media and fermentation conditions depend on the fermenting organism and can easily be determined by one skilled in the art. Typically, the fermentation takes place under conditions known to be suitable for generating the fermentation product. In some embodiments, the fermentation process is carried out under aerobic or microaerophilic (i.e., where the concentration of oxygen is less than that in air), or anaerobic conditions. In some embodiments, fermentation is conducted under anaerobic conditions (i.e., no detectable oxygen), or less than about 5, about 2.5, or about 1 mmol/L/h oxygen. In the absence of oxygen, the NADH produced in glycolysis cannot be oxidized by oxidative phosphorylation. Under anaerobic conditions, pyruvate or a derivative thereof may be utilized by the host cell as an electron and hydrogen acceptor in order to generate NAD+.

The fermentation process is typically run at a temperature that is optimal for the recombinant fungal cell. For example, in some embodiments, the fermentation process is performed at a temperature in the range of from about 25° C. to about 42° C. Typically the process is carried out a temperature that is less than about 38° C., less than about 35° C., less than about 33° C., or less than about 38° C., but at least about 20° C., 22° C., or 25° C.

A fermentation stimulator can be used in a process described herein to further improve the fermentation, and in particular, the performance of the fermenting organism, such as, rate enhancement and product yield (e.g., ethanol yield). A “fermentation stimulator” refers to stimulators for growth of the fermenting organisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

The fermentation product, i.e., ethanol, can optionally be recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

In some aspects of the methods, the ethanol after being recovered is substantially pure. With respect to the methods of producing ethanol, “substantially pure” intends a recovered preparation that contains no more than 15% impurity, wherein impurity intends compounds other than ethanol. In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

In some embodiments of the methods described herein, fermentation of step (b) consumes an increased amount of glucose and pentose (e.g., xylose) when compared to fermentation using an identical cell without the heterologous polynucleotide encoding a hexose transporter under the same conditions (e.g., at about or after 40 hours fermentation, such as the conditions described in Example 3).

In one embodiment of the methods described herein, more than 65%, e.g., at least 70%, 75%, 80%, 85%, 90%, 95% of xylose in the medium is consumed at about or after 66 hours fermentation (e.g., under conditions described in Example 4).

In one embodiment of the methods described herein, more than 65%, e.g., at least 70%, 75%, 80%, 85%, 90%, 95% of glucose in the medium is consumed at about or after 66 hours fermentation (e.g., under conditions described in Example 4).

In one embodiment of the methods described herein, more than 65%, e.g., at least 70%, 75%, 80%, 85%, 90%, 95% of pentose (e.g., xylose) in the medium is consumed, and more than 65%, e.g., at least 70%, 75%, 80%, 85%, 90%, 95% of glucose in the medium is consumed, at about or after 66 hours fermentation (e.g., under conditions described in Example 4).

In some embodiments of the methods described herein, fermentation of step (b) provides higher ethanol yield when compared to fermentation using an identical cell without the heterologous polynucleotide encoding a hexose transporter under the same conditions (e.g., at about or after 40 hours fermentation, such as the conditions described in Example 3).

Suitable assays to test for the production of ethanol and contaminants, and sugar consumption can be performed using methods known in the art. For example, ethanol product, as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of ethanol in the fermentation broth can also be tested with the culture supernatant. Byproducts and residual sugar in the fermentation medium (e.g., glucose or xylose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

The invention may further be described in the following numbered paragraphs: Paragraph [1]. A recombinant yeast cell comprising a heterologous polynucleotide encoding a hexose transporter, wherein the transporter has at least 60%, e.g., at least 65%, 70%, 75%, 80%, 85%, 90%, 95%, 97%, 98%, 99%, or 100% sequence identity to SEQ ID NO: 2; and wherein the yeast cell is capable of fermenting xylose.

Paragraph [2]. The recombinant cell of paragraph [1], wherein the heterologous polynucleotide encodes a hexose transporter that differs by no more than ten amino acids, e.g., by no more than five amino acids, by no more than four amino acids, by no more than three amino acids, by no more than two amino acids, or by one amino acid from SEQ ID NO: 2.

Paragraph [3]. The recombinant cell of paragraph [1], wherein the heterologous polynucleotide encodes a hexose transporter having an amino acid sequence comprising or consisting of the amino acid sequence of SEQ ID NO: 2.

Paragraph [4]. The recombinant cell of any one of paragraphs [1]-[3], wherein the heterologous polynucleotide encoding a hexose transporter comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 1.

Paragraph [5]. The recombinant cell of paragraph [4], wherein the heterologous polynucleotide encoding a hexose transporter has a coding sequence that consists of SEQ ID NO: 1.

Paragraph [6]. The recombinant cell of any one of paragraphs [1]-[5], wherein the heterologous polynucleotide encoding a hexose transporter comprises a coding sequence that hybridizes under at least low stringency conditions e.g., medium stringency conditions, medium-high stringency conditions, high stringency conditions, or very high stringency conditions with the full-length complementary strand of SEQ ID NO: 1.

Paragraph [7]. The recombinant cell of any one of paragraphs claims [1]-[6], further comprising a heterologous polynucleotide encoding a xylose isomerase.

Paragraph [8]. The recombinant cell of paragraph [7], wherein the xylose isomerase has at least 60%, e.g., at least 65%, 70%, 75%, 80%, 85%, 90%, 95%, 97%, 98%, 99%, or 100% sequence identity to SEQ ID NO: 18.

Paragraph [9]. The recombinant cell of any one of paragraphs [1]-[8], wherein the strain has a higher anaerobic growth rate on a pentose (e.g., xylose) compared to the same cell without the heterologous polynucleotide encoding a hexose transporter at about or after 4 days of incubation (e.g., under conditions described in Example 2).

Paragraph [10]. The recombinant cell of any one of paragraphs [1]-[9], wherein the strain has a higher pentose (e.g., xylose) consumption compared to the same cell without the heterologous polynucleotide encoding a hexose transporter at about or after 40 hours fermentation (e.g., under conditions described in Example 3).

Paragraph [11]. The recombinant cell of any one of paragraphs [1]-[10], wherein the strain has a higher ethanol production compared to the same cell without the heterologous polynucleotide encoding a hexose transporter at about or after 40 hours fermentation (e.g., under conditions described in Example 3).

Paragraph [12]. The recombinant cell of any one of paragraphs [I]-[11], further comprising a heterologous polynucleotide encoding a xylulokinase (XK).

Paragraph [13]. The recombinant cell of paragraph [12] wherein the xylulokinase (XK) is a Saccharomyces cerevisiae XK, or an XK having at least 60%, e.g., at least 65%, 70%, 75%, 80%, 85%, 90%, 95%, 97%, 98%, 99%, or 100% sequence identity to SEQ ID NO: 22.

Paragraph [14]. The recombinant cell of any one of paragraphs [1]-[13], further comprising a heterologous polynucleotide encoding a ribulose 5 phosphate 3-epimerase (RPE1).

Paragraph [15]. The recombinant cell of paragraph [14], wherein the ribulose 5 phosphate 3-epimerase (RPE1) is a Saccharomyces cerevisiae RPE1, or an RPE1 having at least 60%, e.g., at least 65%, 70%, 75%, 80%, 85%, 90%, 95%, 97%, 98%, 99%, or 100% sequence identity to a Saccharomyces cerevisiae RPE1.

Paragraph [16]. The recombinant cell of any one of paragraphs [1]-[15], further comprising a heterologous polynucleotide encoding a ribulose 5 phosphate isomerase (RKI I).

Paragraph [17]. The recombinant cell of paragraph [16], wherein the ribulose 5 phosphate isomerase (RKI1) is a Saccharomyces cerevisiae RKI1, or an RKI1 having at least 60%, e.g., at least 65%, 70%, 75%, 80%, 85%, 90%, 95%, 97%, 98%, 99%, or 100% sequence identity to a Saccharomyces cerevisiae RKI1.

Paragraph [18]. The recombinant cell of any one of paragraphs [1]-[17], further comprising a heterologous polynucleotide encoding a transketolase (TKL1).

Paragraph [19]. The recombinant cell of paragraph [18], wherein the transketolase (TKL1) is a Saccharomyces cerevisiae TKL1, or an TKL1 having at least 60%, e.g., at least 65%, 70%, 75%, 80%, 85%, 90%, 95%, 97%, 98%, 99%, or 100% sequence identity to a Saccharomyces cerevisiae TKL1.

Paragraph [20]. The recombinant cell of any one of paragraphs [1]-[19], further comprising a heterologous polynucleotide encoding a transaldolase (TAL1).

Paragraph [21]. The recombinant cell of paragraph [20], wherein the transaldolase (TAL1) is a Saccharomyces cerevisiae TAL1, or an TAL1 having at least 60%, e.g., at least 65%, 70%, 75%, 80%, 85%, 90%, 95%, 97%, 98%, 99%, or 100% sequence identity to a Saccharomyces cerevisiae TAL1.

Paragraph [22]. The recombinant cell of any one of paragraphs [1]-[21], further comprise a disruption to an endogenous gene encoding a glycerol 3-phosphate dehydrogenase (GPD).

Paragraph [23]. The recombinant cell of any one of paragraphs [1]-[23], further comprise a disruption to an endogenous gene encoding a glycerol 3-phosphatase (GPP).

Paragraph [24]. The recombinant cell of any one of paragraphs [1]-[23], which is a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cell.

Paragraph [25]. The recombinant cell of any one of paragraphs aims [1]-[24], which is a Saccharomyces cerevisiae cell.

Paragraph [26]. The recombinant cell of paragraph [25], wherein the Saccharomyces cerevisiae is a derivative of a strain Saccharomyces cerevisiae CIBTS1260 (deposited under Accession No. NRRL Y-50973 at the Agricultural Research Service Culture Collection (NRRL), Illinois 61604 U.S.A.).

Paragraph [27]. The recombinant cell of any one of paragraphs [1]-[26], wherein the cell is capable of consuming more than 65%, e.g., at least 70%, 75%, 80%, 85%, 90%, 95% of pentose (e.g., xylose) in the medium at about or after 66 hours fermentation (e.g., under conditions described in Example 4).

Paragraph [28]. The recombinant cell of any one of paragraphs [1]-[27], wherein the cell is capable of consuming more than 65%, e.g., at least 70%, 75%, 80%, 85%, 90%, 95% of glucose in the medium at about or after 66 hours fermentation (e.g., under conditions described in Example 4).

Paragraph [29]. The recombinant cell of any one of paragraphs [1]-[28], wherein the cell is capable of consuming more than 65%, e.g., at least 70%, 75%, 80%, 85%, 90%, 95% of pentose (e.g., xylose) in the medium, and is capable of consuming more than 65%, e.g., at least 70%, 75%, 80%, 85%, 90%, 95% of glucose in the medium, at about or after 66 hours fermentation (e.g., under conditions described in Example 4).

Paragraph [30]. A process for producing ethanol, comprising cultivating the recombinant cell of any one of paragraphs [1]-[29] in a fermentable medium under suitable conditions to produce ethanol.

Paragraph [31]. The process of paragraph [30], wherein cultivation is conducted under low oxygen (e.g., anaerobic) conditions.

Paragraph [32]. The process of paragraph [31] or [32], wherein an increased amount of glucose and pentose (e.g., xylose) is consumed when compared to the process using an identical cell without the heterologous polynucleotide encoding a hexose transporter under the same conditions (e.g., at about or after 40 hours fermentation, such as the conditions described in Example 3).

Paragraph [33]. The process of any one of paragraphs [30]-[32], wherein more than 65%, e.g., at least 70%, 75%, 80%, 85%, 90%, 95% of pentose (e.g., xylose) in the medium is consumed, and more than 65%, e.g., at least 70%, 75%, 80%, 85%, 90%, 95% of glucose in the medium is consumed, at about or after 66 hours fermentation (e.g., under conditions described in Example 4).

Paragraph [34]. The process of any one of paragraphs [30]-[33], wherein the process results in higher ethanol yield when compared to the process using an identical cell without the heterologous polynucleotide encoding a hexose transporter under the same conditions (e.g., at about or after 40 hours fermentation, such as the conditions described in Example 3).

Paragraph [35]. The process of any one of paragraphs [30]-[34], comprising recovering the fermentation product from the fermentation.

Paragraph [36]. The process of any one of paragraphs [30]-[35], comprising saccharifying a cellulosic and/or starch-containing material with an enzyme composition to produce the fermentable medium.

Paragraph [37]. The process of paragraph [36], wherein saccharification occurs on a cellulosic material, and wherein the cellulosic material is pretreated.

Paragraph [38]. The process of paragraph [37], wherein the pretreatment is a dilute acid pretreatment.

Paragraph [39]. The process of any one of paragraphs [36]-[38], wherein saccharification occurs on a cellulosic material, and wherein the enzyme composition comprises one or more enzymes selected from a cellulase, an AA9 polypeptide, a hemicellulase, a CIP, an esterase, an expansin, a ligninolytic enzyme, an oxidoreductase, a pectinase, a protease, and a swollenin.

Paragraph [40]. The process of paragraph [39], wherein the cellulase is one or more enzymes selected from an endoglucanase, a cellobiohydrolase, and a beta-glucosidase.

Paragraph [41]. The process of paragraph [39] or [40], wherein the hemicellulase is one or more enzymes selected a xylanase, an acetylxylan esterase, a feruloyl esterase, an arabinofuranosidase, a xylosidase, and a glucuronidase.

Paragraph [42]. The process of any of paragraphs [36]-[41], wherein fermentation and saccharification are performed simultaneously in a simultaneous saccharification and fermentation (SSF).

Paragraph [43]. The process of any of paragraphs [36]-[41], wherein fermentation and saccharification are performed sequentially (SHF).

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### EXAMPLES

Chemicals used as buffers and substrates were commercial products of at least reagent grade.

### Strains

Ethanol Red® is an industrial S. cerevisiae strain used throughout the biofuel industry and was sporulated according to the method of Herskowitz (1988) to generate haploids. One of the haploids, YGT40, was used as a template for PCR amplification of the TEF1 promoter (P_{TEF1}-Sc), hxt2 gene and TIP1 terminator (T_{TIP1}) sequences.

Strain S. cerevisiae JG169 (WO2008/008967) was used as a template for amplification of the left and right flanks in plasmid pFYD1092.

Strain S. cerevisiae FYD853 (See WO2016/045569, strain CIBTS1260) is an engineered strain expressing xylose isomerase used as a template for amplification of the left and right flanks in plasmid pFYD1497 and as host for HXT2 expression.

Strain S. cerevisiae CIBTS1260 was deposited by Novozymes A/S under the terms of the Budapest Treaty with the Agricultural Research Service Culture Collection (NRRL), 1815 North University Street, Peoria, Ill. 61604 U.S.A.) and given the following accession number:

| Deposit | Accession Number | Date of Deposit |
|---|---|---|
| CIBTS1260 | NRRL Y-50973 | Sep. 5, 2014 |

Strain S. cerevisiae MBG4982 was produced from S. cerevisiae FYD853 using methods similar to those described in WO2005/121337 by mating haploids capable of growing anaerobically on xylose minimal medium with complementary haploids derived from a long term culture of yeast selected for their resistance to inhibitors found in cellulosic hydrolysates. Hybrid strains were sporulated and haploids germinated on xylose minimal medium under anaerobic conditions. A mass mated culture was produced from these haploids and subject to several rounds of selection for the ability to grow on xylose and the ability to tolerate hydrolysates. After selection, MBG4982 was identified based on ability to ferment xylose and tolerance to inhibitors in hydrolysate medium.

### Media and Solutions

LB+amp medium was composed of 10 g tryptone, 5 g yeast extract, 10 g NaCl, deionized water to 1 L and 100 mg/I ampicillin. For LB+amp agar plates, 15 g/L bacto agar was used and the concentration of ampicillin was increased to 150 mg/L.

YPD medium was composed of 10 g yeast extract, 20 g peptone, 20 g glucose and deionized water to 1 L. For plates, 20 g/I bacto agar was used and hygromycin B was added to 200 mg/L where appropriate.

2xYPD medium was composed of 20 g yeast extract, 40 g peptone, 40 g glucose and deionized water to 1 L.

1 M K₂HPO₄ buffer was composed of 228.23 g K₂HPO₄×3 H₂O and deionized water to 1 L.

1 M KH₂PO₄ buffer was composed of 136.09 g KH₂PO₄ and deionized water to 1 L.

1 M phosphate buffer (pH=6.0) was composed of 132 mL of 1 M K₂HPO₄ and 868 mL of 1 M of KH₂PO₄.

SD2 medium was composed of 6.7 g yeast nitrogen base without amino acids, 100 mL 1 M phosphate buffer (pH=6.0), 20 g glucose and deionized water to 1 L. For plates, 20 g/L bacto agar was added.

SX2 medium was composed of 6.7 g yeast nitrogen base without amino acids, 100 mL 1 M phosphate buffer (pH=6.0), 20 g xylose (BioUltra, Sigma-Aldrich) and deionized water to 1 L. For plates, 20 g/L bacto agar was added.

SX2.5 medium was composed of 6.7 g yeast nitrogen base without amino acids, 100 mL 1 M phosphate buffer (pH=6.0), 25 g xylose (BioUltra, Sigma-Aldrich) and deionized water to 1 L.

SD5X2.5 medium was composed of 6.7 g yeast nitrogen base without amino acids, 100 mL 1 M phosphate buffer (pH=6.0), 50 g glucose, 25 g xylose (BioUltra, Sigma-Aldrich) and deionized water to 1 L.

SX1/SD1 medium was composed 6.7 g yeast nitrogen base without amino acids, 100 mL 1 M phosphate buffer (pH=6.0), 10 g glucose, 10 g xylose (BioUltra, Sigma-Aldrich) and deionized water to 1 L.

SX6 medium was composed 6.7 g yeast nitrogen base without amino acids, 100 mL 1 M phosphate buffer (pH=6.0), 60 g xylose (BioUltra, Sigma-Aldrich) and deionized water to 1 L.

SD6 medium was composed 6.7 g yeast nitrogen base without amino acids, 100 mL 1 M phosphate buffer (pH=6.0), 60 g glucose (BioUltra, Sigma-Aldrich) and deionized water to 1 L.

SX3/SD3 medium was composed 6.7 g yeast nitrogen base without amino acids, 100 mL 1 M phosphate buffer (pH=6.0), 30 g glucose, 30 g xylose (BioUltra, Sigma-Aldrich) and deionized water to 1 L.

TBE buffer was composed of 10.8 g of Tris base, 5.5 g of boric acid, 4 mL of 0.5 M EDTA pH=8.0, and deionized water to 1 L.

TABLE 1
Primer sequences

| Identifier | Sequence (5′→3′) | SEQ ID NO |
|---|---|---|
| OY474 | GGTTGTTTATGTTCGGATGTGATG | 3 |
| OY796 | ACATTATACGAAGTTATTTAATTAACATATAATACAT | 4 |
| | ATCACATAGGAAGC | |
| OY1476 | ATTCAGACATTTTGTAATTAAAACTTAGATTAGATT | 5 |
| | GCTATGC | |
| OY1477 | TAATTACAAAATGTCTGAATTCGCTACTAGCG | 6 |
| OY1478 | AGGTTCCCTTTTATTCCTCGGAAACTCTTTTTTCTT | 7 |
| | TTGAG | |
| OY1479 | CGAGGAATAAAAGGGAACCTTTTACAACAAATATT | 8 |
| | TG | |
| OY1481 | CGTCAAGGCCGCATGCGGCCGCGGAATAGTGAC | 9 |
| | GTTGTGATGC | |
| OY1482 | TGCTATACGAAGTTATGTTTAAACCTAAACTAACAT | 10 |
| | CGCGATGC | |
| OY1483 | AATATGGGCGCGATCGCTAAGTACAGACGGAAAC | 11 |
| | TCACACC | |
| OY1484 | CCCATGAGGCCCAGGGCGCGCCCTACAGATGTTG | 12 |
| | CTGCAACC | |
| OY1485 | TTAGCGATCGCGCCCATATTTAGCTCGTTTGG | 13 |
| OY1492 | GCTGGCTACTCGTTGCTCG | 14 |
| OY2357 | TGCTATACGAAGTTATGTTTAAACCTAAACTAACAT | 15 |
| | CGCATTGC | |
| OY2394 | TGATCTGCAGTAGAATCAGTGG | 16 |
| OY2395 | ACTTGTGTGGATGCCAACG | 17 |
| 1222569 | GGGCCCTCCTTACTGCTC | 23 |
| 1222570 | TAGACGCAGTACAAGGACGC | 24 |
| XII-2 | TGCAATTCAATAAATGGGATGTGATTG | 25 |
| external forward | | |
| XII-2 | GAGCGAACGTAAGAGAGGTTAATGTCCTCTAAC | 26 |
| external reverse | | |
| 1220142 | GATGATCGAGCCGGTAGTTAAC | 27 |
| 1222570 | TAGACGCAGTACAAGGACGC | 28 |
| 1221575 | AGCACAATCCAAGGAAAAATCTGGCC | 30 |
| 1221475 | GCCATTAGTAGTGTACTCAAACGAATTATTGTTG | 31 |
| 1221471 | TCAGTACTGACAATAAAAAGATTCTTGTTTTCAAGA | 32 |
| | ACTT | |
| 1221756 | TAGCGTGTTACGCACCCAAAC | 33 |
| 1221473 | ACAGAAGACGGGAGACACTAGC | 34 |
| 1221470 | TTTGTTTGTTTATGTGTGTTTATTCGAAACTAAGTT | 35 |
| | CT | |
| 1221746 | AGTTGATTGTATGCTTGGTATAGCTTGAAATATTG | 36 |
| 1221754 | TGTTTTATATTTGTTGTAAAAAGTAGATAATTACTTC | 37 |
| | CTTGATGATCTG | |
| 1221472 | TTTGTTTTTTGTTTTCTTCTAATTGATTTTTTCTTTCT | 38 |
| | ATTTCCTTTG | |
| 1221747 | GGGGTCGCAACTTTTCCC | 39 |

### HPLC Protocol

The content of acetate, glucose, xylose, glycerol and ethanol was determined by means of an Alliance 2695 HPLC (Waters Corp.) with Waters 2414 RI detector (Waters Corp.) and controlled by Empower™ 3 software (Waters Corp.). Instrument settings are listed in Table 2 below.

TABLE 2
HPLC instrument settings used to analyze
samples in the following examples.

| | |
|---|---|
| Column | Rezex ROA-Organic Acid H+ |
| Column dimensions | 300 × 7.8 mm |
| Particle size | 8 μm |
| Guard | SecurityGuard ™ Carbo-H+ |
| Guard dimensions | 4 × 3.0 mm |
| Manufacturer | Phenomenex |
| Column temp. | 60° C. |
| Flow rate | 0.7 mL/min |
| Mobile phase | 5 mM sulfuric acid |
| Elution | Isocratic |
| Injection volume | 10 μL |
| Detection | RI, 35° C. |
| Run time | 25 min. |

### Example 1: Construction of Yeast Strain FYD1547

This example describes the construction of yeast strain FYD1547 expressing a xylose isomerase, also containing one copy of the hxt2 gene (comprising the coding sequence of SEQ ID NO: 1, encoding the hexose transporter 2 of SEQ ID NO: 2), under the control of the TEF1 promoter (P_{TEF1}-Sc) integrated at the CHR XI-1 locus in the FYD1547 genome.

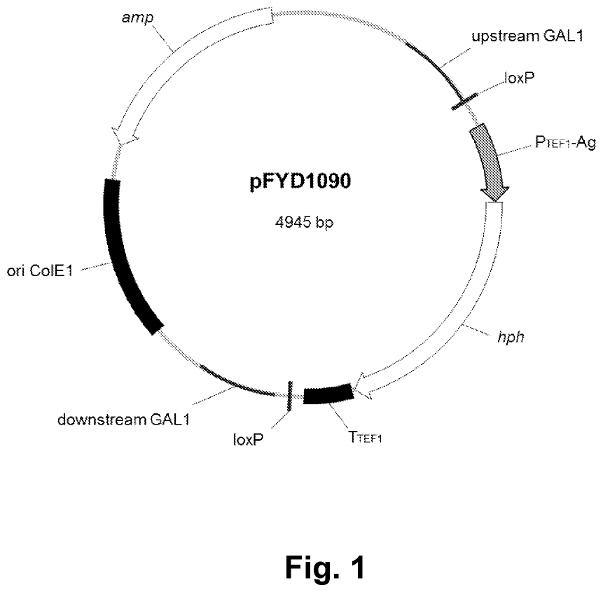

Plasmid pFYD1090 (FIG. 1; SEQ ID NO: 19), containing a hygromycin resistance marker (hph) and flanks for disruption of the GAL1 gene, was ordered as a synthetic gene from GeneArt (Thermo Fisher Scientific).

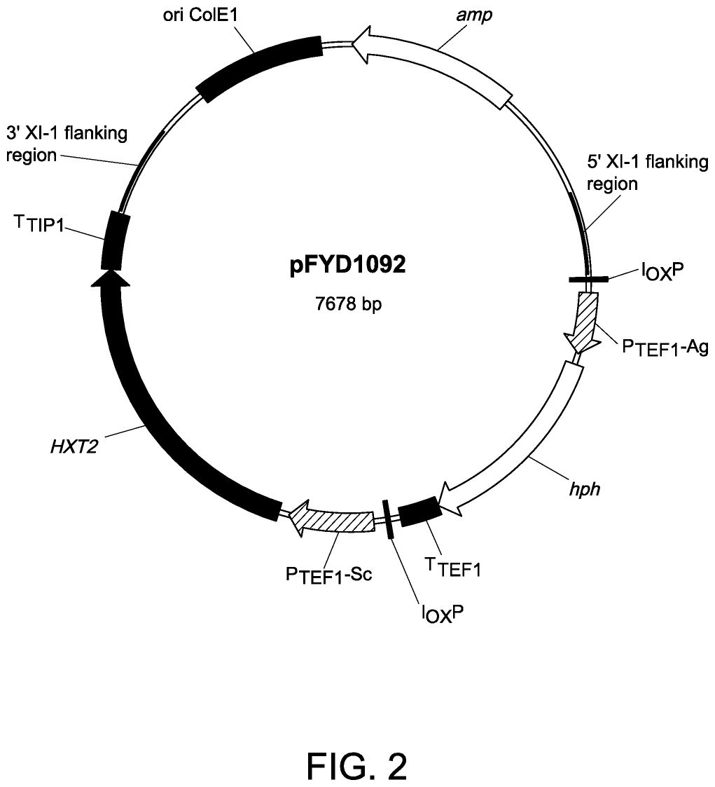

The first cloning step was to replace the “upstream GAL1” and the “downstream GAL1” homology regions in pFYD1090 (FIG. 1; SEQ ID NO: 19) with the 5′ CHR XI-1 and 3′ CHR XI-1 flanking regions in pFYD1090 and insert the TEF1 promoter (P_{TEF1}-Sc), hxt2 gene and TIP1 terminator (T_{TIP1}) to generate pFYD1092. The GAL1 upstream and downstream homology regions along with additional restriction sites and flanking DNA for cloning were amplified with the primer sets OY1481+OY1482 and OY1483+OY1484, respectively. The amplification reactions were performed using Phusion® Hot Start Flex DNA Polymerase (New England Biolabs) according to the manufacturer's instructions. Each PCR for the GAL1 upstream and downstream regions was composed of 1 μL of a JG169 genomic DNA preparation, 1×HF buffer, 200 μM of each dNTP, 500 nM forward primer, 500 nM reverse primer and 1 unit of Phusion® Hot Start Flex DNA Polymerase. The TEF1 promoter (P_{TEF1}-Sc), hxt2 gene and TIP1 terminator (T_{TIP1}) along with additional flanking DNA for cloning were amplified with the primer sets OY796+OY1476, OY1477+OY1478 and OY1479+OY1485, respectively. The amplification reactions were performed using Phusion® Hot Start Flex DNA Polymerase (New England Biolabs) according to the manufacturer's instructions. Each PCR was composed of 1 μL of a YGT40 genomic DNA preparation, 1×HF buffer, 200 μM of each dNTP, 500 nM forward primer, 500 nM reverse primer and 1 unit of Phusion® Hot Start Flex DNA Polymerase. The reactions were incubated in a Bio-Rad C1000 Touch™ Thermal Cycler (Bio-Rad Laboratories) programmed for 1 cycle at 98° C. for 3 minutes; 35 cycles each at 98° C. for 10 seconds, 55° C. for 30 seconds and 72° C. for 1.5 minutes; and one cycle at 72° C. for 5 minutes. Following thermocycling, the PCR products were separated by 1% agarose gel electrophoresis in TBE buffer and the bands (569 bp, 603 bp, 557 bp, 1646 bp and 331 bp) corresponding to the different PCR products (GAL1 upstream, GAL1 downstream, P_{TEF1}-Sc, hxt2 and T_{TIP1}, respectively) were excised from the gel and purified using an illustra GFX PCR DNA and Gel Band Purification Kit (GE Healthcare Life Sciences) according to the manufacturer's instructions. Plasmid pFYD1090 was digested with AscI/NotI-HF/PacI/PmeI and the restriction enzyme digestion bands were separated by 1% agarose gel electrophoresis in TBE buffer. The 2349 bp AscI/NotI fragment corresponding to the plasmid backbone and the 1781 bp PacI/PmeI fragment corresponding to the hygromycin resistance cassette were excised from the gel and purified using an illustra GFX PCR DNA and Gel Band Purification Kit (GE Healthcare Life Sciences) according to the manufacturer's instructions. The two restriction enzyme digestion fragments and the PCR products were joined together using an In-Fusion® HD EcoDry™ Cloning Kit (Clontech Laboratories, Inc.) in a total volume of 10 μL composed of 45 ng of the 2349 bp AscI/NotI pFYD1090 fragment, 68 ng of the 1781 bp PacI/PmeI fragment, 21 ng P_{TEF1}-Sc PCR product, 63 ng of hxt2 PCR product and 13 ng of T_{TIP1} PCR product. The reaction was incubated at 37° C. for 15 minutes, 50° C. for 15 minutes and then placed on ice. The reaction was used to transform Stellar™ Competent Cells (Clontech Laboratories, Inc.) according to the manufacturer's instructions. The transformation reaction was spread onto two LB+amp plates and incubated at 37° C. overnight. Putative transformant colonies were isolated from the selection plates and plasmid DNA was prepared from each one using a QIAprep 96 Turbo Kit (Qiagen) and screened for proper insertion of the fragments by digestion with PvuII. A plasmid yielding the desired band sizes was confirmed to be correct by DNA sequencing and designated pFYD1092 (FIG. 2; SEQ ID NO: 20).

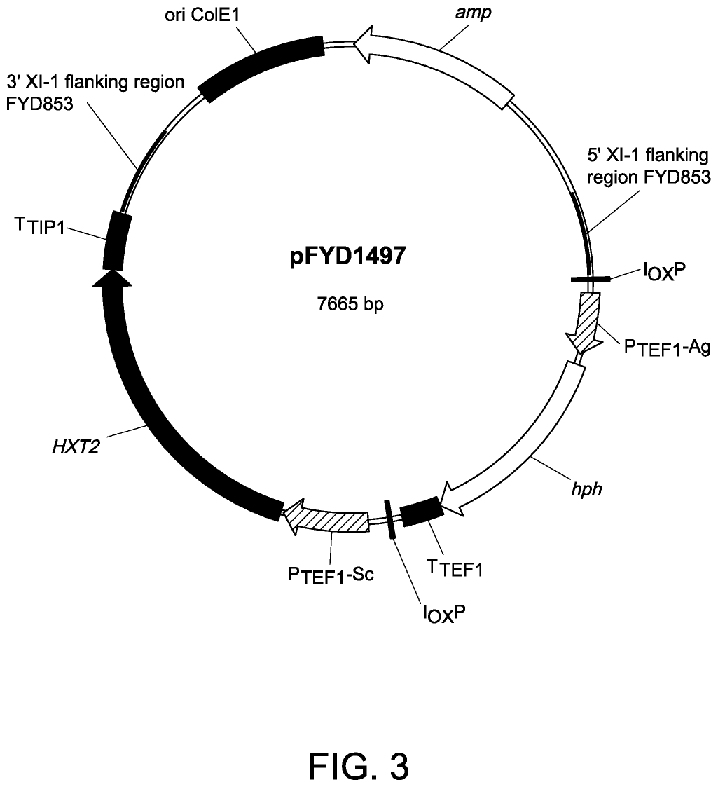

To ensure correct integration of the hxt2 expression cassette into FYD853, plasmid pFYD1497 (FIG. 3; SEQ ID NO: 21) was prepared by exchanging the 5′ CHR XI-1 and 3′ CHR XI-1 flanking regions from JG169 in pFYD1092 (supra) with the corresponding regions from FYD853. The 5′ CHR XI-1 and 3′ CHR XI-1 flanking regions along with additional restriction sites and flanking DNA for cloning were amplified with the primer sets OY1481+OY2357 and OY1483+OY1484, respectively. The amplification reactions were performed using Phusion® Hot Start Flex DNA Polymerase (New England Biolabs) according to the manufacturer's instructions. Each PCR for the 5′ CHR XI-1 and 3′ CHR XI-1 flanking regions was composed of 1 μL of a FYD853 genomic DNA preparation, 1×HF buffer, 200 μM of each dNTP, 500 nM forward primer, 500 nM reverse primer and 1 unit of Phusion® Hot Start Flex DNA Polymerase. The reactions were incubated in a Bio-Rad C1000 Touch™ Thermal Cycler (Bio-Rad Laboratories) programmed for 1 cycle at 98° C. for 30 seconds; 30 cycles each at 98° C. for 10 seconds, 55° C. for 30 seconds and 72° C. for 2 minutes; and one cycle at 72° C. for 10 minutes. Following thermocycling, the PCR products were separated by 1% agarose gel electrophoresis in TBE buffer and the bands (561 bp and 598 bp) corresponding to the 5′ CHR XI-1 and 3′ CHR XI-1 flanking regions were excised from the gel and purified using an illustra GFX PCR DNA and Gel Band Purification Kit (GE Healthcare Life Sciences) according to the manufacturer's instructions. Plasmid pFYD1092 was digested with AscI/AsiSI/NotI-HF/PmeI and the restriction enzyme digestion bands were separated by 1% agarose gel electrophoresis in TBE buffer. The 2349 bp AscI/NotI fragment corresponding to the plasmid backbone and the 4227 bp AsiSI/PmeI fragment corresponding to the hygromycin resistance cassette and the hxt2 expression cassette were excised from the gel and purified using an illustra GFX PCR DNA and Gel Band Purification Kit (GE Healthcare Life Sciences) according to the manufacturer's instructions. The two restriction enzyme digestion fragments and the PCR products were joined together using an In-Fusion® HD EcoDry™ Cloning Kit (Clontech Laboratories, Inc.) in a total volume of 10 μL composed of 57 ng of the 2349 bp AscI/NotI pFYD1092 fragment, 207 ng of the 4227 bp AsiSI/PmeI fragment, 27 ng of the 5′ CHR XI-1 PCR product and 29 ng of the 3′ CHR XI-1 PCR product. The reaction was incubated at 37° C. for 15 minutes, 50° C. for 15 minutes and then placed on ice. The reaction was used to transform Stellar™ Competent Cells (Clontech Laboratories, Inc.) according to the manufacturer's instructions. The transformation reaction was spread onto two LB+amp plates and incubated at 37° C. overnight. Putative transformant colonies were isolated from the selection plates and plasmid DNA was prepared from each one using a QIAprep 96 Turbo Kit (Qiagen) and screened for proper insertion of the fragments by digestion with HindIII-HF/PmII. A plasmid harboring the desired band sizes was confirmed to be correct by DNA sequencing and designated pFYD1497 (FIG. 3; SEQ ID NO: 21).

Competent FYD853 cells were prepared according to the protocol described in Gietz & Schiestl (2008) except that 2xYPD media was used instead of 2xYPAD media. The pFYD1497 plasmid was digested with NotI-HF and AscI and approx. 2 μg DNA was used to transform competent FYD853 cells. Following transformation, cells were pelleted at 15,000×g for 30 seconds. Cells were resuspended in 1 mL 2xYPD media and incubated at 30° C. in a thermomixer with shaking for 4.5 hours. Cells were spread onto YPD+200 μg/mL hygromycin plates (200 μL cells were spread per plate) and incubated at 30° C. for two days. Putative transformants were streaked on new YPD+200 μg/mL hygromycin plates and incubated at 30° C. for three days. The transformants were screened for correct integration of the hxt2 expression cassette by PCR using primer sets OY2394+OY474 (verification of 5′ CHR XI-1 flank) and OY1492+OY2395 (verification of 3′ CHR XI-1 flank). The amplification reactions were performed using a Phire Plant Direct PCR Master Mix (Thermo Fisher Scientific) according to the manufacturer's instructions and correct transformants should produce a 1094 bp band for the OY2394+OY474 5′ CHR XI-1 PCR and a 795 bp band for the OY1492+OY2395 3′ CHR XI-1 PCR. Four transformants with the desired amplicon sizes were saved for future testing and referenced as FYD1547#1-4.

### Example 2: Evaluation of Anaerobic Growth of Genetically Engineered Strains Comprising a Heterologous Polynucleotide Expressing HXT2

In order to test xylose utilization and anaerobic growth, the FYD853 strain and the FYD1547 transformants (#1-4) from Example 1 were streaked on fresh YPD agar plates and incubated at 30° C. for 2 days. Independent 5 mL YPD cultures were prepared for 4 colonies from FYD853 and for 1 colony from each of the FYD1547 strain candidates and incubated overnight with shaking at 30° C. Next day, cells from 1 mL YPD overnight culture were collected by centrifugation (7,000×g for 3 minutes) and re-suspended in 1 mL SX2.5 media. The optical density at 600 nm (OD_{600 nm}) was recorded for each of the cell suspensions and 15 mL SX2.5 media was inoculated to OD_{600 nm}=0.1. Ten-fold serial dilutions were prepared from each of the cell suspensions down to OD_{600 nm}=0.0001 (1,000× dilution) and 2 μL of each dilution incl. the OD_{600 nm}=0.1 stock was spotted onto SD2 and SX2 agar plates. Once the liquid had been absorbed the plates were placed in sealed plastic containers together with Oxoid™ AnaeroGen™ 2.5 L sachets and Oxoid™ Resazurin anaerobic indicators (Thermo Scientific, Oxoid Microbiology Products) and incubated at 30° C. The containers were inspected every day to ensure that the conditions remained anaerobic. Pictures were taken of the plates on days 4, 5, 6 and 7. Once the pictures were taken the plates were immediately placed in the plastic containers and new Oxoid™ AnaeroGen™ 2.5 L sachets and Oxoid™ Resazurin anaerobic indicators (Thermo Scientific, Oxoid Microbiology Products) were added and incubation was resumed at 30° C.

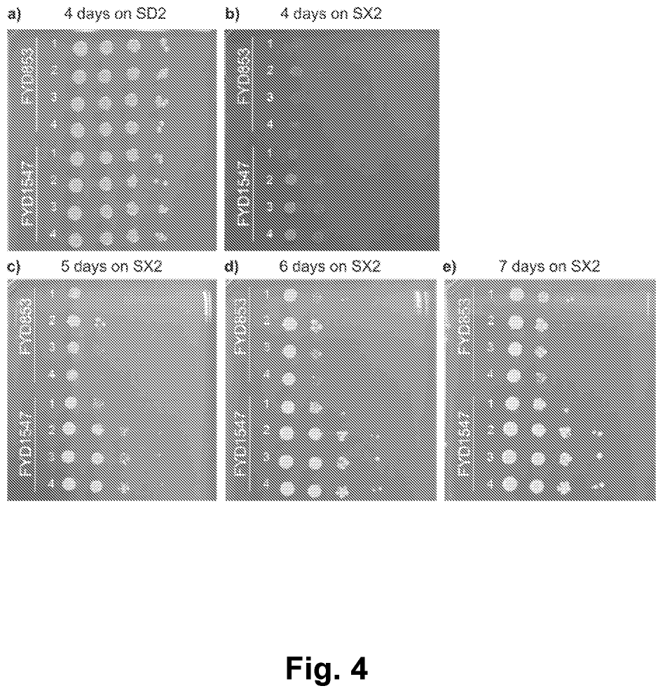

As shown in FIG. 4, the constitutive expression of the HXT2 transporter in FYD1547 greatly increased the xylose utilization and growth of the FYD1547 isolates compared to the parental FYD853 strain (lacking the hxt2 expression cassette). By day 5, the FYD1547 isolates demonstrated significant growth while the FYD853 showed very little growth. On day 7, three of the FYD1547 isolates had formed large colonies on all the spots of the dilution series whereas the FYD853 strain generally only showed visible growth on the spots from the two first dilutions.

### Example 3: Evaluation of Anaerobic Xylose Utilization from Genetically Engineered Strains Comprising a Heterologous Polynucleotide Expressing HXT2

To test how constitutive expression of the HXT2 transporter might affect xylose utilization in liquid culture under anaerobic conditions, the FYD853 strain and the FYD1547 isolates (#1-4) were streaked on fresh YPD agar plates and incubated at 30° C. for 2 days. Independent 5 mL YPD cultures were prepared for 4 colonies from FYD853 and for 1 colony from each FYD1547 isolate and incubated overnight with shaking at 30° C. Next day, cells from 1 mL YPD overnight culture were collected by centrifugation (7,000×g for 3 minutes) and resuspended in 1 mL SX2.5 media. The optical density at 600 nm (OD_{600 nm}) was recorded for each of the cell suspensions and 15 mL SX2.5 media was inoculated to OD_{600 nm}=0.1. For each isolate, 4 BD Plastipak™ Plastic Concentric Luer-Lock 50 ml syringes (Fisher Scientific) containing 3 mL SX2.5 cell suspension were prepared. Prior to each inoculation, the piston of each syringe was removed and 3 mL SX2.5 cell suspension was added. The piston was then carefully reinserted and residual air was removed by pressing the plunger until a meniscus of liquid was visible at the tip of the syringe. The syringe was then sealed with a BD™ Combi™ Luer-Lock plug (Fisher Scientific). The inoculated syringes were incubated at 30° C. with 200 rpm shaking. Samples were taken for HPLC analysis at the start of the experiment (0 hours) and after 40.6 hours, 52.9 hours and 67.6 hours. At the designated time points, the cultivation broth was filtered through a 0.22 μm Millex-GP Med Syringe Filter Unit with a PES membrane (Merck Millipore) and collected in a 1.5 mL Eppendorf tube. For HPLC analysis, the filtered culture broth was mixed in a 1:1 ratio with 5 mM H₂SO₄ and sent for HPLC analysis at the department for Analytical Development at Novozymes A/S, Denmark.

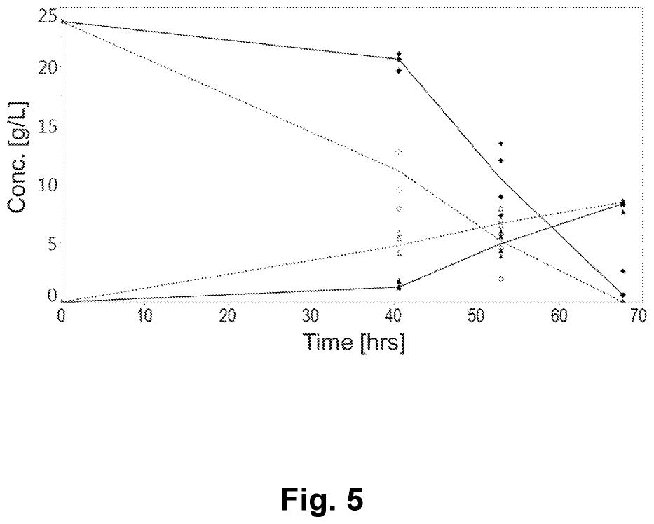

The results from the HPLC analysis are shown in Table 3 below and graphically in FIG. 5. Constitutive expression of the HXT2 transporter in FYD1547 greatly improved the xylose consumption and ethanol (EtOH) production as more than 50% of the xylose was consumed by the FYD1547 isolates by 40.6 hours compared to approx. 5% consumed for the FYD853 strain lacking the constitutively expressed HXT2 transporter. The FYD1547 isolates had consumed all xylose by the end of the experiment whereas 0.6 g/L xylose (median value) was still left in the FYD853 fermentations.

TABLE 3
Anaerobic syringe fermentations in SX2.5 media.

| | | Time [hours] | | | |
|---|---|---|---|---|---|
| Strain | | 0 | 40.6 | 52.9 | 67.6 |
| FYD853 | Xylose [g/L] | 23.80 | 20.57 | 10.44 | 0.60 |
| | EtOH [g/L] | 0** | 1.27 | 4.95 | 8.34 |
| | EtOH yield [g/g]* | — | — | — | 0.36 |
| FYD1547 | Xylose [g/L] | 23.80 | 11.11 | 5.17 | 0*** |
| | EtOH [g/L] | 0** | 4.78 | 6.69 | 8.47 |
| | EtOH yield [g/g]* | — | — | — | 0.36 |

*EtOH yield is calculated as g EtOH per g xylose consumed.
**No EtOH was detected.
***No xylose was detected.

### Example 4: Evaluation of Anaerobic Xylose and Glucose Utilization from Genetically Engineered Strains Comprising a Heterologous Polynucleotide Expressing HXT2

To test how constitutive expression of the HXT2 transporter might affect xylose utilization and fermentation performance in a liquid media containing glucose and xylose in approximately the same ratio as pre-treated corn stover (PCS), a synthetic media containing 50 g/L glucose and 25 g/L xylose was prepared (corresponding to approx. 11% total solids NREL PCS). The FYD853 strain and the FYD1547 isolates (#1-4) were streaked on fresh YPD agar plates and incubated at 30° C. for 2 days. Independent 5 mL YPD cultures were prepared for 4 colonies from FYD853 and for 1 colony from each FYD1547 isolate and incubated overnight with shaking at 30° C. Next day, cells from 1 mL YPD overnight culture were collected by centrifugation (7,000×g for 3 minutes) and re-suspended in 1 mL SD5X2.5 media. The optical density at 600 nm (OD_{600 nm}) was recorded for each of the cell suspensions and 10 mL SD5X2.5 media was inoculated to OD_{600 nm}=0.1. For each isolate, 4 BD Plastipak™ Plastic Concentric Luer-Lock 50 mL syringes (Fisher Scientific) containing 2 mL SD5X2.5 cell suspension were prepared. Prior to each inoculation, the piston of each syringe was removed and 2 mL SD5X2.5 cell suspension was added. The piston was then carefully reinserted and residual air was removed by pressing the plunger until a meniscus of liquid was visible at the tip of the syringe. The syringe was then sealed with a BD™ Combi™ Luer-Lock plug (Fisher Scientific). The inoculated syringes were incubated at 30° C. with 200 rpm shaking. Samples were taken for HPLC analysis at the start of the experiment (0 hours) and after 19.9 hours, 28.8 hours, 41.6 hours, 52 hours and 66.3 hours. At the designated time points, the cultivation broth was filtered through a 0.22 μm Millex-GP Med Syringe Filter Unit with a PES membrane (Merck Millipore) and collected in a 1.5 mL Eppendorf tube. To gain sufficient coverage of the fermentation kinetics, only 2 out of the 4 isolates from each strain were sampled at 28.8 hours, 41.6 hours, 52 hours and 66.3 hours (at 0 hours and at 19.9 hours all isolates were sampled). For HPLC analysis, the filtered culture broth was mixed in a 1:1 ratio with 5 mM H₂SO₄ and sent for HPLC analysis at the department for Analytical Development at Novozymes A/S, Denmark.

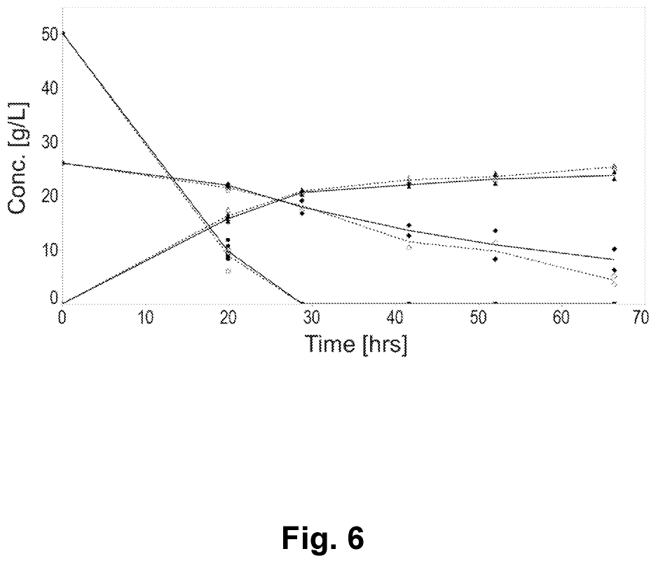

The results from the HPLC analysis are shown in Table 4 below and graphically in FIG. 6. Constitutive HXT2 expression improved xylose consumption in the SD5X2.5 fermentations as only 4.37 g/L xylose was left in the FYD1547 fermentations by the end of the experiment (66.3 hours) compared to 8.17 g/L for the FYD853 fermentations.

TABLE 4
Anaerobic syringe fermentations in SX2.5 media.

| Strain | Time [hours] | 0 | 19.9 | 28.8 | 41.6 | 52 | 66.3 |
|---|---|---|---|---|---|---|---|
| FYD853 | Glucose [g/L] | 50.13 | 10.70 | 0*** | 0*** | 0*** | 0*** |
| | Xylose [g/L] | 23.00 | 22.14 | 17.92 | 13.58 | 10.91 | 8.17 |
| | EtOH [g/L]* | 0** | 15.36 | 20.62 | 22.04 | 23.11 | 23.77 |
| | EtOH yield [g/g]* | — | — | — | — | — | 0.35 |
| | Final OD_{600 nm} | — | — | — | — | — | 5.79 |
| FYD1547 | Glucose [g/L] | 50.13 | 9.31 | 0*** | 0*** | 0*** | 0*** |
| | Xylose [g/L] | 23.00 | 21.51 | 18.18 | 11.47 | 9.75 | 4.37 |
| | EtOH [g/L]* | 0** | 16.19 | 20.87 | 22.96 | 23.55 | 25.34 |
| | EtOH yield [g/g]* | — | — | — | — | — | 0.35 |
| | Final OD_{600 nm} | — | — | — | — | — | 9.65 |

*EtOH yield is calculated as g EtOH per g xylose consumed.
**No EtOH was detected.
***No xylose was detected.

### Example 5: Construction of Yeast Strains McTs1084-1087

This example describes the construction of yeast strains McTs1084, McTs1085, McTs1086 and McTs1087 which express a xylose isomerase and contain one copy of the hxt2 gene under control of the TEF2 promoter integrated at the both XII-2 loci in the diploid strain.

Synthetic DNA containing the 50 bp 5′ flank to XII-2 locus, TEF2 promoter (SEQ ID NO: 50), hxt2 gene, TIP1 terminator (SEQ ID NO: 51) and 50 bp 3′ flank to XII-3 locus was ordered as a linear DNA String from ThermoFisher and designated 17AAPWNP (SEQ ID NO: 29). The synthetic DNA was amplified by PCR using primers 1222569 and 1222570. The PCR amplification reaction was performed using Phusion® Hot Start DNA Polymerase (Thermo Fisher) per the manufacturer's instructions. Each PCR was composed of 5 ng 17AAPWNP (SEQ ID NO: 29) synthetic linear DNA as template, 50 pmol primer 1225569, 50 pmol primer 1225570, 0.1 mM each dATP, dGTP, dCTP, dTTP, 1× Phusion HF Buffer, and 2 units Phusion Hot Start DNA polymerase in a final volume of 50 μL. The PCR was performed in a T100™ Thermal Cycler (Bio-Rad Laboratories, Inc.) programmed for one cycle at 98° C. for 3 minutes followed by 10 cycles each at 98° C. for 10 seconds, 50° C. for 20 seconds, and 72° C. for 2 minute followed by 25 each at 98° C. for 10 seconds, 58° C. for 20 seconds, and 72° C. for 2 minutes with a final extension at 72° C. for 5 minutes. Following thermocycling, the PCR reaction product of 2.7 kb was gel isolated and cleaned up using the NucleoSpin Gel and PCR clean-up kit (Machery-Nagel).

The yeast strain S. cerevisiae MBG4982 was transformed with the PCR amplified 17AAPWNP DNA. To aid homologous recombination of the hxt2 containing cassette into the XII-2 locus a plasmid containing Cas9 and guide RNA specific to XII_2 (pMIBa359) was also used in the transformation. The plasmid and PCR amplified 17AAPWNP DNA were transformed into S. cerevisiae strain MBG4982 using a yeast electroporation protocol. Transformants were selected on YPD+cloNAT to select for transformants that contain the CRISPR/Cas9 plasmid pMIBA359.

To ensure correct integration of the hxt2 expression cassette into the XII-2 locus MBG4982, PCR across the locus was performed. To generate genomic template DNA from transformants, a colony was resuspended in 10 μl sterile water then 40 μl Y-lysis buffer (Zymo Research) and 2 μl zymolyase (Zymo Research) was added. Samples were incubated at 37° C. for 30 minutes then 1 μl of the lysed cells was used in the following PCR reaction. The PCR amplification reaction was performed using Phusion® Hot Start DNA Polymerase (Thermo Fisher) per the manufacturer's instructions. Each PCR was composed of 1 μl zymolyase treated cells as DNA template, 50 pmol primer XII-2 external forward, 50 pmol primer XII-2 external reverse, 0.1 mM each dATP, dGTP, dCTP, dTTP, 1× Phusion HF Buffer, and 2 units Phusion Hot Start DNA polymerase in a final volume of 50 μL. The PCR was performed in a T100™ Thermal Cycler (Bio-Rad Laboratories, Inc.) programmed for one cycle at 98° C. for 3 minutes followed by 32 cycles each at 98° C. for 10 seconds, 54° C. for 20 seconds, and 72° C. for 2 minute with a final extension at 72° C. for 5 minutes. Following thermocycling, 5 μl from each PCR reaction was visualized on a 0.7% TBE agarose gel with ethidium bromide. Colony with the correct size PCR product of 3.8 kb were Sanger sequenced using primers 1220142 and 1222570. Four isolates that had the correct integration cassette by sequencing were selected and named McTs1084, McTs1085, McTs1086 and McTs1087.

### Example 6: Evaluation of Aerobic Growth of Genetically Engineered Strains Comprising a Heterologous Polynucleotide Expressing HXT2

To evaluate xylose utilization in aerobic growth, the strains from Examples 1 and 5 were streaked on fresh YPD agar plates and incubated at 30° C. for 2 days. Three mL YPD cultures were prepared for each strain then 150 μl of this inoculated YPD culture was added to 10-11 wells of a 96 Well Clear Flat Bottom Polystyrene Microplate (Corning). The plate was grown for 3 days at 32° C. with shaking at 300 rpm. A copy of the plate was made by inoculating 150 μl of fresh YPD in 96 Well Clear Flat Bottom Polystyrene Not Treated Microplate (Corning) with 4 μl from the previous plate. The new copy plate was incubated for 1 days at 32° C. with shaking at 300 rpm. This plate was used to inoculate 96 well plates containing 150 μl media with xylose (SX2), glucose (SD2) or xylose+glucose (SX1/SD1) as the sole carbon source. The media was dispensed into each plate using a Beckman Coulter robotic system. For each media three replicate plates were made in the same way. The plates were incubated at 32° C. with shaking at 300 rpm for 0 h, 21.5 hr, or 27.5 hr. At each timepoint, the growth of the wells was assessed by OD_{595 nm} in a Beckman Coulter DTX 880 Multimode Detector plate reader.

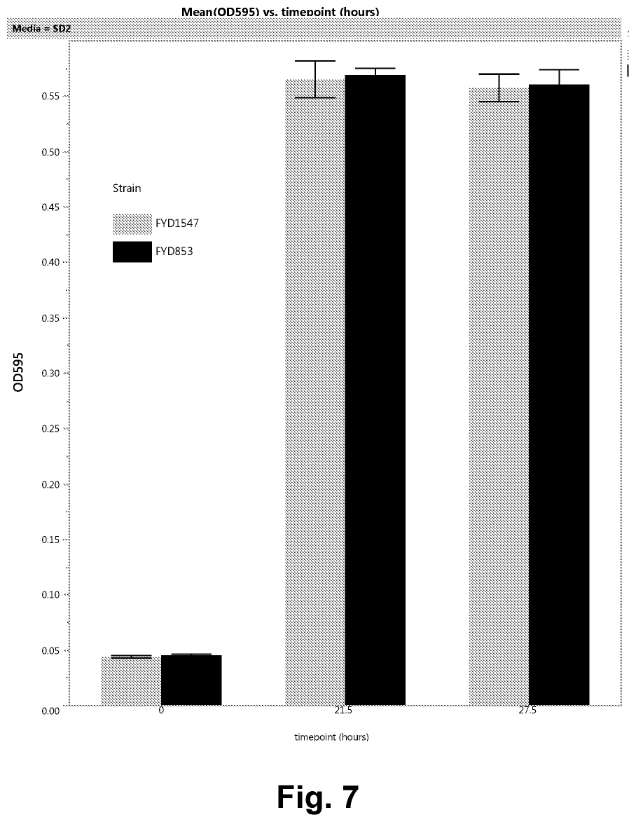

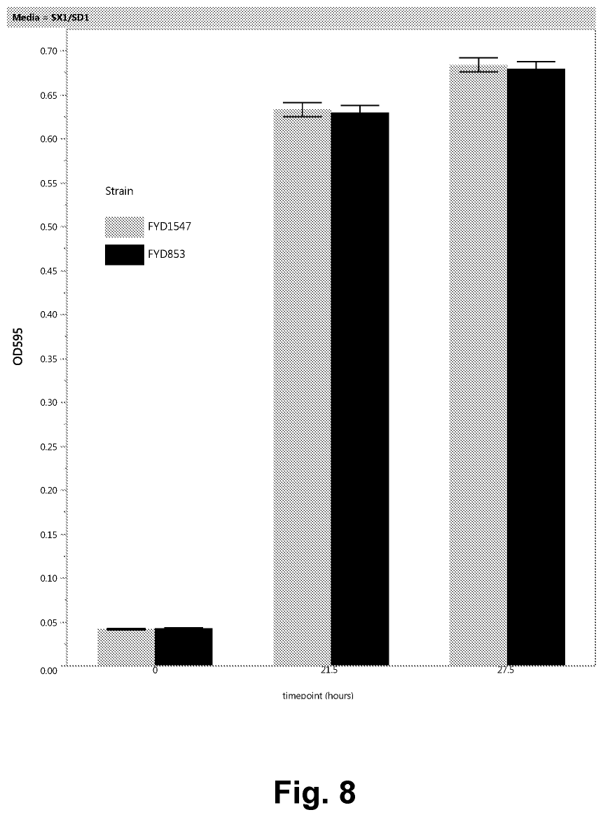

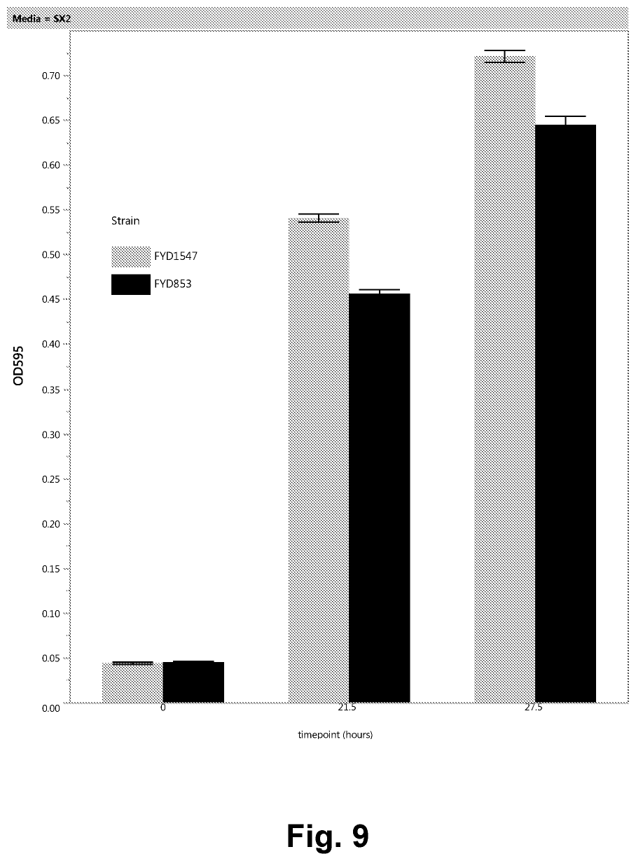

The average of replicate wells within the plate for yeast strains FYD853 and FYD1547 at each timepoint are show in FIGS. 7-9 (for SD2, SX1/SD1, and SX2 media, respectively). In SX2 media strain FYD1547 containing the heterologous hxt2 cassette has an increased growth over its parent strain FYD853 of 18% at 21.5 hours and 11% and 27.5 hours.

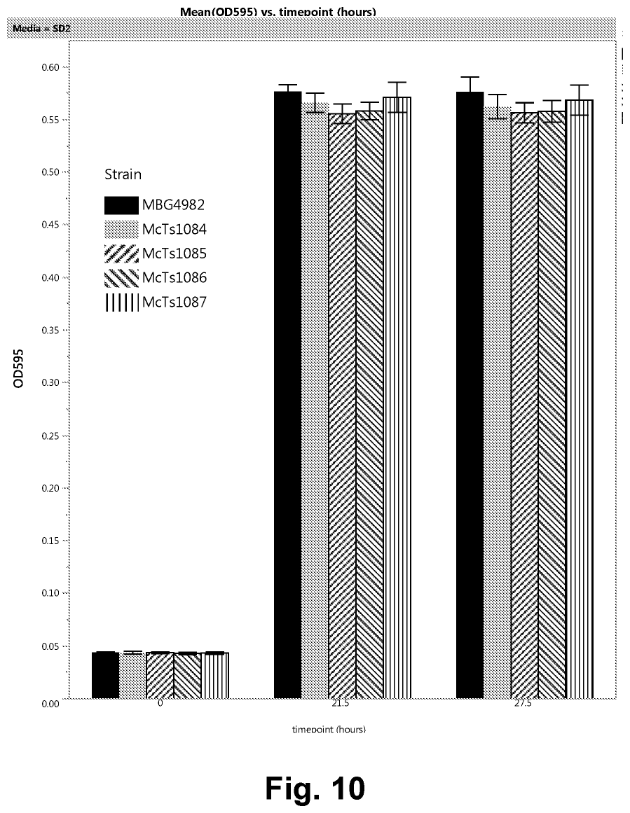

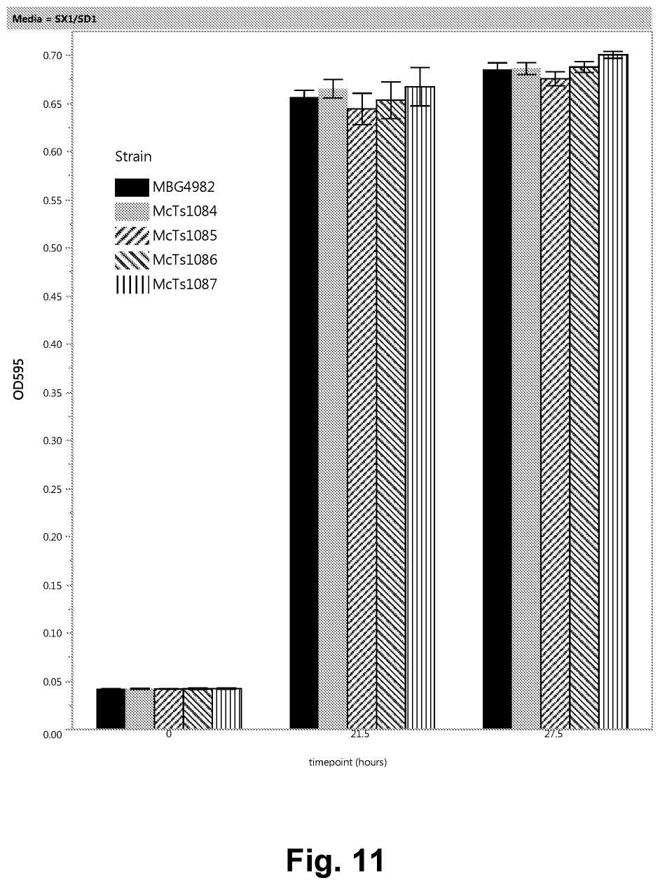

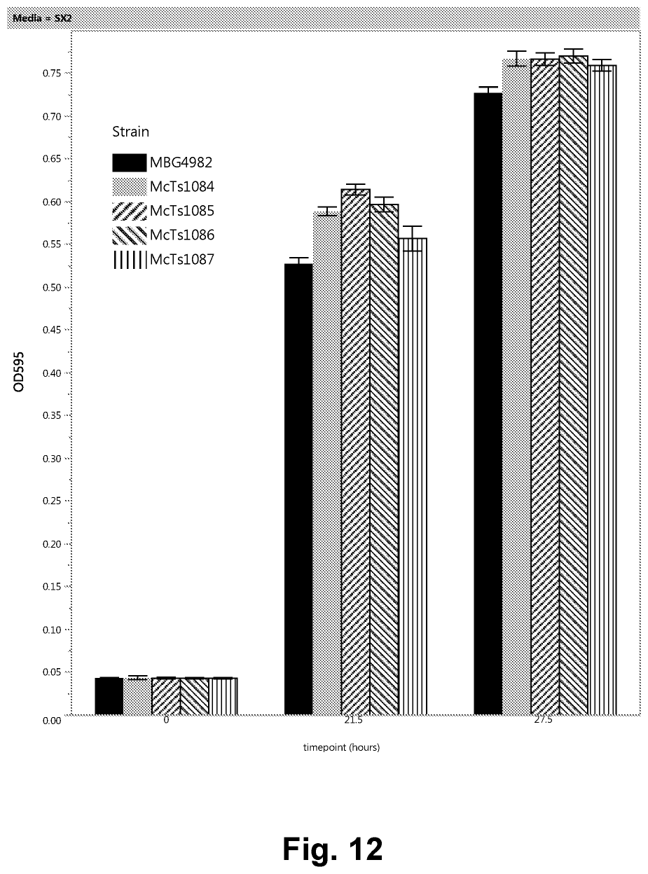

Results for strains McTs1084-1087 and MBG4982 are shown in FIGS. 10-12. The average range of improvement for the four isolates with the heterologous hxt2 cassette was 5-11% at 21.5 hours and 3.9-5.5% at 27.5 hours over parent strain MBG4982.

### Example 7: Fermentation of Genetically Engineered Strains Comprising a Heterologous Polynucleotide Expressing HXT2

To evaluate xylose utilization for ethanol production in anaerobic fermentations, strains were streaked on fresh YPD agar plates and incubated at 30° C. for 2 days. Three mL YPD cultures were prepared for each strain then 150 μl of this inoculated YPD culture was added to 10-11 wells of a 96 Well Clear Flat Bottom Polystyrene Microplate (Corning). The plate was grown for 3 days at 32° C. with shaking at 300 rpm. A copy of the plate was made by inoculating 150 μl of fresh YPD in 96 Well Clear Flat Bottom Polystyrene Not Treated Microplate (Corning) with 4 μl from the previous plate. The new copy plate was incubated for 1 days at 32° C. with shaking at 300 rpm. This plate was used to inoculate 96 deep well plates containing 500 μl media with 6% xylose (SX6), 6% dextrose (SD6) or xylose+dextrose (SX3/SD3) as the sole carbon source. The media was dispensed into each plate using a Beckman Coulter robotic system. The plates covered with a CO₂ release Sandwich cover (Enzyscreen), clamped, and incubated at 32° C. for 50 hr without shaking. Fermentation was stopped by the addition of 100 μL of 8% H₂SO₄, followed by centrifugation at 3000 rpm for 10 min. Ethanol and xylose in the supernatant was analyzed by HPLC.

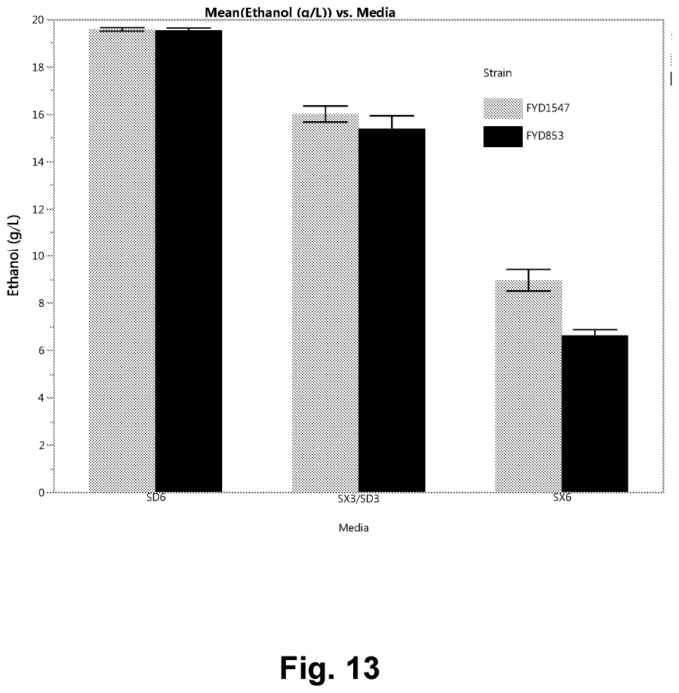

FIG. 13 shows the ethanol titers from the fermentations in SD6, SX6, SX3/SD3 media for strains FYD853 and FYD1547. The strain containing the heterologous hxt2 cassette, FYD1547, showed a 35% increased ethanol tier in the 6% xylose media (SX6 media) compared to parent strain FYD853 which lacks the heterologous hxt2 cassette.

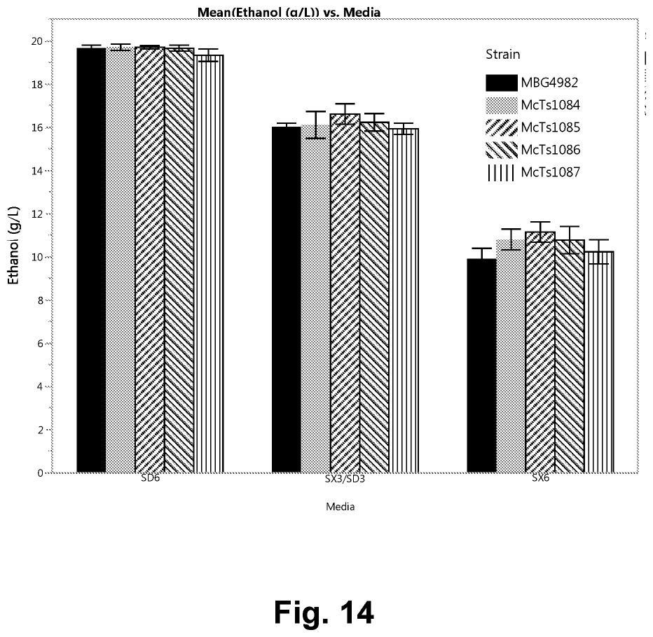

FIG. 14 shows the ethanol titers from the fermentations in SD6, SX6, SX3/SD3 media for stains McTs1084-1087 and MBG4982. The strains containing the heterologous hxt2 cassette showed a 3-12% ethanol titer increase compared to parent strain MBG4982.

Table 5 shows the increase in xylose consumption for strains containing the heterologous hxt2 cassette above their parent strain without containing the heterologous hxt2 cassette. Strain FYD1547 had an increase xylose consumption of 18.4% and stains McTs1084-1087 ranged from 4.1%-12.7% above the parent strain depending on the isolate.

TABLE 5
Xylose consumption in anaerobic fermentations.

| | | | | Xylose |
|---|---|---|---|---|
| | | | | consumption |
| | Mean Xylose | Standard | | above parent |
| Strain | (g/L) | Deviation | N | strain |
| FYD853 | 31.427 | 0.573 | 10 | N/A |
| FYD1547 | 25.642 | 1.1114 | 10 | 18.4% |
| MBG4982 | 23.383 | 1.2093 | 13 | N/A |
| McTs1084 | 21.211 | 1.2494 | 11 | 9.3% |
| McTs1085 | 20.422 | 1.2279 | 11 | 12.7% |
| McTs1086 | 21.297 | 1.6098 | 11 | 8.9% |
| McTs1087 | 22.424 | 1.4327 | 11 | 4.1% |
| P51-F11 | 46.648 | 0.0404 | 14 | N/A |
| McTs1100 | 46.636 | 0.0688 | 14 | 0.0% |
| McTs1101 | 46.644 | 0.0458 | 14 | 0.0% |
| McTs1102 | 46.653 | 0.0401 | 14 | 0.0% |
| P52-B02 | 46.926 | 0.3678 | 16 | N/A |
| McTs1103 | 46.537 | 0.0562 | 14 | 0.8% |
| McTs1104 | 47.489 | 0.0943 | 14 | −1.2% |
| McTs1105 | 47.689 | 0.1243 | 14 | −1.6% |
| P55-H01 | 48.155 | 0.2256 | 12 | N/A |
| McTs1106 | 48.126 | 0.0861 | 14 | 0.1% |
| McTs1107 | 48.259 | 0.0957 | 14 | −0.2% |
| McTs1108 | 48.224 | 0.1037 | 14 | −0.1% |

### Example 8: Construction of Yeast Strains Expressing XR/XDH Xylose Utilization Pathway

This example describes the construction of yeast strains P51-F11, P52-B02, P55-H01 which lack a xylose isomerase but contain the D-xylose reductase/xylitol dehydrogenase (XR/XDH) xylose utilization pathway at both X-3 loci in the diploid strain Ethanol Red.

A xylose utilization pathway containing a D-xylose reductase (XR), xylitol dehydrogenase (XDH), xylulokinase (XK), transaldolase (TAL), and phosphoglucomutase (PGM2 from Saccharomyces cerevisiae) was integrated into both X-3 loci of the diploid strain Ethanol Red. The promoters used to express each gene were: TDH3 promoter for xylitol dehydrogenase, ADH1 promoter for xylulokinase, PGK1 for D-xylose reductase, RPL18B for transaldolase, and TEF2 (SEQ ID NO: 50), for phosphoglucomutase. Three strains were designated P51-F11, P52-B02, and P55-H01 which contain different XR, XDH, XK, and or TAL genes. Strain P55-H01 contained the following genes at both X-3 loci in the diploid strain Ethanol Red: Saccharomyces cerevisiae TAL (encoding SEQ ID NO: 40), Spathaspora girioi XDH (encoding SEQ ID NO: 43), Pseudomonas fluorescens XK (encoding SEQ ID NO: 45), and Aspergillus nigerXR (encoding SEQ ID NO: 47). Strain P51-F11 contains the following genes at both X-3 loci in the diploid strain Ethanol Red: Candida glabrate TAL (encoding SEQ ID NO: 41), Spathaspora girioi XDH (encoding SEQ ID NO: 43), Scheffersomyces stipitis XK (encoding SEQ ID NO: 46), Aspergillus oryzae XR (encoding SEQ ID NO: 48). Strain P52-B02 contains the following genes at both X-3 loci in the diploid strain Ethanol Red: Saccharomyces dairenensis TAL (encoding SEQ ID NO: 42), Candida tenuis XDH (encoding SEQ ID NO: 44), Scheffersomyces stipitis XK (encoding SEQ ID NO: 46), Aspergillus niger XR (encoding SEQ ID NO: 47). All strains also have the phosphoglucomutatase gene from Saccharomyces cerevisiae (encoding SEQ ID NO: 49) at both X-3 loci in the diploid strain Ethanol Red.

Strains containing the five-gene pathway, XR, XDH, XK, TAL and PGM2, were made using synthetic DNA encoding each promoter, gene and terminator. The synthetic DNA containing the promoter and terminator fragments were ordered as cloned DNA from GeneArt in plasmids and are as indicated in below table. 16ACZJXP (500 bp 5′ flank for X-3 site and TDH3 promoter), 16ACT3QP (PDC6 terminator and ADH1 promoter), 16ACZJWP (TEF1 terminator and PGK1 promoter), and 16ACZJVP (ADH3 terminator and RPL18B promoter). The fragment that contains the PGM2 gene was also ordered as cloned DNA and named 16ACZJYP. This plasmid contained the PRM9 terminator, TEF2 promoter, PGM2 gene, ENO2 terminator and 300 bp 3′ X-3 flanking DNA. The linear fragments for transformation were generated by PCR with oligos indicated in Table 6.

TABLE 6
PCR oligos used for amplification of fragments used
in yeast strains expressing XR/XDH xylose pathway.

| GeneArt | | |
|---|---|---|
| plasmid | 5′ PCR oligo | 3′ PCR oligo |
| 16ACZJXP | 1221575 | 1221470 |
| 16ACT3QP | 1221475 | 1221746 |
| 16ACZJWP | 1221471 | 1221754 |
| 16ACZJVP | 1221756 | 1221472 |
| 16ACZJYP | 1221473 | 1221747 |

In addition to the above five linear DNA fragment generated by PCR from synthetic DNA plasmids, four additional DNAs were used in the transformation to integrate the five-gene pathway at both X-3 loci in Ethanol Red. For each transformation one TAL, XDH, XK, and XR fragment was used in combination with the above five linker pieces. The fragments had homology on the 5′ and 3′ ends to their adjoining fragment. The CRISPR Cas9 plasmid pMcTs442 containing a gRNA to X-3 site and Cas9 was used to aid homology recombination of the nine DNA fragments into both X-3 loci in diploid Ethanol Red using a yeast electroporation protocol. Transformants were selected on YPD+cloNAT to select for transformants that contain the CRISPR/Cas9 plasmid pMcTs442. Transformants were screened for integration of the pathway by PCR and confirmed by sequencing. Tables 7 and 8 shows details of the pathway genes and corresponding strains.

TABLE 7
Linear DNA strings encoding genes used to generated
yeast strains expressing XR/XDH xylose pathway.

| GeneArt | | | Enzyme | Coding source | |
|---|---|---|---|---|---|
| String name | promoter | Gene ID | type | Organism | terminator |
| 16ACZYEP | RPL18B | P43YZ8 | TAL | Saccharomyces | PRM9 |
| | | (SEQ ID NO: 40) | | cerevisiae | |
| 16ACZYGP | RPL18B | Q6FXG5 | TAL | Candida glabrata | PRM9 |
| | | (SEQ ID NO: 41) | | | |
| 16ACZYHP | RPL18B | G0WH02 | TAL | Saccharomyces | PRM9 |
| | | (SEQ ID NO: 42) | | dairenensis | |
| 16ACZYVP | TDH3 | A0A173DUJ8 | XDH | Spathaspora girioi | PDC6 |
| | | (SEQ ID NO: 43) | | | |
| 16ACZYXP | TDH3 | G3B9C7 | XDH | Candida tenuis | PDC6 |
| | | (SEQ ID NO: 44) | | | |
| 16ACZYRP | ADH1 | Q4KC52 | XK | Pseudomonas | TEF1 |
| | | (SEQ ID NO: 45) | | fluorescens | |
| 16ACZYSP | ADH1 | A3GF74 | XK | Scheffersomyces | TEF1 |
| | | (SEQ ID NO: 46) | | stipitis | |
| 16ACZYNP | PGK1 | A2Q8B5 | XR | Aspergillus niger | ADH3 |
| | | (SEQ ID NO: 47) | | | |
| 16ACZYMP | PGK1 | EFP2C7GS0 | XR | Aspergillus | ADH3 |
| | | (SEQ ID NO: 48) | | oryzae | |

TABLE 8
XR/XDH pathway yeast strains.

| Strain | | | | |
|---|---|---|---|---|
| name | TAL | XDH | XK | XR |
| P55-H01 | P43YZ8 | A0A173DUJ8 | Q4KC52 | A2Q8B5 |
| | (SEQ ID | (SEQ ID | (SEQ ID | SEQ ID |
| | NO: 40) | NO: 43) | NO: 45) | NO: 47) |
| P51-F11 | Q6FXG5 | A0A173DUJ8 | A3GF74 | EFP2C7GS0 |
| | (SEQ ID | (SEQ ID | (SEQ ID | (SEQ ID |
| | NO: 41) | NO: 43) | NO: 46) | NO: 48) |
| P52-B02 | G0WH02 | G3B9C7 | A3GF74 | A2Q8B5 |
| | (SEQ ID | (SEQ ID | (SEQ ID | SEQ ID |
| | NO: 42) | NO: 44) | NO: 46) | NO: 47) |

### Example 9: Construction of Yeast Strains Expressing XR/XDH Xylose Utilization Pathway and Comprising a Heterologous Polynucleotide Expressing HXT2

This example describes is the construction of the yeast strains McTs1100, McTs1101, McTs1102, McTs1103, McTs1104, McTs1105, McTs1106, McTs1107, McTs1108 which contain a heterologous polynucleotide expressing the hxt2 gene under control of the TEF2 promoter integrated at the both XII-2 loci in XR/XDH xylose pathway strains P51-F11, P52-B02 and P55-H01.

The yeast strains P51-F11, P52-B02, and P55-H01 MBG4982 were transformed with the PCR amplified 17AAPWNP DNA (supra). To aid homologous recombination of the hxt2 containing cassette into the XII-2 locus a plasmid containing Cas9 and guide RNA specific to XII-2 (pMIBa359) was also used in the transformation. The plasmid and PCR amplified 17AAPWNP DNA were transformed into S. cerevisiae strains P51-F11, P52-B02, and P55-H01 using a yeast electroporation protocol. Transformants were selected on YPD+cloNAT to select for transformants that contain the CRISPR/Cas9 plasmid pMIBa359.

To ensure correct integration of the heterologous hxt2 expression cassette into the XII-2 locus MBG4982, PCR across the locus was performed. To generate genomic template DNA from transformants, a colony was resuspended in 10 μl sterile water then 40 μl Y-lysis buffer (Zymo Research) and 2 μl zymolyase (Zymo Research) was added. Samples were incubated at 37° C. for 30 minutes then 1 μl of the lysed cells was used in the following PCR reaction. The PCR amplification reaction was performed using Phusion® Hot Start DNA Polymerase (Thermo Fisher) per the manufacturer's instructions. Each PCR was composed of 1 μl zymolyase treated cells as DNA template, 50 pmol primer XII-2 external forward, 50 pmol primer XII-2 external reverse, 0.1 mM each dATP, dGTP, dCTP, dTTP, 1× Phusion HF Buffer, and 2 units Phusion Hot Start DNA polymerase in a final volume of 50 μL. The PCR was performed in a T100™ Thermal Cycler (Bio-Rad Laboratories, Inc.) programmed for one cycle at 98° C. for 3 minutes followed by 32 cycles each at 98° C. for 10 seconds, 54° C. for 20 seconds, and 72° C. for 2 minute with a final extension at 72° C. for 5 minutes. Following thermocycling, 5 μl from each PCR reaction was visualized on a 0.7% TBE agarose gel with ethidium bromide. Colony with the correct size PCR product of 3.8 kb were Sanger sequenced using primers 1220142 and 1222570. Three isolates that had the correct integration cassette by sequencing for each of the three strain backgrounds were selected. The three isolates from strain P51-F11 with the hxt2 cassette were named McTs1100, McTs1101, McTs1102. The three isolates from P52-B02 with the hxt2 cassette were name McTs1103, McTs1104, McTs1105. The three isolates from strain background P55-H01 with the hxt2 cassette were named McTs1106, McTs1107, McTs1108.

### Example 10: Evaluation of Aerobic Growth of Genetically Engineered Strains Expressing XR/XDH Xylose Utilization Pathway and Comprising a Heterologous Polynucleotide Expressing HXT2

To evaluate xylose utilization in aerobic growth, the strains from Example 9 were streaked on fresh YPD agar plates and incubated at 30° C. for 2 days. Three-mL YPD cultures were prepared for each strain then 150 μl of this inoculated YPD culture was added to 10-11 wells of a 96 Well Clear Flat Bottom Polystyrene Microplate (Corning). The plate was grown for 3 days at 32° C. with shaking at 300 rpm. A copy of the plate was made by inoculating 150 μl of fresh YPD in 96 Well Clear Flat Bottom Polystyrene Not Treated Microplate (Corning) with 4 μl from the previous plate. The new copy plate was incubated for 1 days at 32° C. with shaking at 300 rpm. This plate was used to inoculate 96-well plates containing 150 μl media with 2% xylose (SX2), 2% dextrose (SD2) or 1% xylose+1% glucose (SX1/SD1) as the sole carbon source. The media was dispensed into each plate using a Beckman Coulter robotic system. For each media five replicate plates were made in the same way. The plates were incubated at 32° C. with shaking at 300 rpm for 0 h, 21.5 hr, or 27.5 hr, 45 hr, or 52 hr. At each timepoint, the growth of the wells was assessed by OD_{595 nm} in a Beckman Coulter DTX 880 Multimode Detector plate reader.

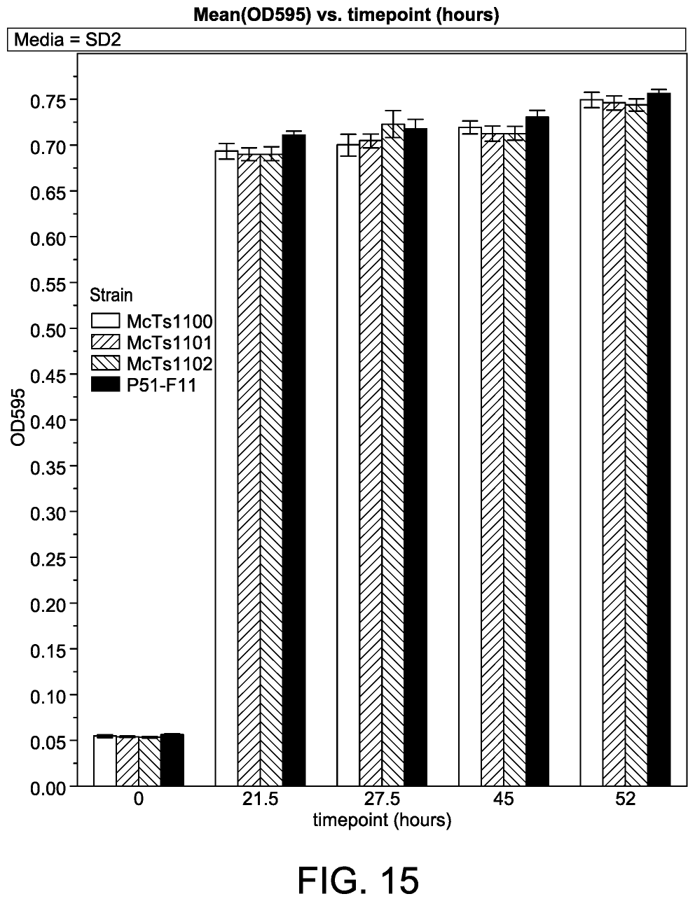

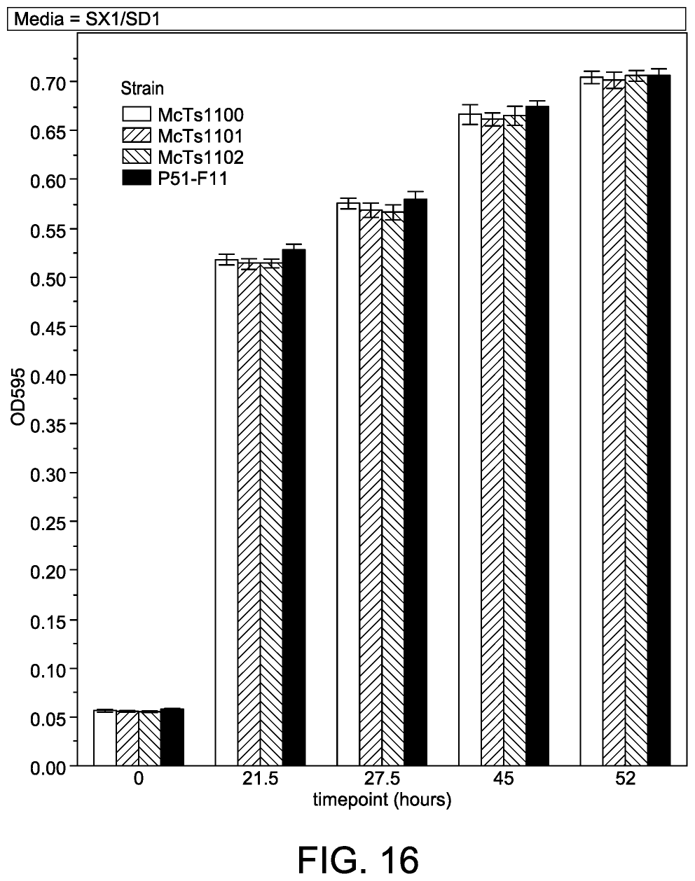

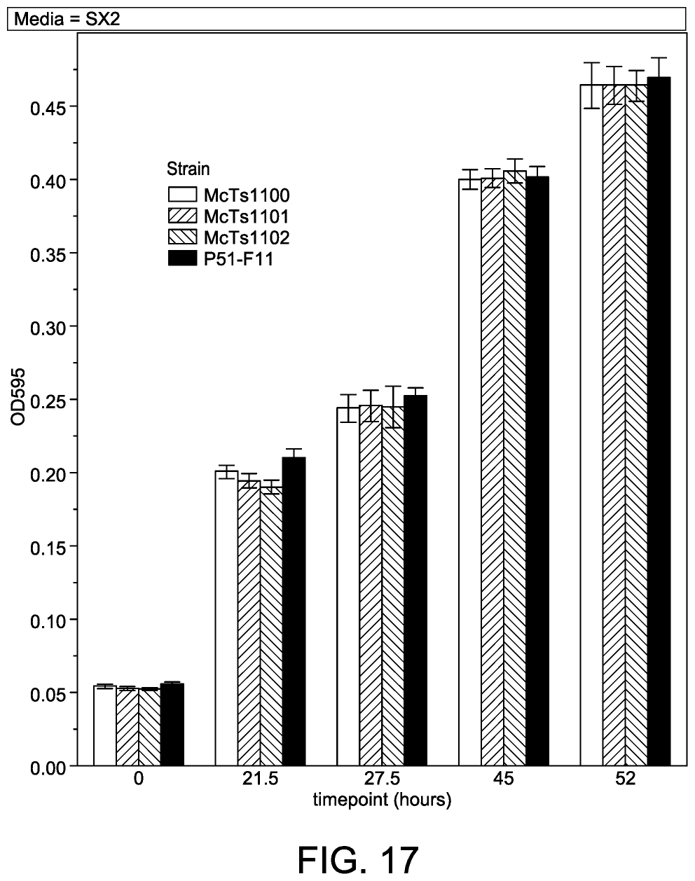

The average of replicate wells within the plate at each timepoint and each of the three medias are show in FIGS. 15-17 for strain background P51-F11. Unlike the results above for strains comprising a heterologous polynucleotide expressing HXT2 with a xylose isomerase (XI), there was no improvement in growth for strains comprising the heterologous polynucleotide expressing HXT2 with the XR/XDH xylose utilization pathway (McTs1100, McTs1101, McTs1102) compared to parent P51-F11 in SD2, SX1/SD1 or SX2 media at any of the timepoints.

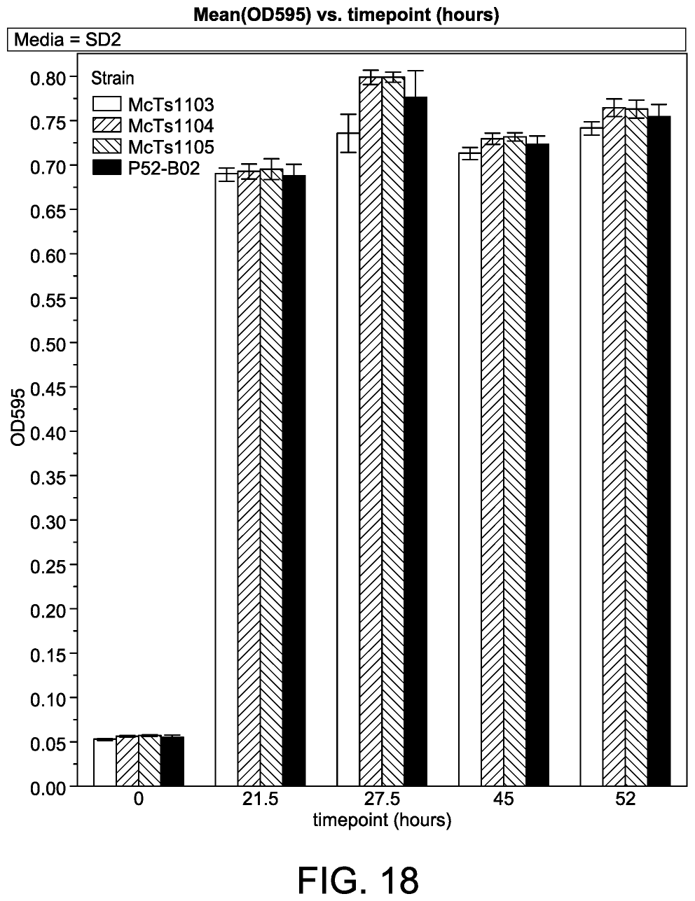

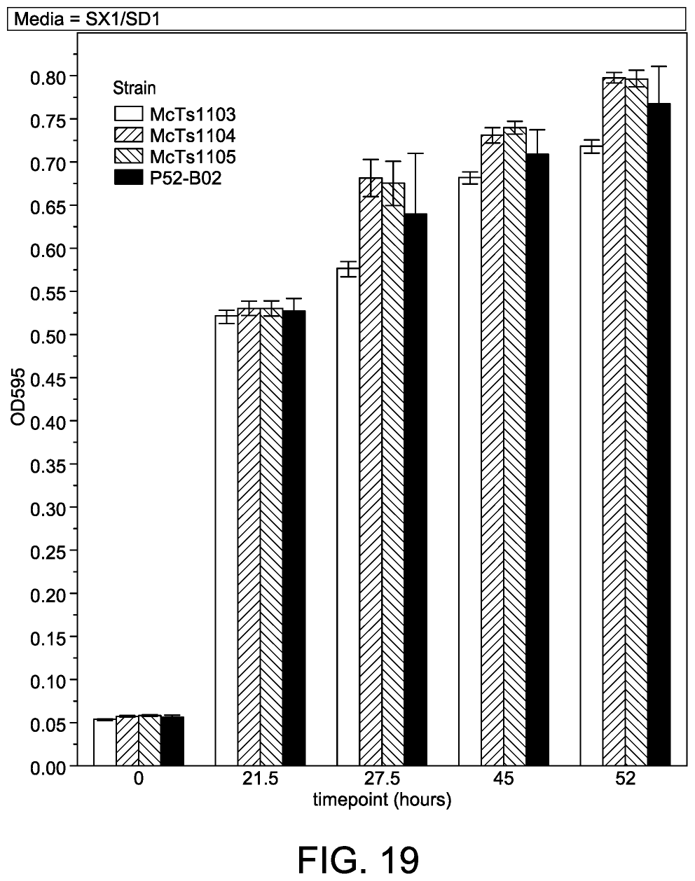

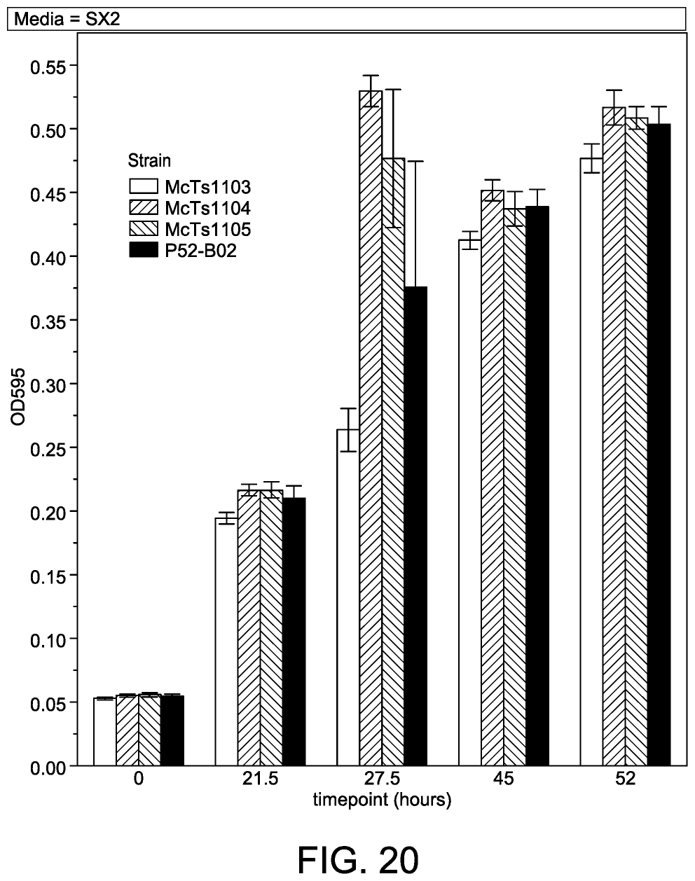

Similarly, FIGS. 18-20 show no benefit in growth for strains comprising a heterologous polynucleotide expressing HXT2 when expressed with the XR/XDH xylose utilization pathway (McTs1103, McTs1104, McTs1105) compared to parent P52-B02.

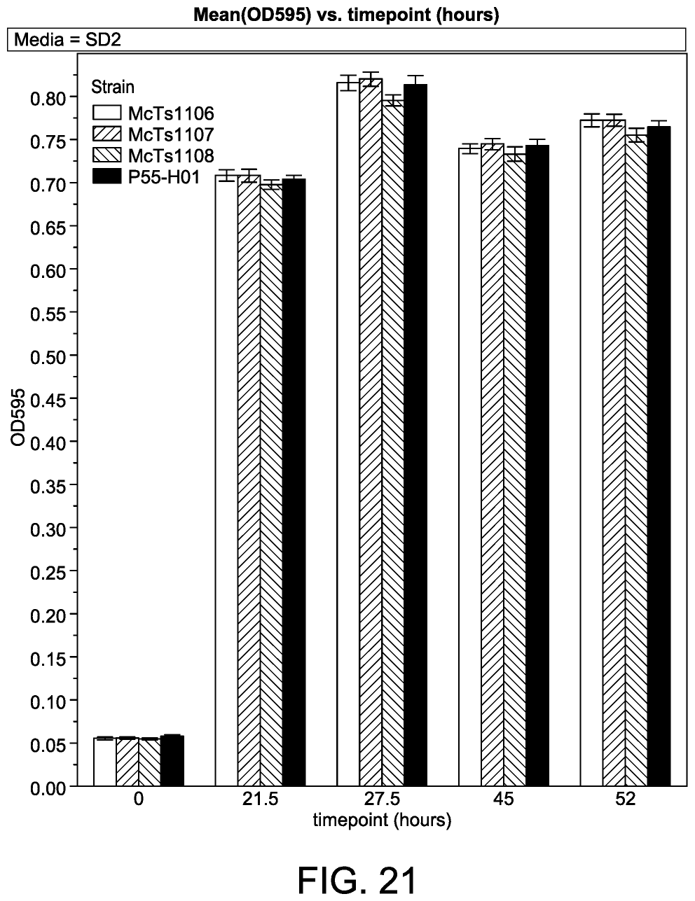

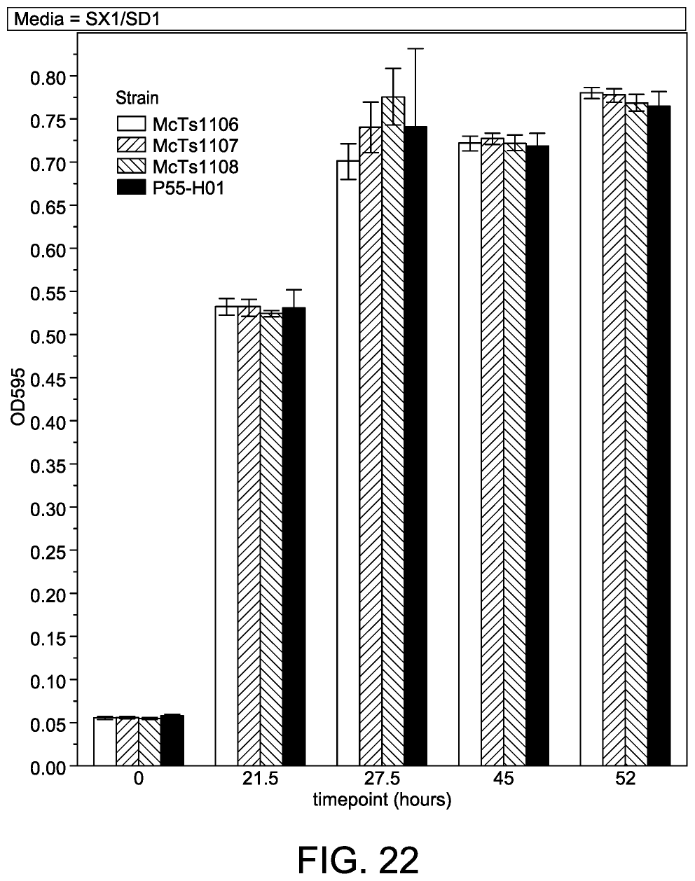

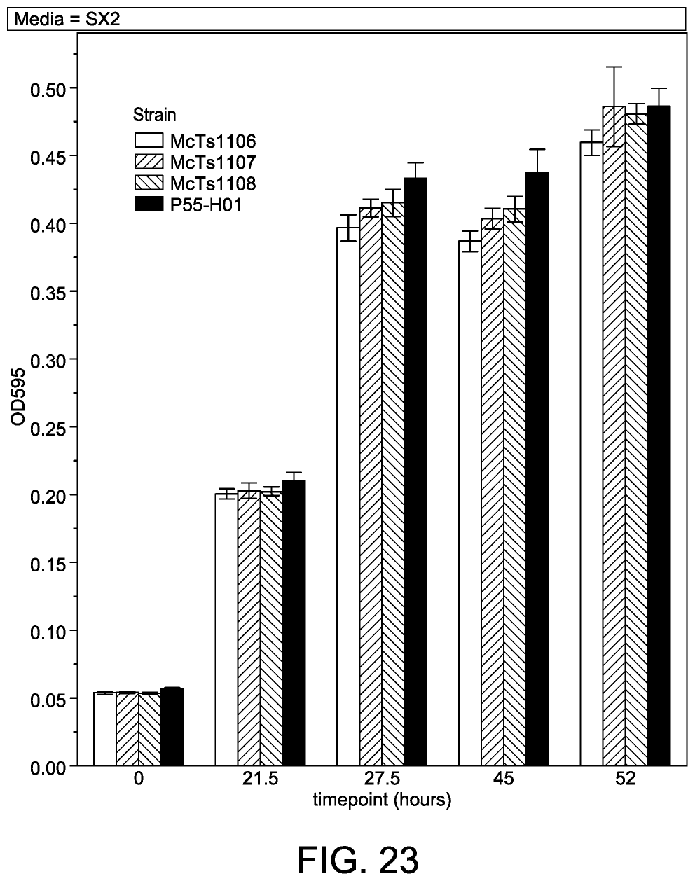

Likewise, FIGS. 21-23 show no benefit in growth for strains comprising a heterologous polynucleotide expressing HXT2 when expressed with the XR/XDH xylose utilization pathway (McTs1106, McTs1107, McTs1108) compared to parent P55-H01.

Additionally, as shown in Table 5, there was no increase in xylose consumption in SX2 media for strains comprising a heterologous polynucleotide expressing HXT2 when expressed with the XR/XDH xylose utilization pathway compared to parent strains lacking the heterologous polynucleotide.

Although the foregoing has been described in some detail by way of illustration and example for the purposes of clarity of understanding, it is apparent to those skilled in the art that any equivalent aspect or modification may be practiced. Therefore, the description and examples should not be construed as limiting the scope of the invention.

1. A recombinant yeast cell comprising a heterologous polynucleotide encoding a hexose transporter and a heterologous polynucleotide encoding a xylose isomerase, wherein the hexose transporter has at least 70% sequence identity to SEQ ID NO: 2; and wherein the yeast cell is capable of fermenting xylose.

2. The recombinant cell of claim 1, wherein the heterologous polynucleotide encodes a hexose transporter that differs by no more than ten amino acids from SEQ ID NO: 2.

3. The recombinant cell of claim 1, wherein the heterologous polynucleotide encodes a hexose transporter having an amino acid sequence comprising or consisting of the amino acid sequence of SEQ ID NO: 2.

4. The recombinant cell of claim 1, wherein the heterologous polynucleotide encoding a hexose transporter comprises a coding sequence having at least 70% sequence identity to SEQ ID NO: 1.

5. The recombinant cell of claim 4, wherein the heterologous polynucleotide encoding a hexose transporter has a coding sequence that consists of SEQ ID NO: 1.

6. The recombinant cell of claim 1, wherein the heterologous polynucleotide encoding a hexose transporter comprises a coding sequence that hybridizes under at least medium stringency conditions with the full-length complementary strand of SEQ ID NO: 1.

7. The recombinant cell of claim 1, wherein the strain has a higher anaerobic growth rate on xylose compared to the same cell without the heterologous polynucleotide encoding a hexose transporter at about or after 4 days of incubation.

8. The recombinant cell of claim 1, wherein the strain has a higher xylose consumption compared to the same cell without the heterologous polynucleotide encoding a hexose transporter at about or after 40 hours fermentation.

9. The recombinant cell of claim 1, wherein the strain has a higher ethanol production compared to the same cell without the heterologous polynucleotide encoding a hexose transporter at about or after 40 hours fermentation.

10. The recombinant cell of claim 1, which is a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cell.

11. The recombinant cell of claim 1, which is a Saccharomyces cerevisiae cell.

12. The recombinant cell of claim 1, wherein the cell is capable of consuming more than 65% xylose in the medium at about or after 66 hours fermentation.

13. The recombinant cell of claim 1, wherein the cell is capable of consuming more than 65% glucose in the medium at about or after 66 hours fermentation.

14. The recombinant cell of claim 1, wherein the cell is capable of consuming more than 65% xylose in the medium, and is capable of consuming more than 65% glucose in the medium, at about or after 66 hours fermentation.

15. A process for producing ethanol, comprising:
a) cultivating the recombinant cell of claim 1 in a fermentable medium under suitable conditions to produce ethanol; and
b) recovering the fermentation product from the fermentation.

16. The process of claim 15, wherein cultivation is conducted under low oxygen (e.g., anaerobic) conditions.

17. The process of claim 15, wherein cultivation is conducted under aerobic conditions.

18. The process of claim 15, wherein an increased amount of glucose and xylose is consumed when compared to the process using an identical cell without the heterologous polynucleotide encoding a hexose transporter under the same conditions.

19. The process of claim 15, wherein more than 65% xylose in the medium is consumed, and more than 65% glucose in the medium is consumed, at about or after 66 hours fermentation.

20. The process of claim 15, wherein the process results in higher ethanol yield when compared to the process using an identical cell without the heterologous polynucleotide encoding a hexose transporter under the same conditions.


SEQUENCE LISTING (51 sequences)

SEQ ID NO: 1 - DNA, 1626 bp, Saccharomyces cerevisiae
atgtctgaattcgctactagcggcgttgaaagtggctctcaacaaacttctatccactctactccgatagtgcagaaattagagacggatgaatctcctattcaaaccaaatctgaatacactaacgctgaactcccagcaaagccaatcgccgcatattggactgttatctgtttatgtctaatgattgcatttggtgggtttgtctttggttgggatactggtaccatctctggttttgttaatcaaaccgatttcaaaagaagatttggtcaaatgaaatctgatggtacctattatctttcggacgtccggactggtttgatcgttggtatcttcaatattggttgtgccattggtgggttaaccttaggacgtctgggtgatatgtatggacgtagaattggtttgatgtgcgtcgttctggtatacatcgttggtattgtgattcaaattgcttctagtgacaaatggtaccagtatttcattggtagaattatctctggtatgggtgtcggtggtattgctgtcctatctccaactttgatttccgaaacagcaccaaaacacattagaggtacctgtgtttctttctatcagttaatgatcactctaggtattttcttaggttactgtaccaactatggtactaaagactactccaattcagttcaatggagagtgcctttgggtttgaactttgccttcgctattttcatgatcgctggtatgctaatggttccagaatctccaagattcttagtcgaaaaaggcagatacgaagacgctaaacgttctttggcaaaatctaacaaagtcaccattgaagatccaagtattgttgctgaaatggatacaattatggccaacgttgaaactgaaagattagccggtaacgcttcttggggtgagttattctccaacaaaggtgctattttacctcgtgtgattatgggtattatgattcaatccttacaacaattaactggtaacaattacttcttctattatggtactactattttcaacgccgtcggtatgaaagattctttccaaacttccatcgttttaggtatagtcaacttcgcatccactttcgtggccctatacactgttgataaatttggtcgtcgtaagtgtctattgggcggttctgcttccatggccatttgttttgttatcttctctactgtcggtgtcacaagcttatatccaaatggtaaagatcaaccatcttccaaggctgccggtaacgtcatgattgtctttacctgtttattcattttcttcttcgctattagttgggccccaattgcctacgttattgttgccgaatcttatcctttgcgtgtcaaaaatcgtgctatggctattgctgttggtgccaactggatttggggtttcttgattggtttcttcactcccttcattacaagtgcaattggattttcatacgggtatgtcttcatgggctgtttggtattttcattcttctacgtgtttttctttgtctgtgaaaccaagggcttaacattagaggaagttaatgaaatgtatgttgaaggtgtcaaaccatggaaatctggtagctggatctcaaaagaaaaaagagtttccgaggaataa

SEQ ID NO: 2 - protein, 541 aa, Saccharomyces cerevisiae
MSEFATSGVESGSQQTSIHSTPIVQKLETDESPIQTKSEYTNAELPAKPIAAYWTVICLCLMIAFGGFVFGWDTGTISGFVNQTDFKRRFGQMKSDGTYYLSDVRTGLIVGIFNIGCAIGGLTLGRLGDMYGRRIGLMCVVLVYIVGIVIQIASSDKWYQYFIGRIISGMGVGGIAVLSPTLISETAPKHIRGTCVSFYQLMITLGIFLGYCTNYGTKDYSNSVQWRVPLGLNFAFAIFMIAGMLMVPESPRFLVEKGRYEDAKRSLAKSNKVTIEDPSIVAEMDTIMANVETERLAGNASWGELFSNKGAILPRVIMGIMIQSLQQLTGNNYFFYYGTTIFNAVGMKDSFQTSIVLGIVNFASTFVALYTVDKFGRRKCLLGGSASMAICFVIFSTVGVTSLYPNGKDQPSSKAAGNVMIVFTCLFIFFFAISWAPIAYVIVAESYPLRVKNRAMAIAVGANWIWGFLIGFFTPFITSAIGFSYGYVFMGCLVFSFFYVFFFVCETKGLTLEEVNEMYVEGVKPWKSGSWISKEKRVSEE

SEQ ID NO: 3 - DNA, 24 bp, Artificial Sequence
  Artificial DNA Primer
ggttgtttatgttcggatgtgatg

SEQ ID NO: 4 - DNA, 51 bp, Artificial Sequence
  Artificial DNA Primer
acattatacgaagttatttaattaacatataatacatatcacataggaagc

SEQ ID NO: 5 - DNA, 43 bp, Artificial Sequence
  Artificial DNA Primer
attcagacattttgtaattaaaacttagattagattgctatgc

SEQ ID NO: 6 - DNA, 32 bp, Artificial Sequence
  Artificial DNA Primer
taattacaaaatgtctgaattcgctactagcg

SEQ ID NO: 7 - DNA, 41 bp, Artificial Sequence
  Artificial DNA Primer
aggttcccttttattcctcggaaactcttttttcttttgag

SEQ ID NO: 8 - DNA, 37 bp, Artificial Sequence
  Artificial DNA Primer
cgaggaataaaagggaaccttttacaacaaatatttg

SEQ ID NO: 9 - DNA, 43 bp, Artificial Sequence
  Artificial DNA Primer
cgtcaaggccgcatgcggccgcggaatagtgacgttgtgatgc

SEQ ID NO: 10 - DNA, 44 bp, Artificial Sequence
  Artificial DNA Primer
tgctatacgaagttatgtttaaacctaaactaacatcgcgatgc

SEQ ID NO: 11 - DNA, 41 bp, Artificial Sequence
  Artificial DNA Primer
aatatgggcgcgatcgctaagtacagacggaaactcacacc

SEQ ID NO: 12 - DNA, 42 bp, Artificial Sequence
  Artificial DNA Primer
cccatgaggcccagggcgcgccctacagatgttgctgcaacc

SEQ ID NO: 13 - DNA, 32 bp, Artificial Sequence
  Artificial DNA Primer
ttagcgatcgcgcccatatttagctcgtttgg

SEQ ID NO: 14 - DNA, 19 bp, Artificial Sequence
  Artificial DNA Primer
gctggctactcgttgctcg

SEQ ID NO: 15 - DNA, 44 bp, Artificial Sequence
  Artificial DNA Primer
tgctatacgaagttatgtttaaacctaaactaacatcgcattgc

SEQ ID NO: 16 - DNA, 22 bp, Artificial Sequence
  Artificial DNA Primer
tgatctgcagtagaatcagtgg

SEQ ID NO: 17 - DNA, 19 bp, Artificial Sequence
  Artificial DNA Primer
acttgtgtggatgccaacg

SEQ ID NO: 18 - protein, 439 aa, Bos taurus
MAKEYFPFTGKIPFEGKDSKNVMAFHYYEPEKVVMGKKMKDWLKFAMAWWHTLGGASADQFGGQTRSYEWDKAECPVQRAKDKMDAGFEIMDKLGIEYFCFHDVDLVEEAPTIAEYEERMKAITDYAQEKMKQFPNIKLLWGTANVFGNKRYANGASTNPDFDVVARAIVQIKNSIDATIKLGGTNYVFWGGREGYMSLLNTDQKREKEHMATMLGMARDYARAKGFKGTFLIEPKPMEPSKHQYDVDTETVIGFLKAHGLDKDFKVNIEVNHATLAGHTFEHELACAVDAGMLGSIDANRGDAQNGWDTDQFPIDNFELTQAMLEIIRNGGLGNGGTNFDAKIRRNSTDLEDLFIAHISGMDAMARALMNAADILENSELPAMKKARYASFDSGIGKDFEDGKLTFEQVYEYGKKVEEPKQTSGKQEKYETIVALHCK

SEQ ID NO: 19 - DNA, 4945 bp, Escherichia coli
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgcggccgcgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactatagtttaaacataacttcgtatagcatacattatacgaagttatattaactcgagtcgacggatccagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggacaggtccggtaagccggagttaaccgcaacatccgtcgagaagtttttgattgagaagtttgattctgtttccgacttaatgcaactttcagagggagaagagtcaagagccttctctttcgacgtaggtggaagaggttacgtactaagagtgaacagttgtgcagacggcttttacaaagataggtatgtataccgtcactttgcatctgctgccttgcctatcccggaggtattagatatcggggagttttctgaatcattgacctactgcatttcaaggagagcccagggcgttacattgcaagacttgccggagaccgaattacctgctgttctgcaacctgtcgctgaggcaatggacgcaatagctgcagcagacttatctcaaacctctgggtttggtcctttcggtcctcaaggtattggtcaatacactacatggcgtgatttcatatgtgccatcgcagacccacacgtttaccattggcagactgtcatggatgatacagtttccgccagtgtagcacaagctttagacgagttaatgctatgggctgaagattgtcccgaagtgagacatttagtgcatgcagatttcggtagcaacaacgttcttacagataatggacgtatcaccgcagttattgattggtctgaggctatgtttggtgattcacaatatgaagtggccaatatcttcttttggaggccatggctggcttgcatggagcaacagacaaggtacttcgaaagaagacaccctgaattggctggtagtccaaggttgagagcctatatgctgagaattggcttagatcagttataccaaagtttagtagatggtaactttgacgatgctgcctgggcacaaggtagatgcgatgcaatagttaggtctggtgctggcacggtaggtagaacgcaaattgccagaagaagtgcagccgtttggacggacggatgtgtagaagttctagccgattctggaaatagacgtccctccacgcgtccaagagctaaagaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgagaattcctcgaggatatcgaactgattcataacttcgtatagcatacattatacgaagttatttaattaagtatacttcttttttttactttgttcagaacaacttctcatttttttctactcataactttagcatcacaaaatacgcaataataacgagtagtaacacttttatagttcatacatgcttcaactacttaataaatgattgtatgataatgttttcaatgtaagagatttcgattatccacaaactttaaaacacagggacaaaattcttgatatgctttcaaccgctgcgttttggatacctattcttgacatgatatgactaccattttgttattgtacgtggggcagttgacgtcttatcatatgtcaaagtcatttgcgaagttcttggcaagttgccaactgacgagatgcagtaaaaagagattgccgtcttgaaactttttgtccttttttttggcgcgccctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 20 - DNA, 7678 bp, Escherichia coli
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgcggccgcggaatagtgacgttgtgatgcggtgagttcggcggttaggggaatggtatatgataaaaaacggaaacgtgcttctttaatttaattgtttaatattgttgcagatatataaaaagggggaaagaaccaaagatgtaattatttctttattgcctcaacctaaagcaagcaataaggtatagagatcaggacgtctcgagagctgatatcaaatttgaagccacgcaagtaactacgtaggtcagagggcacaaggaataacacgtgacatttttcttttttcttttttttttttttttttttttttgttagtcttggcttctgtgccgtagtctgtatacggttttagatgcggtatgtttatcatcgcccagaaatttgcggggtgcaaagaaataaaatccgtgctgaaacccgtgctgaaatccgtgcaccgcatcaaattttctcggaggattctttgcgccggttttcattttcttccacggaataccaagcccattgcatcgcgatgttagtttaggtttaaacataacttcgtatagcatacattatacgaagttatattaactcgagtcgacggatccagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggacaggtccggtaagccggagttaaccgcaacatccgtcgagaagtttttgattgagaagtttgattctgtttccgacttaatgcaactttcagagggagaagagtcaagagccttctctttcgacgtaggtggaagaggttacgtactaagagtgaacagttgtgcagacggcttttacaaagataggtatgtataccgtcactttgcatctgctgccttgcctatcccggaggtattagatatcggggagttttctgaatcattgacctactgcatttcaaggagagcccagggcgttacattgcaagacttgccggagaccgaattacctgctgttctgcaacctgtcgctgaggcaatggacgcaatagctgcagcagacttatctcaaacctctgggtttggtcctttcggtcctcaaggtattggtcaatacactacatggcgtgatttcatatgtgccatcgcagacccacacgtttaccattggcagactgtcatggatgatacagtttccgccagtgtagcacaagctttagacgagttaatgctatgggctgaagattgtcccgaagtgagacatttagtgcatgcagatttcggtagcaacaacgttcttacagataatggacgtatcaccgcagttattgattggtctgaggctatgtttggtgattcacaatatgaagtggccaatatcttcttttggaggccatggctggcttgcatggagcaacagacaaggtacttcgaaagaagacaccctgaattggctggtagtccaaggttgagagcctatatgctgagaattggcttagatcagttataccaaagtttagtagatggtaactttgacgatgctgcctgggcacaaggtagatgcgatgcaatagttaggtctggtgctggcacggtaggtagaacgcaaattgccagaagaagtgcagccgtttggacggacggatgtgtagaagttctagccgattctggaaatagacgtccctccacgcgtccaagagctaaagaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgagaattcctcgaggatatcgaactgattcataacttcgtatagcatacattatacgaagttatttaattaacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgtctgaattcgctactagcggcgttgaaagtggctctcaacaaacttctatccactctactccgatagtgcagaaattagagacggatgaatctcctattcaaaccaaatctgaatacactaacgctgaactcccagcaaagccaatcgccgcatattggactgttatctgtttatgtctaatgattgcatttggtgggtttgtctttggttgggatactggtaccatctctggttttgttaatcaaaccgatttcaaaagaagatttggtcaaatgaaatctgatggtacctattatctttcggacgtccggactggtttgatcgttggtatcttcaatattggttgtgccattggtgggttaaccttaggacgtctgggtgatatgtatggacgtagaattggtttgatgtgcgtcgttctggtatacatcgttggtattgtgattcaaattgcttctagtgacaaatggtaccagtatttcattggtagaattatctctggtatgggtgtcggtggtattgctgtcctatctccaactttgatttccgaaacagcaccaaaacacattagaggtacctgtgtttctttctatcagttaatgatcactctaggtattttcttaggttactgtaccaactatggtactaaagactactccaattcagttcaatggagagtgcctttgggtttgaactttgccttcgctattttcatgatcgctggtatgctaatggttccagaatctccaagattcttagtcgaaaaaggcagatacgaagacgctaaacgttctttggcaaaatctaacaaagtcaccattgaagatccaagtattgttgctgaaatggatacaattatggccaacgttgaaactgaaagattagccggtaacgcttcttggggtgagttattctccaacaaaggtgctattttacctcgtgtgattatgggtattatgattcaatccttacaacaattaactggtaacaattacttcttctattatggtactactattttcaacgccgtcggtatgaaagattctttccaaacttccatcgttttaggtatagtcaacttcgcatccactttcgtggccctatacactgttgataaatttggtcgtcgtaagtgtctattgggcggttctgcttccatggccatttgttttgttatcttctctactgtcggtgtcacaagcttatatccaaatggtaaagatcaaccatcttccaaggctgccggtaacgtcatgattgtctttacctgtttattcattttcttcttcgctattagttgggccccaattgcctacgttattgttgccgaatcttatcctttgcgtgtcaaaaatcgtgctatggctattgctgttggtgccaactggatttggggtttcttgattggtttcttcactcccttcattacaagtgcaattggattttcatacgggtatgtcttcatgggctgtttggtattttcattcttctacgtgtttttctttgtctgtgaaaccaagggcttaacattagaggaagttaatgaaatgtatgttgaaggtgtcaaaccatggaaatctggtagctggatctcaaaagaaaaaagagtttccgaggaataaaagggaaccttttacaacaaatatttgaaaaattacctccattattataccttctctttatgtaattgttagttcgaaaattttttcttcattaatataatcaacttctaaaactttctaaaaacgttctctttttcgagattagtgcttcttcccaatccgtaagaaatgtttcctttcttgacaattggcaccagctggctactcgttgctcgaaaactactctcttttatttttaatttacgaacgattatctttcgaaggaacgaccaaacgagctaaatatgggcgcgatcgctaagtacagacggaaactcacaccgccgcgaagactggtcagtggcaaaaaaaaaataaaaatatagaaaataactattacgtatgttactgtttctggtagttgatatgaagttggagttgtatattgtacgctttaggaacagggaagtgaatattatttactctgctgcacattctggctaggtcgaagccggaacttgagaagacgccgcgctagaactatggaccaagctgttgacaatgttcagatggtgatgcactaccctgtgcggggagtggccacggacgcgagcggaaggtgcggaaggtgcggaaggtgcgggagttgcgggaggttcttcgctaagcgtgagggttgctagctggggcggcggggtttccctaagtgtaaataaggcctcgccgctggcacatgagtgcgccggaggaggcggcggaggcgacgacgctaaaaccgtggccgttggggaaggatgggcggctatatctaccattgacctgatggggactcggttcttaaggaatgggtttgaggtgggtgtggttgcagcaacatctgtagggcgcgccctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacag

SEQ ID NO: 21 - DNA, 7665 bp, Escherichia coli
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgcggccgcggaatagtgacgttgtgatgcggtgagttcggcggttaggggaatggtatatgataaaaaacggaaacgtgcttctttaatttaattgtttaatattgttgcagatatataaaaagggggaaagaaccgaagatgtaattatttttttatcgcctcaacctaaagcaagcaataaggtataaagatcaggacgtctcgagcgctgatatctaaatttgaagccacgcaagtaactacgtaggtcagaggggacaaggaataacacttgacatttttcttttttctttttttttcttttttttttttttgttaatcttggcttctgtaccgtagcctcctgtatacggttttagatgcagtatgtttatcatcgccgagaaatttggggggtgcaaaggaataaaatccgtgctgaaatccgtgcaccgcatcaaactttctcggaggattctttgcaccggttttcattttcttccacggaataccaagcccattgcaatgcgatgttagtttaggtttaaacataacttcgtatagcatacattatacgaagttatattaactcgagtcgacggatccagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggacaggtccggtaagccggagttaaccgcaacatccgtcgagaagtttttgattgagaagtttgattctgtttccgacttaatgcaactttcagagggagaagagtcaagagccttctctttcgacgtaggtggaagaggttacgtactaagagtgaacagttgtgcagacggcttttacaaagataggtatgtataccgtcactttgcatctgctgccttgcctatcccggaggtattagatatcggggagttttctgaatcattgacctactgcatttcaaggagagcccagggcgttacattgcaagacttgccggagaccgaattacctgctgttctgcaacctgtcgctgaggcaatggacgcaatagctgcagcagacttatctcaaacctctgggtttggtcctttcggtcctcaaggtattggtcaatacactacatggcgtgatttcatatgtgccatcgcagacccacacgtttaccattggcagactgtcatggatgatacagtttccgccagtgtagcacaagctttagacgagttaatgctatgggctgaagattgtcccgaagtgagacatttagtgcatgcagatttcggtagcaacaacgttcttacagataatggacgtatcaccgcagttattgattggtctgaggctatgtttggtgattcacaatatgaagtggccaatatcttcttttggaggccatggctggcttgcatggagcaacagacaaggtacttcgaaagaagacaccctgaattggctggtagtccaaggttgagagcctatatgctgagaattggcttagatcagttataccaaagtttagtagatggtaactttgacgatgctgcctgggcacaaggtagatgcgatgcaatagttaggtctggtgctggcacggtaggtagaacgcaaattgccagaagaagtgcagccgtttggacggacggatgtgtagaagttctagccgattctggaaatagacgtccctccacgcgtccaagagctaaagaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgagaattcctcgaggatatcgaactgattcataacttcgtatagcatacattatacgaagttatttaattaacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgtctgaattcgctactagcggcgttgaaagtggctctcaacaaacttctatccactctactccgatagtgcagaaattagagacggatgaatctcctattcaaaccaaatctgaatacactaacgctgaactcccagcaaagccaatcgccgcatattggactgttatctgtttatgtctaatgattgcatttggtgggtttgtctttggttgggatactggtaccatctctggttttgttaatcaaaccgatttcaaaagaagatttggtcaaatgaaatctgatggtacctattatctttcggacgtccggactggtttgatcgttggtatcttcaatattggttgtgccattggtgggttaaccttaggacgtctgggtgatatgtatggacgtagaattggtttgatgtgcgtcgttctggtatacatcgttggtattgtgattcaaattgcttctagtgacaaatggtaccagtatttcattggtagaattatctctggtatgggtgtcggtggtattgctgtcctatctccaactttgatttccgaaacagcaccaaaacacattagaggtacctgtgtttctttctatcagttaatgatcactctaggtattttcttaggttactgtaccaactatggtactaaagactactccaattcagttcaatggagagtgcctttgggtttgaactttgccttcgctattttcatgatcgctggtatgctaatggttccagaatctccaagattcttagtcgaaaaaggcagatacgaagacgctaaacgttctttggcaaaatctaacaaagtcaccattgaagatccaagtattgttgctgaaatggatacaattatggccaacgttgaaactgaaagattagccggtaacgcttcttggggtgagttattctccaacaaaggtgctattttacctcgtgtgattatgggtattatgattcaatccttacaacaattaactggtaacaattacttcttctattatggtactactattttcaacgccgtcggtatgaaagattctttccaaacttccatcgttttaggtatagtcaacttcgcatccactttcgtggccctatacactgttgataaatttggtcgtcgtaagtgtctattgggcggttctgcttccatggccatttgttttgttatcttctctactgtcggtgtcacaagcttatatccaaatggtaaagatcaaccatcttccaaggctgccggtaacgtcatgattgtctttacctgtttattcattttcttcttcgctattagttgggccccaattgcctacgttattgttgccgaatcttatcctttgcgtgtcaaaaatcgtgctatggctattgctgttggtgccaactggatttggggtttcttgattggtttcttcactcccttcattacaagtgcaattggattttcatacgggtatgtcttcatgggctgtttggtattttcattcttctacgtgtttttctttgtctgtgaaaccaagggcttaacattagaggaagttaatgaaatgtatgttgaaggtgtcaaaccatggaaatctggtagctggatctcaaaagaaaaaagagtttccgaggaataaaagggaaccttttacaacaaatatttgaaaaattacctccattattataccttctctttatgtaattgttagttcgaaaattttttcttcattaatataatcaacttctaaaactttctaaaaacgttctctttttcgagattagtgcttcttcccaatccgtaagaaatgtttcctttcttgacaattggcaccagctggctactcgttgctcgaaaactactctcttttatttttaatttacgaacgattatctttcgaaggaacgaccaaacgagctaaatatgggcgcgatcgctaagtacagacggaaactcacaccgccgcgaagactggtcagtggcaaaaaaaaaaaaattaaaaaaataaaaaataactattacgtatgatactgtttctggtagttgatatgaggttggtgttgtatattgtacgttttaggaacagggaagtgaatattatttactctgctgcacattctggctaggtcgaagccggaacctgagaagacgccgcgctagaactatggaccaagctgttgacaatgtttagatggtgatgcactaccctgtgcggggagtggccacgaacgcgagcggaaggtgcgggtgttgcgggaattgcgggaggttcttcgctaagcgtgagggttgctagctggggcggcggggtttccctaagtgtaaatagggcctcgccgctggcacatgagtgcgccggaggaggcggcggaggcgacgacgctaaaaccgtggccgttggggaaggatgggcggctatatctaccattgacctgatggggactcggctcttaaggaatgggttcgaggtgggtgtggttgcagcaacatctgtagggcgcgccctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacag

SEQ ID NO: 22 - protein, 591 aa, Saccharomyces cerevisiae
MLCSVIQRQTREVSNTMSLDSYYLGFDLSTQQLKCLAINQDLKIVHSETVEFEKDLPHYHTKKGVYIHGDTIECPVAMWLGALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFMYELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWIAIIPRLSP

SEQ ID NO: 23 - DNA, 18 bp, Artificial Sequence
  Artificial DNA Primer
gggccctccttactgctc

SEQ ID NO: 24 - DNA, 20 bp, Artificial Sequence
  Artificial DNA Primer
tagacgcagtacaaggacgc

SEQ ID NO: 25 - DNA, 27 bp, Artificial Sequence
  Artificial DNA Primer
tgcaattcaataaatgggatgtgattg

SEQ ID NO: 26 - DNA, 33 bp, Artificial Sequence
  Artificial DNA Primer
gagcgaacgtaagagaggttaatgtcctctaac

SEQ ID NO: 27 - DNA, 22 bp, Artificial Sequence
  Artificial DNA Primer
gatgatcgagccggtagttaac

SEQ ID NO: 28 - DNA, 20 bp, Artificial Sequence
  Artificial DNA Primer
tagacgcagtacaaggacgc

SEQ ID NO: 29 - DNA, 2649 bp, Artificial Sequence
  Artificial DNA Sequence
gggccctccttactgctctccttccgtgtaacgcgttatgaaactctaatagctacctatattccaccataatatcaatcatgcggttgctggtgtatttaccaataatgtttaatgtatatatattaggggccgtatacttacatatagtagatgtcaagcgtaggcgcttcccctgccggctgtgacggcgccataaccaaggtatctatagaccgccaatcagcaaactacctccgtacattcatgttgcacccacacatgtacacacccagaccgcaacaaattacccataaggttgtttgtgacggcgtcgtacaagagaacgtgggaactttttaggctcaccaaaaaagaaaggaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctattttttttttttccatttttctttctctctttctaatatataaattctcttgcattttctatttttctctctatctattctacttgtttattcccttcaaggtttttttttaaggagtacttgtttttagaatatacggtcaacgaactataattaaatgtctgaattcgctactagcggcgttgaaagtggctctcaacaaacttctatccactctactccgatagtgcagaaattagagacggatgaatctcctattcaaaccaaatctgaatacactaacgctgaactcccagcaaagccaatcgccgcatattggactgttatctgtttatgtctaatgattgcatttggtgggtttgtctttggttgggatactggtaccatctctggttttgttaatcaaaccgatttcaaaagaagatttggtcaaatgaaatctgatggtacctattatctttcggacgtccggactggtttgatcgttggtatcttcaatattggttgtgccattggtgggttaaccttaggacgtctgggtgatatgtatggacgtagaattggtttgatgtgcgtcgttctggtatacatcgttggtattgtgattcaaattgcttctagtgacaaatggtaccagtatttcattggtagaattatctctggtatgggtgtcggtggtattgctgtcctatctccaactttgatttccgaaacagcaccaaaacacattagaggtacctgtgtttctttctatcagttaatgatcactctaggtattttcttaggttactgtaccaactatggtactaaagactactccaattcagttcaatggagagtgcctttgggtttgaactttgccttcgctattttcatgatcgctggtatgctaatggttccagaatctccaagattcttagtcgaaaaaggcagatacgaagacgctaaacgttctttggcaaaatctaacaaagtcaccattgaagatccaagtattgttgctgaaatggatacaattatggccaacgttgaaactgaaagattagccggtaacgcttcttggggtgagttattctccaacaaaggtgctattttacctcgtgtgattatgggtattatgattcaatccttacaacaattaactggtaacaattacttcttctattatggtactactattttcaacgccgtcggtatgaaagattctttccaaacttccatcgttttaggtatagtcaacttcgcatccactttcgtggccctatacactgttgataaatttggtcgtcgtaagtgtctattgggcggttctgcttccatggccatttgttttgttatcttctctactgtcggtgtcacaagcttatatccaaatggtaaagatcaaccatcttccaaggctgccggtaacgtcatgattgtctttacctgtttattcattttcttcttcgctattagttgggccccaattgcctacgttattgttgccgaatcttatcctttgcgtgtcaaaaatcgtgctatggctattgctgttggtgccaactggatttggggtttcttgattggtttcttcactcccttcattacaagtgcaattggattttcatacgggtatgtcttcatgggctgtttggtattttcattcttctacgtgtttttctttgtctgtgaaaccaagggcttaacattagaggaagttaatgaaatgtatgttgaaggtgtcaaaccatggaaatctggtagctggatctcaaaagaaaaaagagtttccgaggaataaaagggaaccttttacaacaaatatttgaaaaattacctccattattataccttctctttatgtaattgttagttcgaaaattttttcttcattaatataatcaacttctaaaactttctaaaaacgttctctttttcgagattagtgcttcttcccaatccgtaagaaatgtttcctttcttgacaattggcaccagctggctactcgttgctcgaaaactactctcttttatttttaatttacgaacgattatctttcgaaggaacgaccaaacgagctaaatatgggcatcggcgactctctcgaaatttttcttaacgcgtccttgtactgcgtcta

SEQ ID NO: 30 - DNA, 26 bp, Artificial Sequence
  Artificial DNA Primer
agcacaatccaaggaaaaatctggcc

SEQ ID NO: 31 - DNA, 34 bp, Artificial Sequence
  Artificial DNA Primer
gccattagtagtgtactcaaacgaattattgttg

SEQ ID NO: 32 - DNA, 40 bp, Artificial Sequence
  Artificial DNA Primer
tcagtactgacaataaaaagattcttgttttcaagaactt

SEQ ID NO: 33 - DNA, 21 bp, Artificial Sequence
  Artificial DNA Primer
tagcgtgttacgcacccaaac

SEQ ID NO: 34 - DNA, 22 bp, Artificial Sequence
  Artificial DNA Primer
acagaagacgggagacactagc

SEQ ID NO: 35 - DNA, 38 bp, Artificial Sequence
  Artificial DNA Primer
tttgtttgtttatgtgtgtttattcgaaactaagttct

SEQ ID NO: 36 - DNA, 35 bp, Artificial Sequence
  Artificial DNA Primer
agttgattgtatgcttggtatagcttgaaatattg

SEQ ID NO: 37 - DNA, 49 bp, Artificial Sequence
  Artificial DNA Primer
tgttttatatttgttgtaaaaagtagataattacttccttgatgatctg

SEQ ID NO: 38 - DNA, 48 bp, Artificial Sequence
  Artificial DNA Primer
tttgttttttgttttcttctaattgattttttctttctatttcctttg

SEQ ID NO: 39 - DNA, 18 bp, Artificial Sequence
  Artificial DNA Primer
ggggtcgcaacttttccc

SEQ ID NO: 40 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 41 - protein, 334 aa, Candida glabrata
MSEPVQKKQKTNSSLDQLKASGTVVVADTGDFESIAKFQPQDSTTNPSLILAAAKQPAYAKLIDVAVEYGKKHGKTVEEQTEAAVDRLLVEFGKEILKIVPGRVSTEVDARLSFDKEATIAKALQIIKLYEEQGISKSRVLIKIASTWEGIQAARELESKHGIHCNLTLLFNFAQAVACAEANITLISPFVGRIMDYYKAKTGETYTGETDPGVKSVRAIYNYYKKYGYKTIVMGASFRNIDEIKALAGVDYLTISPNLLDQLLNSNDPVPKILDPATAKDEAGEKVTFVDNESAFRFALNDDAMATDKLSDGIRKFSADIITLFDMIEKKVKA

SEQ ID NO: 42 - protein, 335 aa, Saccharomyces dairenensi
MSEPVQKKQKVTSSSLEQLKASGTVVVADTGDFASIAKFTPQDATTNPSLILAAAKQSAYAKLIDVAVEYGKKHGKTTEEKTEIAVDRLLVEFGKEILAIVPGRVSTEVDARLSFDKEATIAKALEIIKLYKDIGISKERVLIKIASTWEGIQAARELESKHGIHCNLTLLFSFSQAVACAEANVTLISPFVGRIMDWHKAKTGETYTGRNDPGVLSVKKIYNYYKKYDYKTIVMGASFRNVDEIKNLAGVDFLTISPSLLDELLNSQEPVPRVLDVASAKKENIPKVSFIDDESTFRFELNEDAMATEKLAEGIRKFSADIVTLFDLIEKKVAA

SEQ ID NO: 43 - protein, 363 aa, Spathaspora girioi
MVANPSLVLKKIDEIVFETPEAPEISEPTDVIVQVKKTGICGSDIHFYAHGKIGNYILTKPMVLGHESAGVVTQIGSGVKNLRVGDNVAIEPGVPSRYSNAYKSGRYNLCPFMRFAATPTSEKDEPNPPGTLCKYFKSPEDFLVKLPDHVSLELGAMVEPLSVGVHACKLGSVKFGDTVAVFGAGPVGILTAATAKTFGASKVIIIDVFDNKLQMAKDIGVVTHTFNSKSDGDYNDLIKHFGEQPSVVLECTGADPCVGMGVNICAPGGRFIQVGNAAAPVKFPITVFAMKELTLFGSFRYGYGDYQDAVNIFDANYKNGKENAPIDFERLITHRFKFDDAIKAYDLVRSGCGSVKCLIDGPE

SEQ ID NO: 44 - protein, 362 aa, Candida tenuis
MVSNPSLVLNEIKNLEFQTHQAPEATEDFDVLVEVKKTGICGSDVHYYLHGEIGSFKLNKPMVMGHESSGIVSKIGPKVTSLKVGDRVAIEPGLPSRFSDEYKSGHYNLCPHMCFAATPAPEGTPNPPGTLCKYYKCPEDFLVKLPETVSLELGALVEPLTVGVHASKLANVKFGDVVVIFGAGPVGLLAASVATVFGASAVCVVDIFDNKLQMAKDIGAATHVFNSKTEGGYTQLVKKLGKSPTVVLECTGAEVCIQMGVLALATGGRFVQVGNAQGYVKFPITEFATKELQLFGSFRYGYNDYKTAVALLEKNYRNGKENVIVDFEKLITHRYSFKDAIKAYEEVAAGNGAVKCMIDGPE

SEQ ID NO: 45 - protein, 498 aa, Pseudomonas fluorescens
MTQPALFLGLDCGTQGTKALILDSRSGTVLGRGAAPHSLISGANGCREQDPAQWLQACTSATRQALQAAGVDGRQVLGVGVCGQQHGLVLLDDQGQVLRPAKLWCDTQSSAENQRLLDWLGGTQGSLERLGLAIAPGYTVSKLLWTREQHPELFQRIAHILLPHDYLNFWLTGRCCSEYGDASGSGYFDVRRRDWDRALLAHIDPSGRLERALPPLLEAHQPVGRILAPVARQLGISADAWVASGGGDNMLGAIGTGNIQPGIITMSLGSSGTVYAYAERPLISPEPSVATFCSSSGGWLPLICTMNLTNATTLVRELLGLDLAAFNQRLEQAPIGAEGLCLLPFFNGERVPALPQAQGSLHGMTLDNLTPANLCRAVVEGTTFGLRYGLDLLRAAGLHSQSIRLIGGGAKSLLWRQMVANIMHAPVICPREAEAAALGAAIQAAWCHGRQDDPDLDLQQLCERCVRLDPGSETQPRAEHVAAYEPVYQRYRQQLASL

SEQ ID NO: 46 - protein, 623 aa, Scheffersomyces stipitis
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLYTDGKKQTFESLTARPNRCYYVGGASNNGSIIRKMGSILAPVNGNYKVDIPNACALGGAYKASWSYECEAKKEWIGYDQYINRLFEVSDEMNSFEVKDKWLEYANGVGMLAKMESELKH

SEQ ID NO: 47 - protein, 319 aa, Aspergillus niger
MASPTVKLNSGYDMPLVGFGLWKVNNDTCADQIYHAIKEGYRLFDGACDYGNEVEAGQGIARAIKDGLVKREELFIVSKLWNSFHDGDRVEPICRKQLADWGIDYFDLYIVHFPISLKYVDPAVRYPPGWKSEKDELEFGNATIQETWTAMESLVDKKLARSIGISNFSAQLVMDLLRYARIRPATLQIEHHPYLTQTRLVEYAQKEGLTVTAYSSFGPLSFLELSVQNAVDSPPLFEHQLVKSIAEKHGRTPAQVLLRWATQRGIAVIPKSNNPQRLKQNLDVTGWNLEEEEIKAISGLDRGLRFNDPLGYGLYAPIF

SEQ ID NO: 48 - protein, 319 aa, Aspergillus oryzae
MAPPTVKLNSGFDMPLVGFGLWKVNNETCADQVYEAIKAGYRLFDGACDYGNEVECGQGVARAIKEGIVKREDLFIVSKLWNSFHEGDRVEPVCRKQLADWGVEYFDLYIVHFPVALKYVDPAVRYPPGWNSESGKIEFSNASIQETWTAMESLVDKKLARSIGVSNFSAQLLMDLLRYARVRPATLQIEHHPYLTQPRLVEYAQKEGIAVTAYSSFGPLSFLELEVKNAVNTTPLFEHNTIKSLAEKYGKTPAQVLLRWATQRGIAVIPKSNNPTRLALNLEVTGWDLEKTELEAISSLDQGLRFNDPLGYGMYVPIF

SEQ ID NO: 49 - protein, 569 aa, Saccharomyces cerevisiae
MSFQIETVPTKPYEDQKPGTSGLRKKTKVFKDEPNYTENFIQSIMEAIPEGSKGATLVVGGDGRYYNDVILHKIAAIGAANGIKKLVIGQHGLLSTPAASHIMRTYEEKCTGGIILTASHNPGGPENDMGIKYNLSNGGPAPESVTNAIWEISKKLTSYKIIKDFPELDLGTIGKNKKYGPLLVDIIDITKDYVNFLKEIFDFDLIKKFIDNQRSTKNWKLLFDSMNGVTGPYGKAIFVDEFGLPADEVLQNWHPSPDFGGMHPDPNLTYASSLVKRVDREKIEFGAASDGDGDRNMIYGYGPSFVSPGDSVAIIAEYAAEIPYFAKQGIYGLARSFPTSGAIDRVAKAHGLNCYEVPTGWKFFCALFDAKKLSICGEESFGTGSNHVREKDGVWAIMAWLNILAIYNKHHPENEASIKTIQNEFWAKYGRTFFTRYDFEKVETEKANKIVDQLRAYVTKSGVVNSAFPADESLKVTDCGDFSYTDLDGSVSDHQGLYVKLSNGARFVLRLSGTGSSGATIRLYIEKYCDDKSQYQKTAEEYLKPIINSVIKFLNFKQVLGTEEPTVRT

SEQ ID NO: 50 - DNA, 633 bp, Saccharomyces cerevisiae
agctacctatattccaccataatatcaatcatgcggttgctggtgtatttaccaataatgtttaatgtatatatattaggggccgtatacttacatatagtagatgtcaagcgtaggcgcttcccctgccggctgtgacggcgccataaccaaggtatctatagaccgccaatcagcaaactacctccgtacattcatgttgcacccacacatgtacacacccagaccgcaacaaattacccataaggttgtttgtgacggcgtcgtacaagagaacgtgggaactttttaggctcaccaaaaaagaaaggaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctattttttttttttccatttttctttctctctttctaatatataaattctcttgcattttctatttttctctctatctattctacttgtttattcccttcaaggtttttttttaaggagtacttgtttttagaatatacggtcaacgaactataattaa

SEQ ID NO: 51 - DNA, 290 bp, Saccharomyces cerevisiae
aagggaaccttttacaacaaatatttgaaaaattacctccattattataccttctctttatgtaattgttagttcgaaaattttttcttcattaatataatcaacttctaaaactttctaaaaacgttctctttttcgagattagtgcttcttcccaatccgtaagaaatgtttcctttcttgacaattggcaccagctggctactcgttgctcgaaaactactctcttttatttttaatttacgaacgattatctttcgaaggaacgaccaaacgagctaaatatgggc